(19) United States
(12) Patent Application Publication
(10) Pub. No.: US 2012/0195857 A1
(43) Pub. Date: Aug. 2, 2012

(54) Hepatitis C Virus Inhibitors

(75) Inventors: Makonen Belema, North Haven, CT (US); Pothukanuri Srinivasu, Bangalore (IN); John A. Bender, Middletown, CT (US); Omar D. Lopez, Wallingford, CT (US); Qi Chen, Stamford, CT (US); Richard A. Rampulla, Flemington, NJ (US); Samayamunthula Venkata Satya Arun Kumar Gupta, Bangalore (IN); Nicholas A. Meanwell, East Hampton, CT (US)

(73) Assignee: Bristol-Myers Squibb Company

(21) Appl. No.: 13/198,529

(22) Filed: Aug. 4, 2011

Related U.S. Application Data
(60) Provisional application No. 61/373,070, filed on Aug. 12, 2010.

(51) Int. Cl. A61K 31/4178; C07D 413/14; A61K 31/422; C07D 417/14; A61K 31/433; A61P 31/14; A61K 31/5025; A61K 31/7056; A61K 38/21; A61K 38/48; A61K 38/44; A61K 38/46; C07D 403/14; C07D 487/04

(52) U.S. Cl. 424/85.7; 548/313.1; 514/397; 548/312.1; 548/235; 514/374; 548/134; 514/362; 544/349; 514/249; 514/43; 424/85.4; 424/94.67; 424/94.64; 424/94.5; 424/94.6; 514/4.3

(57) ABSTRACT

The present disclosure relates to compounds, compositions and methods for the treatment of hepatitis C virus (HCV) infection. Also disclosed are pharmaceutical compositions containing such compounds and methods for using these compounds in the treatment of HCV infection.

### CROSS-REFERENCE TO RELATED APPLICATIONS

This application claims the benefit of U.S. Provisional Application Ser. No. 61/373,070 filed Aug. 12, 2010.

The present disclosure is generally directed to antiviral compounds, and more specifically directed to compounds which can inhibit the function of the NS5A protein encoded by Hepatitis C virus (HCV), compositions comprising such compounds, and methods for inhibiting the function of the NS5A protein.

HCV is a major human pathogen, infecting an estimated 170 million persons worldwide—roughly five times the number infected by human immunodeficiency virus type 1. A substantial fraction of these HCV infected individuals develop serious progressive liver disease, including cirrhosis and hepatocellular carcinoma.

The current standard of care for HCV, which employs a combination of pegylated-interferon and ribavirin, has a non-optimal success rate in achieving sustained viral response and causes numerous side effects. Thus, there is a clear and long-felt need to develop effective therapies to address this undermet medical need.

HCV is a positive-stranded RNA virus. Based on a comparison of the deduced amino acid sequence and the extensive similarity in the 5′ untranslated region, HCV has been classified as a separate genus in the Flaviviridae family. All members of the Flaviviridae family have enveloped virions that contain a positive stranded RNA genome encoding all known virus-specific proteins via translation of a single, uninterrupted, open reading frame.

Considerable heterogeneity is found within the nucleotide and encoded amino acid sequence throughout the HCV genome due to the high error rate of the encoded RNA dependent RNA polymerase which lacks a proof-reading capability. At least six major genotypes have been characterized, and more than 50 subtypes have been described with distribution worldwide. The clinical significance of the genetic heterogeneity of HCV has demonstrated a propensity for mutations to arise during monotherapy treatment, thus additional treatment options for use are desired. The possible modulator effect of genotypes on pathogenesis and therapy remains elusive.

The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature non-structural proteins (NS2, NS3, NS4A, NS4B, NS5A, and NS5B) is effected by two viral proteases. The first one is believed to be a metalloprotease and cleaves at the NS2-NS3 junction; the second one is a serine protease contained within the N-terminal region of NS3 (also referred to herein as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions by both acting as a cofactor for the NS3 protease and assisting in the membrane localization of NS3 and other viral replicase components. The formation of a NS3-NS4A complex is necessary for proper protease activity resulting in increased proteolytic efficiency of the cleavage events. The NS3 protein also exhibits nucleoside triphosphatase and RNA helicase activities. NS5B (also referred to herein as HCV polymerase) is a RNA-dependent RNA polymerase that is involved in the replication of HCV with other HCV proteins, including NS5A, in a replicase complex.

Compounds useful for treating HCV-infected patients are desired which selectively inhibit HCV viral replication. In particular, compounds which are effective to inhibit the function of the NS5A protein are desired. The HCV NS5A protein is described, for example, in the following references: S. L. Tan, et al., Virology, 284:1-12 (2001); K.-J. Park, et al., J. Biol. Chem., 30711-30718 (2003); T. L. Tellinghuisen, et al., Nature, 435, 374 (2005); R. A. Love, et al., J. Virol, 83, 4395 (2009); N. Appel, et al., J. Biol. Chem., 281, 9833 (2006); L. Huang, J. Biol. Chem., 280, 36417 (2005); C. Rice, et al., WO2006093867.

In a first aspect the present disclosure provides a compound of Formula (I)

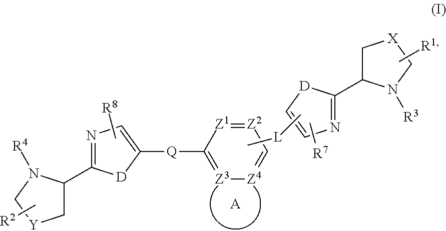

or a pharmaceutically acceptable salt thereof, wherein

each D is independently selected from O and NH;

L is a bond or phenyl;

Q is selected from phenyl, a six-membered heteroaromatic ring containing one, two, or three nitrogen atoms, and

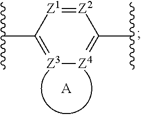

X is selected from O, S, CH₂, CH₂CH₂, (NR¹)CH₂, and OCH₂,

Y is selected from O, S, CH₂, CH₂CH₂, (NR²)CH₂, and OCH₂;

Z¹ and Z² are each independently selected from CH and N;

Z³ and Z⁴ are each independently selected from C and N;

provided that no more than two of Z¹, Z², Z³, and Z⁴ are N;

A is a four- to six-membered ring optionally containing one or two additional double bonds and optionally containing one, two, or three heteroatoms independently selected from nitrogen, oxygen, and sulfur, wherein said ring is optionally substituted with an alkyl group;

R¹ and R² are independently selected from hydrogen, alkyl, halo, and hydroxy; wherein the alkyl can optionally form a fused three- to six-membered ring or a bridged four- or five-membered ring with an another carbon atom on the ring; or can optionally form a spirocyclic three- to six-membered ring with the carbon to which it is attached;

provided that when X is (NR¹)CH₂, R¹ is hydrogen or alkyl; and
provided that when Y is (NR²)CH₂, R² is hydrogen or alkyl;

R³ is selected from hydrogen and —C(O)R⁵;

R⁴ is selected from hydrogen and —C(O)R⁶;

R⁵ and R⁶ are independently selected from alkoxy, alkyl, arylalkoxy, arylalkyl, cycloalkyl, cycloalkyloxy, heterocyclyl, heterocyclylalkyl, (NR^{c}R^{d})alkenyl, and (NR^{c}R^{d})alkyl;

R⁷ and R⁸ are independently selected from hydrogen, alkyl, cyano, and halo;

R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro;

R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR^{x}R^{y})alkyl, and (NR^{s}R^{y})carbonyl; and

R^{x} and R^{y} are independently selected from hydrogen and alkyl.

In a first embodiment of the first aspect the present disclosure provides a compound of formula (I), or a pharmaceutically acceptable salt thereof, wherein Q is phenyl.

In a second embodiment of the first aspect the present disclosure provides a compound of formula (I), or a pharmaceutically acceptable salt thereof, wherein X and Y are each CH₂.

In a third embodiment of the first aspect the present disclosure provides a compound of formula (I), or a pharmaceutically acceptable salt thereof, wherein R⁷ and R⁸ are each hydrogen.

In a second aspect the present disclosure provides a compound of formula (II)

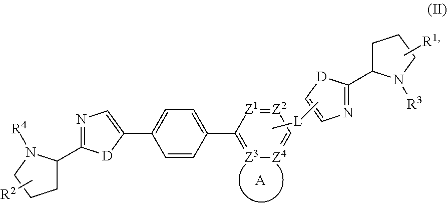

or a pharmaceutically acceptable salt thereof, wherein

each D is independently selected from O and NH;

L is a bond or phenyl;

Z¹ and Z² are each independently selected from CH and N;

Z³ and Z⁴ are each independently selected from C and N;

provided that no more than two of Z¹, Z², Z³, and Z⁴ are N;

A is a four- to six-membered ring optionally containing one or two additional double bonds and optionally containing one, two, or three heteroatoms independently selected from nitrogen, oxygen, and sulfur;

R¹ and R² are independently selected from hydrogen, alkyl, halo, and hydroxy; wherein the alkyl can optionally form a fused three- to six-membered ring or a bridged four- or five-membered ring with an another carbon atom on the ring; or can optionally form a spirocyclic three- to six-membered ring with the carbon to which it is attached;

R³ is selected from hydrogen and —C(O)R⁵;

R⁴ is selected from hydrogen and —C(O)R⁶;

R⁵ and R⁶ are independently selected from alkoxy, alkyl, arylalkoxy, arylalkyl, cycloalkyl, cycloalkyloxy, heterocyclyl, heterocyclylalkyl, (NR^{c}R^{d})alkenyl, and (NR^{c}R^{d})alkyl;

R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro;

R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR^{x}R^{y})alkyl, and (NR^{x}R^{y})carbonyl; and

R^{x} and R^{y} are independently selected from hydrogen and alkyl.

In a third aspect the present disclosure provides a compound of formula (III)

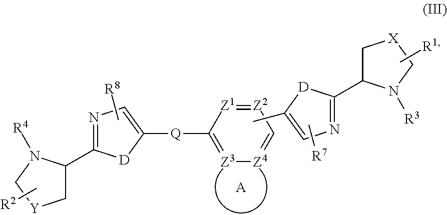

or a pharmaceutically acceptable salt thereof, wherein

each D is independently selected from O and NH;

Q is selected from phenyl, a six-membered heteroaromatic ring containing one, two, or three nitrogen atoms, and

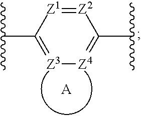

X is selected from O, S, CH₂, CH₂CH₂, (NR¹)CH₂, and OCH₂,

Y is selected from O, S, CH₂, CH₂CH₂, (NR²)CH₂, and OCH₂;

Z¹ and Z² are each independently selected from CH and N;

Z³ and Z⁴ are each independently selected from C and N;

provided that no more than two of Z¹, Z², Z³, and Z⁴ are N;

A is a four- to six-membered ring optionally containing one or two additional double bonds and optionally containing one, two, or three heteroatoms independently selected from nitrogen, oxygen, and sulfur;

R¹ and R² are independently selected from hydrogen, alkyl, halo, and hydroxy; wherein the alkyl can optionally form a fused three- to six-membered ring or a bridged four- or five-membered ring with an another carbon atom on the ring; or can optionally form a spirocyclic three- to six-membered ring with the carbon to which it is attached;

provided that when X is (NR¹)CH₂, R¹ is hydrogen or alkyl; and

provided that when Y is (NR²)CH₂, R² is hydrogen or alkyl;

R³ is selected from hydrogen and —C(O)R⁵;

R⁴ is selected from hydrogen and —C(O)R⁶;

R⁵ and R⁶ are independently selected from alkoxy, alkyl, arylalkoxy, arylalkyl, cycloalkyl, cycloalkyloxy, heterocyclyl, heterocyclylalkyl, (NR^{c}R^{d})alkenyl, and (NR^{c}R^{d})alkyl;

R⁷ and R⁸ are independently selected from hydrogen, alkyl, cyano, and halo;

R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro;

R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR′R^{y})alkyl, and (NR^{x}R^{y})carbonyl; and

R^{x} and R^{y} are independently selected from hydrogen and alkyl.

In a fourth aspect the present disclosure provides a composition comprising a compound of Formula (I), or a pharmaceutically acceptable salt thereof, and a pharmaceutically acceptable carrier. In a first embodiment of the fourth aspect the composition further comprises one or two additional compounds having anti-HCV activity. In a second embodiment of the fourth aspect at least one of the additional compounds is an interferon or a ribavirin. In a third embodiment the interferon is selected from interferon alpha 2B, pegylated interferon alpha, consensus interferon, interferon alpha 2A, and lymphoblastiod interferon tau.

In a fourth embodiment of the fourth aspect the present disclosure provides a composition comprising a compound of Formula (I), or a pharmaceutically acceptable salt thereof, a pharmaceutically acceptable carrier, and one or two additional compounds having anti-HCV activity, wherein at least one of the additional compounds is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

In a fifth embodiment of the fourth aspect the present disclosure provides a composition comprising a compound of Formula (I), or a pharmaceutically acceptable salt thereof, a pharmaceutically acceptable carrier, and one or two additional compounds having anti-HCV activity, wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.

In a fifth aspect the present disclosure provides a method of treating an HCV infection in a patient, comprising administering to the patient a therapeutically effective amount of a compound of Formula (I), or a pharmaceutically acceptable salt thereof. In a first embodiment of the fifth aspect the method further comprises administering one or two additional compounds having anti-HCV activity prior to, after or simultaneously with the compound of Formula (I), or a pharmaceutically acceptable salt thereof. In a second embodiment of the fifth aspect at least one of the additional compounds is an interferon or a ribavirin. In a third embodiment of the fifth aspect the interferon is selected from interferon alpha 2B, pegylated interferon alpha, consensus interferon, interferon alpha 2A, and lymphoblastiod interferon tau.

In a fourth embodiment of the fifth aspect the present disclosure provides a method of treating an HCV infection in a patient, comprising administering to the patient a therapeutically effective amount of a compound of Formula (I), or a pharmaceutically acceptable salt thereof, and one or two additional compounds having anti-HCV activity prior to, after or simultaneously with the compound of Formula (I), or a pharmaceutically acceptable salt thereof, wherein at least one of the additional compounds is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

In a fifth embodiment of the fifth aspect the present disclosure provides a method of treating an HCV infection in a patient, comprising administering to the patient a therapeutically effective amount of a compound of Formula (I), or a pharmaceutically acceptable salt thereof, and one or two additional compounds having anti-HCV activity prior to, after or simultaneously with the compound of Formula (I), or a pharmaceutically acceptable salt thereof, wherein at least one of the additional compounds is effective to inhibit the function of a target selected from HCV metalloprotease, HCV serine protease, HCV polymerase, HCV helicase, HCV NS4B portein, HCV entry, HCV assembly, HCV egress, HCV NS5A protein, and IMPDH for the treatment of an HCV infection.

Other embodiments of the present disclosure may comprise suitable combinations of two or more of embodiments and/or aspects disclosed herein.

Yet other embodiments and aspects of the disclosure will be apparent according to the description provided below.

The compounds of the present disclosure also exist as tautomers; therefore the present disclosure also encompasses all tautomeric forms.

The description of the present disclosure herein should be construed in congruity with the laws and principals of chemical bonding.

It should be understood that the compounds encompassed by the present disclosure are those that are suitably stable for use as pharmaceutical agent.

It is intended that the definition of any substituent or variable (e.g., R^{c} and R^{d}) at a particular location in a molecule be independent of its definitions elsewhere in that molecule.

All patents, patent applications, and literature references cited in the specification are herein incorporated by reference in their entirety. In the case of inconsistencies, the present disclosure, including definitions, will prevail.

As used in the present specification, the following terms have the meanings indicated:

As used herein, the singular forms “a”, “an”, and “the” include plural reference unless the context clearly dictates otherwise.

Unless stated otherwise, all aryl, cycloalkyl, and heterocyclyl groups of the present disclosure may be substituted as described in each of their respective definitions. For example, the aryl part of an arylalkyl group may be substituted as described in the definition of the term ‘aryl’.

The term “alkoxy,” as used herein, refers to an alkyl group attached to the parent molecular moiety through an oxygen atom.

The term “alkyl,” as used herein, refers to a group derived from a straight or branched chain saturated hydrocarbon containing from one to six carbon atoms. In the compounds of the present disclosure, when X and/or Y is CH₂ and R¹ and or R² is alkyl, respectively, the alkyl can optionally form a fused three- to six-membered ring with an adjacent carbon atom to provide one of the structures shown below:

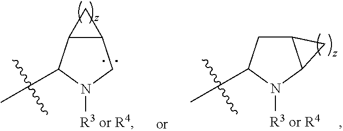

where z is 1, 2, 3, or 4; or, alternatively, the alkyl group can form a four- or five-membered bridged ring to provide one of the structures shown below:

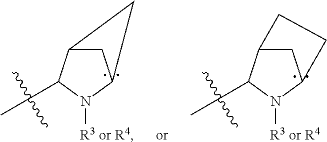

or, alternatively, the alkyl group can form a spirocyclic three- to six-membered ring with the carbon atom to which it is attached to provide one of the structures shown below:

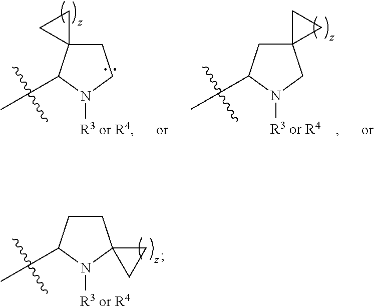

wherein z is 1, 2, 3, or 4.

The term “C₂ alkynyl,” as used herein, refers to -≡-.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl. The aryl groups of the present disclosure are optionally substituted with one, two, three, four, or five substituents independently selected from alkoxy, alkoxyalkyl, alkoxycarbonyl, alkyl, alkylcarbonyl, a second aryl group, arylalkoxy, arylalkyl, arylcarbonyl, cyano, halo, haloalkoxy, haloalkyl, heterocyclyl, heterocyclylalkyl, heterocyclylcarbonyl, hydroxy, hydroxyalkyl, nitro, —NR^{x}R^{y}, (NR^{x}R^{y})alkyl, oxo, and —P(O)OR₂, wherein each R is independently selected from hydrogen and alkyl; and wherein the alkyl part of the arylalkyl and the heterocyclylalkyl are unsubstituted and wherein the second aryl group, the aryl part of the arylalkyl, the aryl part of the arylcarbonyl, the heterocyclyl, and the heterocyclyl part of the heterocyclylalkyl and the heterocyclylcarbonyl are further optionally substituted with one, two, or three substituents independently selected from alkoxy, alkyl, cyano, halo, haloalkoxy, haloalkyl, and nitro.

The term “arylalkyl,” as used herein, refers to an alkyl group substituted with one, two, or three aryl groups. The alkyl part of the arylalkyl is further optionally substituted with one or two additional groups independently selected from alkoxy, alkylcarbonyloxy, halo, haloalkoxy, haloalkyl, heterocyclyl, hydroxy, and —NR^{c}R^{d}, wherein the heterocyclyl is further optionally substituted with one or two substituents independently selected from alkoxy, alkyl, unsubstituted aryl, unsubstituted arylalkoxy, unsubstituted arylalkoxycarbonyl, halo, haloalkoxy, haloalkyl, hydroxy, —NR^{x}R^{y}, and oxo.

The term “cycloalkyl,” as used herein, refers to a saturated monocyclic or bicyclic hydrocarbon ring system having three to fourteen carbon atoms and zero heteroatoms. Representative examples of cycloalkyl groups include, but are not limited to, cyclopropyl, cyclobutyl, cyclopentyl, cyclohexyl, bicyclo[3.1.1]heptyl, and adamantyl. The cycloalkyl groups of the present disclosure are optionally substituted with one, two, three, four, or five substituents independently selected from alkoxy, alkyl, aryl, cyano, halo, haloalkoxy, haloalkyl, heterocyclyl, hydroxy, hydroxyalkyl, nitro, and —NR^{x}R^{y}, wherein the aryl and the heterocyclyl are further optionally substituted with one, two, or three substituents independently selected from alkoxy, alkyl, cyano, halo, haloalkoxy, haloalkyl, hydroxy, nitro, and oxo.

The term “heterocyclyl,” as used herein, refers to a four-, five-, six-, or seven-membered ring containing one, two, three, or four heteroatoms independently selected from nitrogen, oxygen, and sulfur. The four-membered ring has zero double bonds, the five-membered ring has zero to two double bonds, and the six- and seven-membered rings have zero to three double bonds. The term “heterocyclyl” also includes bicyclic groups in which the heterocyclyl ring is fused to another monocyclic heterocyclyl group or a three- to seven-membered aromatic or non-aromatic carbocyclic ring; bicyclic groups in which the heterocyclyl ring is substituted with a three- to seven-membered spirocyclic ring; as well as bridged bicyclic groups such as 3-oxabicyclo[3.2.1]octyl, 7-azabicyclo[2.2.1]hept-7-yl, 2-azabicyclo[2.2.2]oct-2-yl, and 2-azabicyclo[2.2.2]oct-3-yl. The heterocyclyl groups of the present disclosure can be attached to the parent molecular moiety through any carbon atom or nitrogen atom in the group. Examples of heterocyclyl groups include, but are not limited to, benzothienyl, furyl, imidazolyl, indolinyl, indolyl, isoquinolinyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, quinolinyl, tetrahydropyranyl, thiazolyl, thienyl, and thiomorpholinyl. The heterocyclyl groups of the present disclosure are optionally substituted with one, two, three, four, or five substituents independently selected from alkenyl, alkoxy, alkoxyalkyl, alkoxycarbonyl, alkyl, alkylcarbonyl, aryl, arylalkyl, arylcarbonyl, cyano, halo, haloalkoxy, haloalkyl, a second heterocyclyl group, heterocyclylalkyl, heterocyclylcarbonyl, hydroxy, hydroxyalkyl, nitro, —NR^{x}R^{y}, (NR^{x}R^{y})alkyl, and oxo, wherein the alkyl part of the arylalkyl and the heterocyclylalkyl are unsubstituted and wherein the aryl, the aryl part of the arylalkyl, the aryl part of the arylcarbonyl, the second heterocyclyl group, and the heterocyclyl part of the heterocyclylalkyl and the heterocyclylcarbonyl are further optionally substituted with one, two, or three substituents independently selected from alkoxy, alkyl, cyano, halo, haloalkoxy, haloalkyl, and nitro.

The term “heterocyclylalkyl,” as used herein, refers to an alkyl group substituted with one, two, or three heterocyclyl groups. The alkyl part of the heterocyclylalkyl is further optionally substituted with one or two additional groups independently selected from alkoxy, alkylcarbonyloxy, aryl, halo, haloalkoxy, haloalkyl, hydroxy, and —NR^{c}R^{d}, wherein the aryl is further optionally substituted with one or two substituents independently selected from alkoxy, alkyl, unsubstituted aryl, unsubstituted arylalkoxy, unsubstituted arylalkoxycarbonyl, halo, haloalkoxy, haloalkyl, hydroxy, and —NR^{x}R^{y}.

The term “—NR^{c}R^{d},” as used herein, refers to two groups, R^{c} and R^{d}, which are attached to the parent molecular moiety through a nitrogen atom. R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro.

The term “(NR^{c}R^{d})alkenyl,” as used herein, refers to

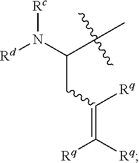

wherein R^{c} and R^{d} are as defined herein and each R^{q} is independently hydrogen or C₁₋₃ alkyl.

The term “(NR^{c}R^{d})alkyl,” as used herein, refers to an alkyl group substituted with one or two —NR^{c}R^{d} groups. The alkyl part of the (NR^{c}R^{d})alkyl is further optionally substituted with one or two additional groups selected from alkoxy, alkoxyalkylcarbonyl, alkoxycarbonyl, alkylsulfanyl, C₂ alkynyl, arylalkoxycarbonyl, carboxy, cyano, cycloalkyl, halo, heterocyclyl, heterocyclylcarbonyl, hydroxy, and (NR^{e}R^{f})carbonyl; wherein the heterocyclyl is further optionally substituted with one, two, three, four, or five substituents independently selected from alkoxy, alkyl, cyano, halo, haloalkoxy, haloalkyl, and nitro.

The term “—NR^{e}R^{f},” as used herein, refers to two groups, R^{e} and R^{f}, which are attached to the parent molecular moiety through a nitrogen atom. R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR^{x}R^{y})alkyl, and (NR^{x}R^{y})carbonyl.

The term “—NR^{x}R^{y},” as used herein, refers to two groups, R^{x} and R^{y}, which are attached to the parent molecular moiety through a nitrogen atom. R^{x} and R^{y} are independently selected from hydrogen, alkoxycarbonyl, alkyl, alkylcarbonyl, unsubstituted aryl, unsubstituted arylalkoxycarbonyl, unsubstituted arylalkyl, unsubstituted cycloalkyl, unsubstituted heterocyclyl, and (NR^{x′}R^{y′})carbonyl, wherein R^{x′} and R^{y′} are independently selected from hydrogen and alkyl.

Asymmetric centers exist in the compounds of the present disclosure. These centers are designated by the symbols “R” or “S”, depending on the configuration of substituents around the chiral carbon atom. It should be understood that the disclosure encompasses all stereochemical isomeric forms, or mixtures thereof, which possess the ability to inhibit NS5A. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

The term “compounds of the present disclosure”, and equivalent expressions, are meant to embrace compounds of Formula (I), and pharmaceutically acceptable enantiomers, diastereomers, and salts thereof. Similarly, references to intermediates are meant to embrace their salts where the context so permits.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

The compounds of the present disclosure can exist as pharmaceutically acceptable salts. The term “pharmaceutically acceptable salt,” as used herein, represents salts or zwitterionic forms of the compounds of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use. The salts can be prepared during the final isolation and purification of the compounds or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, dihydrobromide, dihydrochloride, dihydroiodide, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, and N,N′-dibenzylethylenediamine. Other representative organic amines useful for the formation of base addition salts include ethylenediamine, ethanolamine, diethanolamine, piperidine, and piperazine.

When it is possible that, for use in therapy, therapeutically effective amounts of a compound of formula (I), as well as pharmaceutically acceptable salts thereof, may be administered as the raw chemical, it is possible to present the active ingredient as a pharmaceutical composition. Accordingly, the disclosure further provides pharmaceutical compositions, which include therapeutically effective amounts of compounds of formula (I) or pharmaceutically acceptable salts thereof, and one or more pharmaceutically acceptable carriers, diluents, or excipients. The term “therapeutically effective amount,” as used herein, refers to the total amount of each active component that is sufficient to show a meaningful patient benefit, e.g., a reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously. The compounds of formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the present disclosure there is also provided a process for the preparation of a pharmaceutical formulation including admixing a compound of formula (I), or a pharmaceutically acceptable salt thereof, with one or more pharmaceutically acceptable carriers, diluents, or excipients. The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

Pharmaceutical formulations may be presented in unit dose forms containing a predetermined amount of active ingredient per unit dose. Dosage levels of between about 0.01 and about 250 milligram per kilogram (“mg/kg”) body weight per day, preferably between about 0.05 and about 100 mg/kg body weight per day of the compounds of the present disclosure are typical in a monotherapy for the prevention and treatment of HCV mediated disease. Typically, the pharmaceutical compositions of this disclosure will be administered from about 1 to about 5 times per day or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Treatment may be initiated with small dosages substantially less than the optimum dose of the compound. Thereafter, the dosage is increased by small increments until the optimum effect under the circumstances is reached. In general, the compound is most desirably administered at a concentration level that will generally afford antivirally effective results without causing any harmful or deleterious side effects.

When the compositions of this disclosure comprise a combination of a compound of the present disclosure and one or more additional therapeutic or prophylactic agent, both the compound and the additional agent are usually present at dosage levels of between about 10 to 150%, and more preferably between about 10 and 80% of the dosage normally administered in a monotherapy regimen.

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research 1986, 3(6), 318.

Pharmaceutical formulations adapted for topical administration may be formulated as ointments, creams, suspensions, lotions, powders, solutions, pastes, gels, sprays, aerosols, or oils.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

The term “patient” includes both human and other mammals.

The term “treating” refers to: (i) preventing a disease, disorder or condition from occurring in a patient that may be predisposed to the disease, disorder, and/or condition but has not yet been diagnosed as having it; (ii) inhibiting the disease, disorder, or condition, i.e., arresting its development; and (iii) relieving the disease, disorder, or condition, i.e., causing regression of the disease, disorder, and/or condition.

The compounds of the present disclosure can also be administered with a cyclosporin, for example, cyclosporin A. Cyclosporin A has been shown to be active against HCV in clinical trials (Hepatology 2003, 38, 1282; Biochem. Biophys. Res. Commun. 2004, 313, 42; J. Gastroenterol. 2003, 38, 567).

Table 1 below lists some illustrative examples of compounds that can be administered with the compounds of this disclosure. The compounds of the disclosure can be administered with other anti-HCV active compounds in combination therapy, either jointly or separately, or by combining the compounds into a composition.

TABLE 1

| | | Type of | |
|---|---|---|---|
| | Physiological | Inhibitor or | Source |
| Brand Name | Class | Target | Company |
| NIM811 | | Cyclophilin | Novartis |
| | | Inhibitor | |
| Zadaxin | | Immuno- | Sciclone |
| | | modulator | |
| Suvus | | Methylene blue | Bioenvision |
| Actilon | | TLR9 agonist | Coley |
| (CPG10101) | | | |
| Batabulin | Anticancer | β-tubulin | Tularik Inc., |
| (T67) | | inhibitor | South San |
| | | | Francisco, CA |
| ISIS 14803 | Antiviral | antisense | ISIS |
| | | | Pharmaceuticals |
| | | | Inc, |
| | | | Carlsbad, |
| | | | CA/Elan |
| | | | Phamaceuticals |
| | | | Inc., New |
| | | | York, NY |
| Summetrel | Antiviral | antiviral | Endo |
| | | | Pharmaceuticals |
| | | | Holdings |
| | | | Inc., Chadds |
| | | | Ford, PA |
| GS-9132 (ACH- | Antiviral | HCV Inhibitor | Achillion/ |
| 806) | | | Gilead |
| Pyrazolopyrimidine | Antiviral | HCV Inhibitors | Arrow |
| compounds and | | | Therapeutics |
| salts | | | Ltd. |
| From WO- | | | |
| 2005047288 | | | |
| 26 May 2005 | | | |
| Levovirin | Antiviral | IMPDH inhibitor | Ribapharm |
| | | | Inc., Costa |
| | | | Mesa, CA |
| Merimepodib | Antiviral | IMPDH inhibitor | Vertex |
| (VX-497) | | | Pharmaceuticals |
| | | | Inc., |
| | | | Cambridge, MA |
| XTL-6865 | Antiviral | monoclonal | XTL |
| (XTL-002) | | antibody | Biopharmaceuticals |
| | | | Ltd., |
| | | | Rehovot, |
| | | | Isreal |
| Telaprevir | Antiviral | NS3 serine | Vertex |
| (VX-950, LY- | | protease | Pharmaceuticals |
| 570310) | | inhibitor | Inc., |
| | | | Cambridge, |
| | | | MA/Eli Lilly |
| | | | and Co. |
| | | | Inc., |
| | | | Indianapolis, |
| | | | IN |
| HCV-796 | Antiviral | NS5B Replicase | Wyeth/ |
| | | Inhibitor | Viropharma |
| NM-283 | Antiviral | NS5B Replicase | Idenix/ |
| | | Inhibitor | Novartis |
| GL-59728 | Antiviral | NS5B Replicase | Gene Labs/ |
| | | Inhibitor | Novartis |
| GL-60667 | Antiviral | NS5B Replicase | Gene Labs/ |
| | | Inhibitor | Novartis |
| 2′C MeA | Antiviral | NS5B Replicase | Gilead |
| | | Inhibitor | |
| PSI 6130 | Antiviral | NS5B Replicase | Roche |
| | | Inhibitor | |
| R1626 | Antiviral | NS5B Replicase | Roche |
| | | Inhibitor | |
| 2′C Methyl | Antiviral | NS5B Replicase | Merck |
| adenosine | | Inhibitor | |
| JTK-003 | Antiviral | RdRp inhibitor | Japan Tobacco |
| | | | Inc., Tokyo, |
| | | | Japan |
| Levovirin | Antiviral | ribavirin | ICN |
| | | | Pharmaceuticals, |
| | | | Costa |
| | | | Mesa, CA |
| Ribavirin | Antiviral | ribavirin | Schering- |
| | | | Plough |
| | | | Corporation, |
| | | | Kenilworth, |
| | | | NJ |
| Viramidine | Antiviral | Ribavirin | Ribapharm |
| | | Prodrug | Inc., Costa |
| | | | Mesa, CA |
| Heptazyme | Antiviral | ribozyme | Ribozyme |
| | | | Pharmaceuticals |
| | | | Inc., |
| | | | Boulder, CO |
| BILN-2061 | Antiviral | serine protease | Boehringer |
| | | inhibitor | Ingelheim |
| | | | Pharma KG, |
| | | | Ingelheim, |
| | | | Germany |
| SCH 503034 | Antiviral | serine protease | Schering |
| | | inhibitor | Plough |
| Zadazim | Immune | Immune | SciClone |
| | modulator | modulator | Pharmaceuticals |
| | | | Inc., San |
| | | | Mateo, CA |
| Ceplene | Immunomodulator | immune | Maxim |
| | | modulator | Pharmaceuticals |
| | | | Inc., San |
| | | | Diego, CA |
| CellCept | Immunosuppressant | HCV IgG immuno- | F. Hoffmann- |
| | | suppressant | La Roche LTD, |
| | | | Basel, |
| | | | Switzerland |
| Civacir | Immunosuppressant | HCV IgG immuno- | Nabi |
| | | suppressant | Biopharmaceuticals |
| | | | Inc., |
| | | | Boca Raton, |
| | | | FL |
| Albuferon - α | Interferon | albumin IFN-α2b | Human Genome |
| | | | Sciences |
| | | | Inc., |
| | | | Rockville, MD |
| Infergen A | Interferon | IFN | InterMune |
| | | alfacon-1 | Pharmaceuticals |
| | | | Inc., |
| | | | Brisbane, CA |
| Omega IFN | Interferon | IFN-ω | Intarcia |
| | | | Therapeutics |
| IFN-β and | Interferon | IFN-β and | Transition |
| EMZ701 | | EMZ701 | Therapeutics |
| | | | Inc., |
| | | | Ontario, |
| | | | Canada |
| Rebif | Interferon | IFN-β1a | Serono, |
| | | | Geneva, |
| | | | Switzerland |
| Roferon A | Interferon | IFN-α2a | F. Hoffmann- |
| | | | La Roche LTD, |
| | | | Basel, |
| | | | Switzerland |
| Intron A | Interferon | IFN-α2b | Schering- |
| | | | Plough |
| | | | Corporation, |
| | | | Kenilworth, |
| | | | NJ |
| Intron A and | Interferon | IFN-α2b/α1- | RegeneRx |
| Zadaxin | | thymosin | Biopharma. |
| | | | Inc., |
| | | | Bethesda, MD/ |
| | | | SciClone |
| | | | Pharmaceuticals |
| | | | Inc, San |
| | | | Mateo, CA |
| Rebetron | Interferon | IFN- | Schering- |
| | | α2b/ribavirin | Plough |
| | | | Corporation, |
| | | | Kenilworth, |
| | | | NJ |
| Actimmune | Interferon | INF-γ | InterMune |
| | | | Inc., |
| | | | Brisbane, CA |
| Interferon-β | Interferon | Interferon-β-1a | Serono |
| Multiferon | Interferon | Long lasting | Viragen/ |
| | | IFN | Valentis |
| Wellferon | Interferon | Lympho-blastoid | GlaxoSmithKline |
| | | IFN-αn1 | plc, |
| | | | Uxbridge, UK |
| Omniferon | Interferon | natural IFN-α | Viragen Inc., |
| | | | Plantation, |
| | | | FL |
| Pegasys | Interferon | PEGylated IFN- | F. Hoffmann- |
| | | α2a | La Roche LTD, |
| | | | Basel, |
| | | | Switzerland |
| Pegasys and | Interferon | PEGylated IFN- | Maxim |
| Ceplene | | α2a/ | Pharmaceuticals |
| | | immune | Inc., San |
| | | modulator | Diego, CA |
| Pegasys and | Interferon | PEGylated IFN- | F. Hoffmann- |
| Ribavirin | | α2a/ribavirin | La Roche LTD, |
| | | | Basel, |
| | | | Switzerland |
| PEG-Intron | Interferon | PEGylated IFN- | Schering- |
| | | α2b | Plough |
| | | | Corporation, |
| | | | Kenilworth, |
| | | | NJ |
| PEG-Intron/ | Interferon | PEGylated IFN- | Schering- |
| Ribavirin | | α2b/ribavirin | Plough |
| | | | Corporation, |
| | | | Kenilworth, |
| | | | NJ |
| IP-501 | Liver | antifibrotic | Indevus |
| | protection | | Pharmaceuticals |
| | | | Inc., |
| | | | Lexington, MA |
| IDN-6556 | Liver | caspase | Idun |
| | protection | inhibitor | Pharmaceuticals |
| | | | Inc., San |
| | | | Diego, CA |
| ITMN-191 (R- | Antiviral | serine protease | InterMune |
| 7227) | | inhibitor | Pharmaceuticals |
| | | | Inc., |
| | | | Brisbane, CA |
| GL-59728 | Antiviral | NS5B Replicase | Genelabs |
| | | Inhibitor | |
| ANA-971 | Antiviral | TLR-7 agonist | Anadys |
| Boceprevir | Antiviral | serine protease | Schering |
| | | inhibitor | Plough |
| TMS-435 | Antiviral | serine protease | Tibotec BVBA, |
| | | inhibitor | Mechelen, |
| | | | Belgium |
| BI-201335 | Antiviral | serine protease | Boehringer |
| | | inhibitor | Ingelheim |
| | | | Pharma KG, |
| | | | Ingelheim, |
| | | | Germany |
| MK-7009 | Antiviral | serine protease | Merck |
| | | inhibitor | |
| PF-00868554 | Antiviral | replicase | Pfizer |
| | | inhibitor | |
| ANA598 | Antiviral | Non-Nucleoside | Anadys |
| | | NS5B Polymerase | Pharmaceuticals, |
| | | Inhibitor | Inc., San |
| | | | Diego, CA, |
| | | | USA |
| IDX375 | Antiviral | Non-Nucleoside | Idenix |
| | | Replicase | Pharmaceuticals, |
| | | Inhibitor | Cambridge, |
| | | | MA, USA |
| BILB 1941 | Antiviral | NS5B Polymerase | Boehringer |
| | | Inhibitor | Ingelheim |
| | | | Canada Ltd |
| | | | R&D, Laval, |
| | | | QC, Canada |
| PSI-7851 | Antiviral | Nucleoside | Pharmasset, |
| | | Polymerase | Princeton, |
| | | inhibitor | NJ, USA |
| VCH-759 | Antiviral | NS5B Polymerase | ViroChem |
| | | Inhibitor | Pharma |
| VCH-916 | Antiviral | NS5B Polymerase | ViroChem |
| | | Inhibitor | Pharma |
| GS-9190 | Antiviral | NS5B Polymerase | Gilead |
| | | Inhibitor | |
| Peg- | Antiviral | Interferon | ZymoGenetics/ |
| interferon | | | Bristol-Myers |
| lamda | | | Squibb |

The compounds of the present disclosure may also be used as laboratory reagents. Compounds may be instrumental in providing research tools for designing of viral replication assays, validation of animal assay systems and structural biology studies to further enhance knowledge of the HCV disease mechanisms. Further, the compounds of the present disclosure are useful in establishing or determining the binding site of other antiviral compounds, for example, by competitive inhibition.

The compounds of this disclosure may also be used to treat or prevent viral contamination of materials and therefore reduce the risk of viral infection of laboratory or medical personnel or patients who come in contact with such materials, e.g., blood, tissue, surgical instruments and garments, laboratory instruments and garments, and blood collection or transfusion apparatuses and materials.

This disclosure is intended to encompass compounds having formula (I) when prepared by synthetic processes or by metabolic processes including those occurring in the human or animal body (in vivo) or processes occurring in vitro.

The abbreviations used in the present application, including particularly in the illustrative schemes and examples which follow, are well-known to those skilled in the art. Some of the abbreviations used are as follows: Boc or BOC for tert-butyoxycarbonyl; HATU for O-(7-azabenzotriazol-1-yl)-N,N,N′,N′-tetramethyluronium hexafluorophosphate; DIEA or DiPEA or DIPEA for diisopropylethylamine; NCS for N-chlorosuccinimide; NBS for N-bromosuccinimide; DMF for N,N-dimethylformamide; ACN or MeCN for acetonitrile; OAc for acetate; EtOAc for ethyl acetate; Et for ethyl; Bu for butyl; Ph for phenyl; Me for methyl; LDA for lithium diisopropylamide; Bn for benzyl; Ts for tosyl; RT or rt for room temperature or retention time (context will dictate); h or hr or hrs for hours; min or mins for minutes; DCM for dichloromethane; MeOH for methanol; d for days; THF for tetrahydrofuran; Et₂O for diethyl ether; Bz for benzoyl; AIBN for azobisisobutyronitrile; LiHMDS for lithium hexamethyldisilazide; Hex for hexanes; EDC for 1-ethyl-3-(3-dimethylaminopropyl) carbodiimide; DMAP for 4-N,N-dimethylaminopyridine; EtOH for ethanol; DEAD for diethyl azodicarboxylate; DMSO for dimethylsulfoxide; (S,S) Me-BPE-Rh for (−)-1,2-Bis((2S,5S)-2,5-dimethylphospholano)ethane(1,5-cyclooctadiene) rhodium(I) tetrafluoroborate; TBS for tert-butyldimethylsilyl; m-CPBA for metachloroperoxybenzoic acid; TBAF for tetrabutylammonium fluoride; DBU for 1,8-diazabicyclo[5.4.0]undec-7-ene; ON or o/n or on for overnight; AcOH for acetic acid; dppf for diphenylphosphinoferrocene; TFA for trifluoroacetic acid; pTsA or PTSA for paratoluenesulfonic acid; TMSCl for chlorotrimethylsilane; DDQ for 2,3-dichloro-5,6-dicyano-1,4-benzoquinone; Tf for trifluoromethylsulfonyl; TMSCN for trimethylsilyl cyanide; n-BuLi for n-butyllithium; and SEM for 2-trimethylsilylethoxymethoxy.

The compounds and processes of the present disclosure will be better understood in connection with the following synthetic schemes which illustrate the methods by which the compounds of the present disclosure may be prepared. Starting materials can be obtained from commercial sources or prepared by well-established literature methods known to those of ordinary skill in the art. It will be readily apparent to one of ordinary skill in the art that the compounds defined above can be synthesized by substitution of the appropriate reactants and agents in the syntheses shown below. It will also be readily apparent to one skilled in the art that the selective protection and deprotection steps, as well as the order of the steps themselves, can be carried out in varying order, depending on the nature of the variables to successfully complete the syntheses below. The variables are as defined above unless otherwise noted below.

Scheme A illustrates how key precursors A-1 and A-3 could be elaborated into an example of the target product A-7, and Scheme B through Scheme E highlight how these key precursors are prepared within the context of diverse heterocycle families.

### Scheme A

Standard acid catalyzed deprotection of carbamate A-1 followed by coupling with a protected amino acid such as Boc-Proline affords ketoamide A-2, which could be cyclized into imidazole A-5 by heating it in the presence of ammonium acetate. Alternatively, imidazole A-5 could be prepared from ketoester A-4, which in turn is prepared from haloketone A-3, by applying a similar thermal assisted reaction with ammonium acetate. Standard acid catalyzed deprotection of the Boc group followed by condensation with an acid under standard peptide coupling conditions such as HATU/DIEA affords A-7. Multiple approaches exist to prepare imidazole functionalized versions of A-7. For example, A-7 could be directly halogenated with reagents such as NCS or NBS; alternatively the halogenation of the imidazole moiety could be conducted before the Boc-deprotection step. It should be noted that in the case where the halogen moiety is either an iodide or a bromide, further functionalization is possible by applying metal-assisted coupling conditions such as the Suzuki-Miayura coupling that are well-established in the chemical literature.

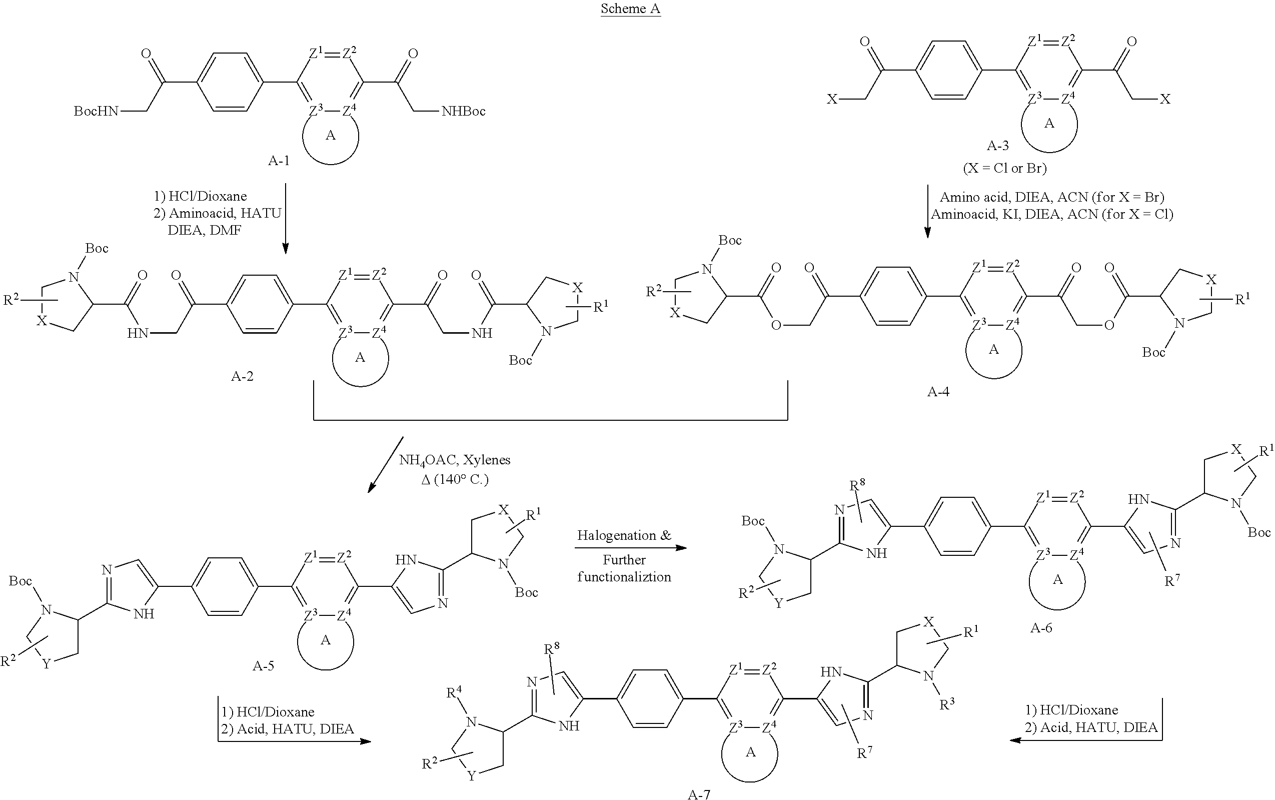

### Scheme B

Multiple approaches exist for the preparation of regioisomers B-6a and B-6b from benzoxazole B-1. Dibromide B-1 could be elaborated into diketone B-5 by employing a combination of Stille and Suzuki-Miayura coupling conditions, where the product from the Stille coupling step is treated with acid such as HCl to unveil the ketone moiety. Regioisomer separation could be conducted at the intermediate stage (either bromide B-4 or B-5) or the diketone stage B-6. Each diketone B-6 could be elaborated into dibromide B-7 by employing reagents such bromine 1n an alternative approach, bezoxazole B-1 could be coupled with boronic acid B-2 under Suzuki-Miayura condition, and the resultant regioisomeric mixture could be separated and individually elaborated to bromoketone B-6 by employing a combination of Stille coupling and in situ bromination with reagents such as NBS in the presence of water.

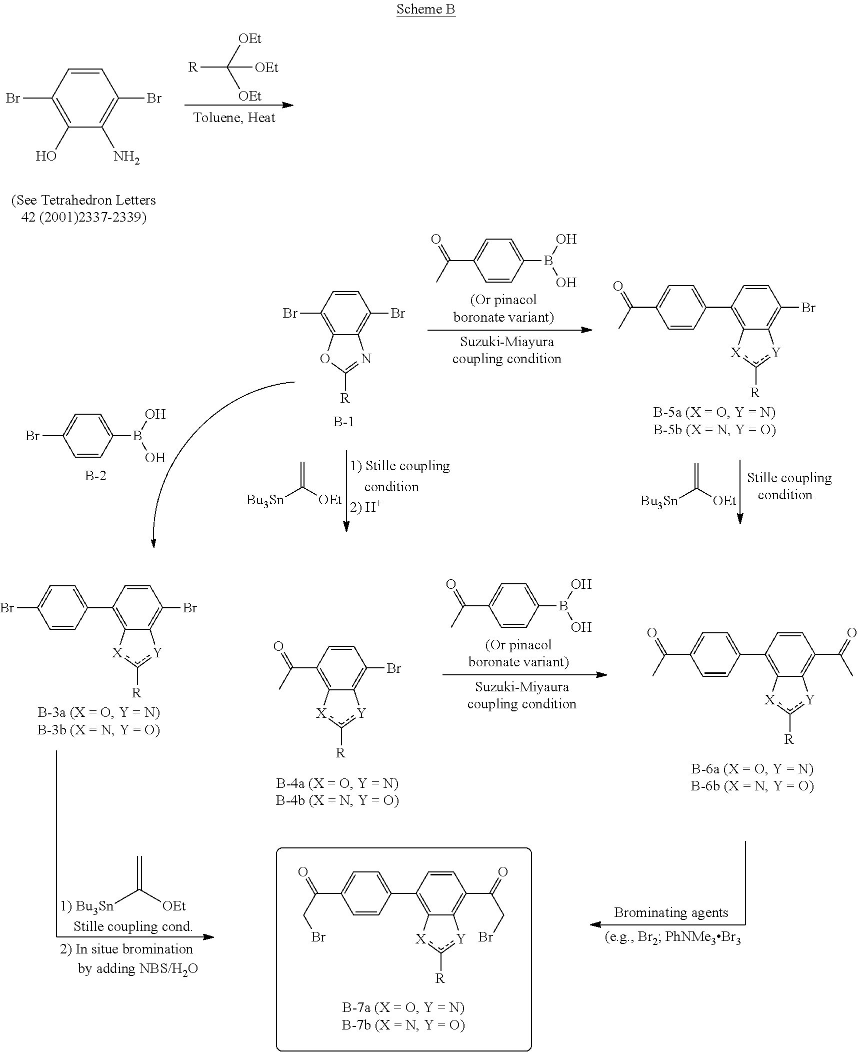

### Scheme C

Dibromide C-1 could be elaborated into bromoketone C-2a by employing the synthetic route discussed in Scheme B. If a complication arise during the final bromination step where the imidazole fragment of C-2 brominates competitively to afford C-2b, then it is possible to advance such a species through the imidazole construction step described in Scheme A, and then remove the bromide under reductive conditions (such as palladium/carbon-assisted hydrogenation). Alternatively, dibromide C-1 could be monolithiated and quenched with a Weinerb amide of Boc-glycine to afford regioisomeric bromides C-3. The separated regioisomers could be coupled with C-4 under Suzuki-Miayura condition to afford carbamate C-5.

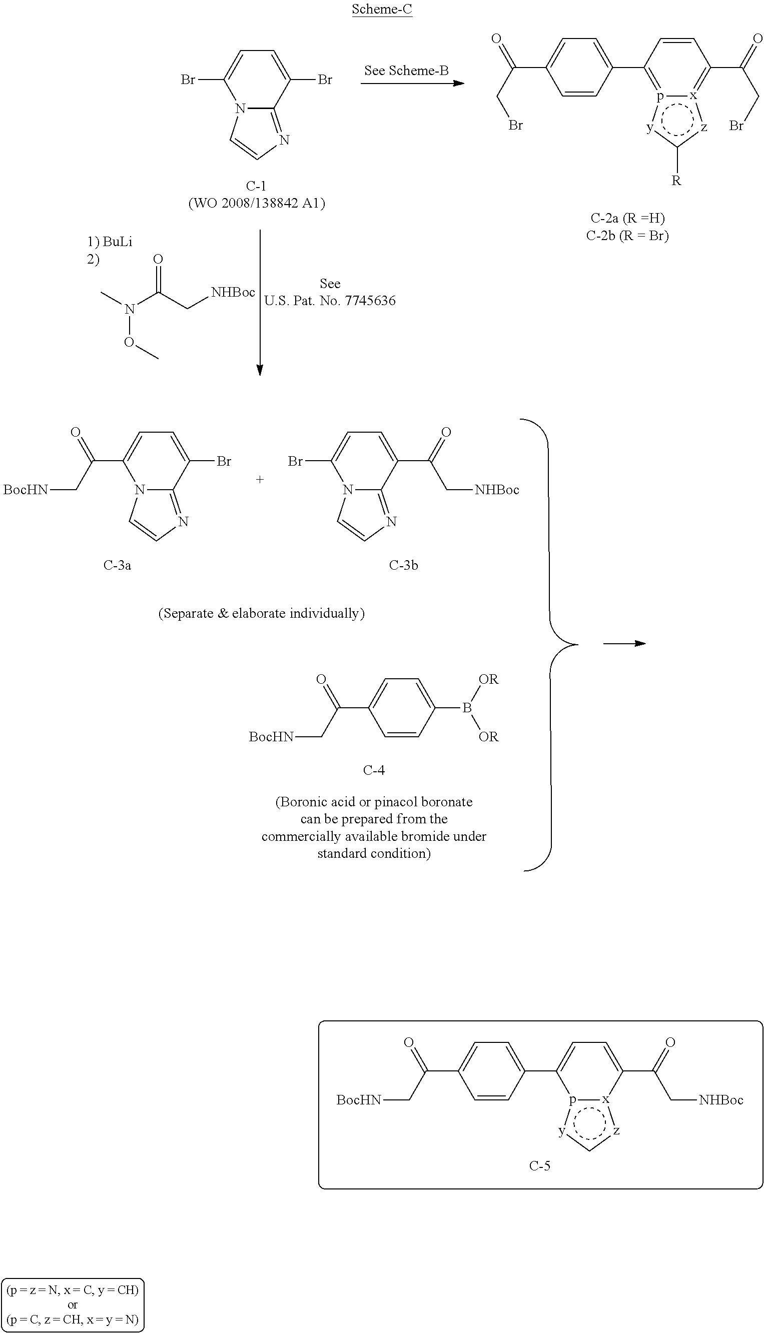

### Scheme D & Scheme E

In Scheme D, amine D-1 could be condensed with 2,5-dimethoxytetrahydrofuran under thermal condition, and then the resultant pyrrole could be reacted with methyl 2-chloro-2-oxoacetate, or any of its ester variants, to afford ketoester D-3. The removal of the Boc group under acid condition followed by in situ oxidation could afford diester D-6. Treatment of diester D-6 with CH₂ICl/LDA could afford chloroketone D-7. The preparation of the regioisomer of D-7 could be carried out according to the modified route described in Scheme E where methyl 4-(chlorocarbonyl)benzoate is employed in place of methyl 2-chloro-2-oxoacetate.

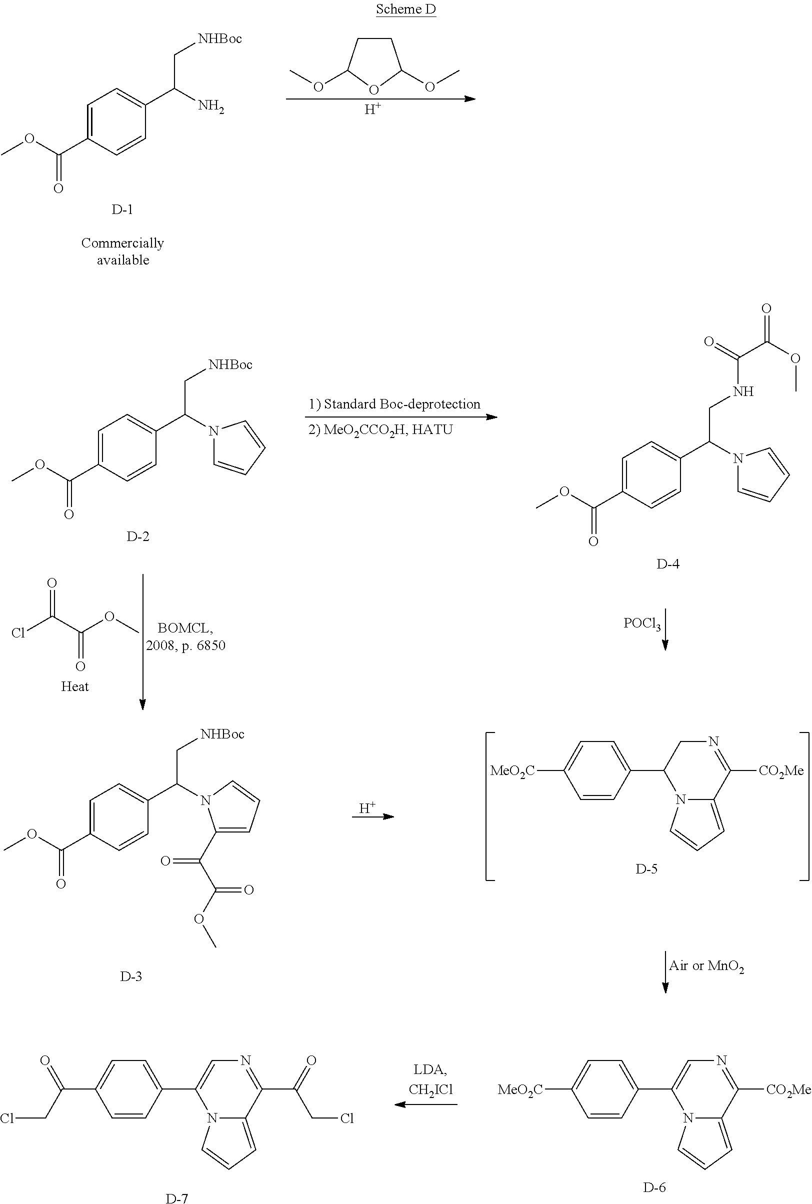

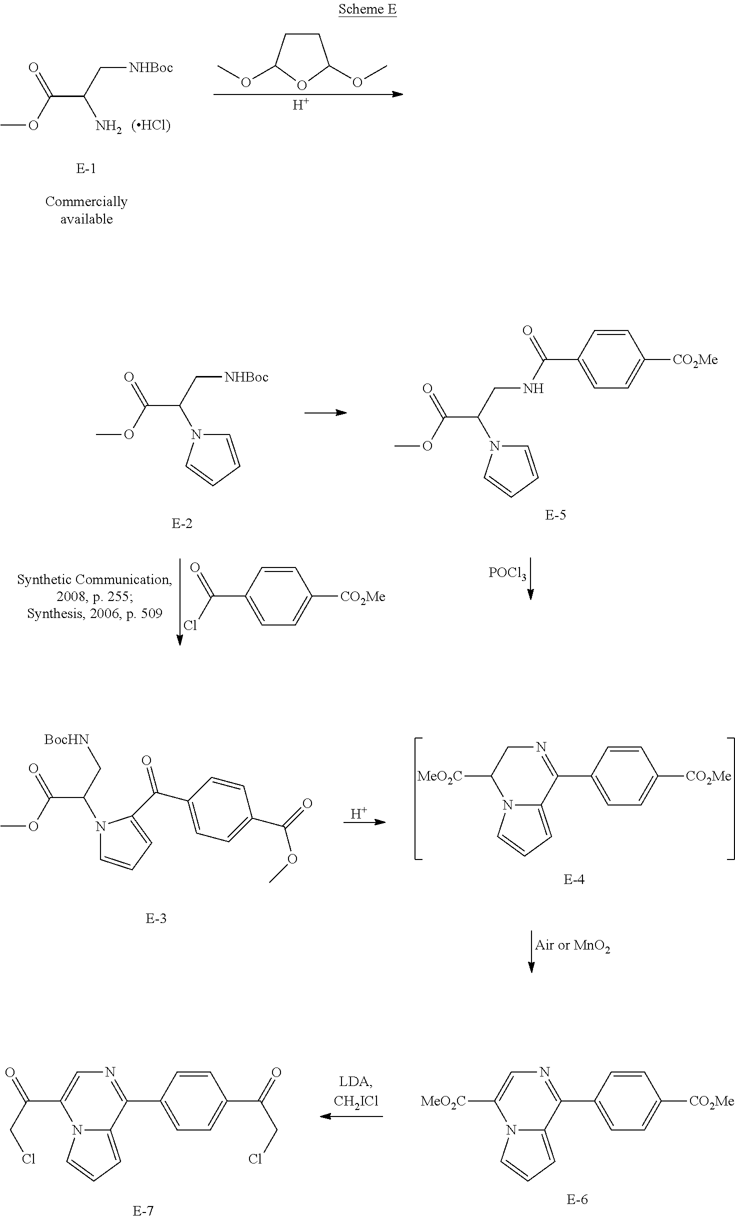

It would be apparent to one of ordinary skill in the art that the synthetic routes described in Schemes B through E would be equally applicable to the dihalogen precursors described in Figure A. It would also be apparent to one of ordinary skill in the art that modification of the routes described in Scheme A through E could be employed to prepare homodimeric variants of the final product as illustrated in Scheme F.

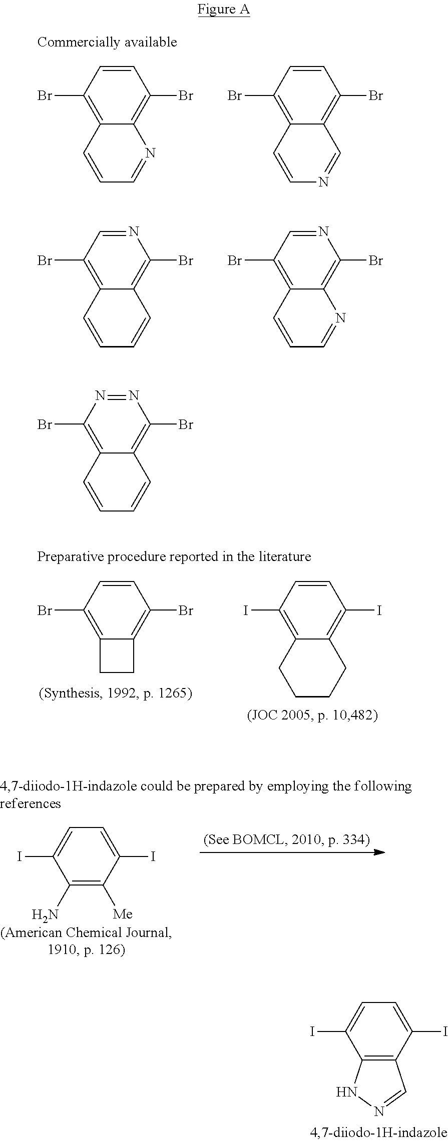

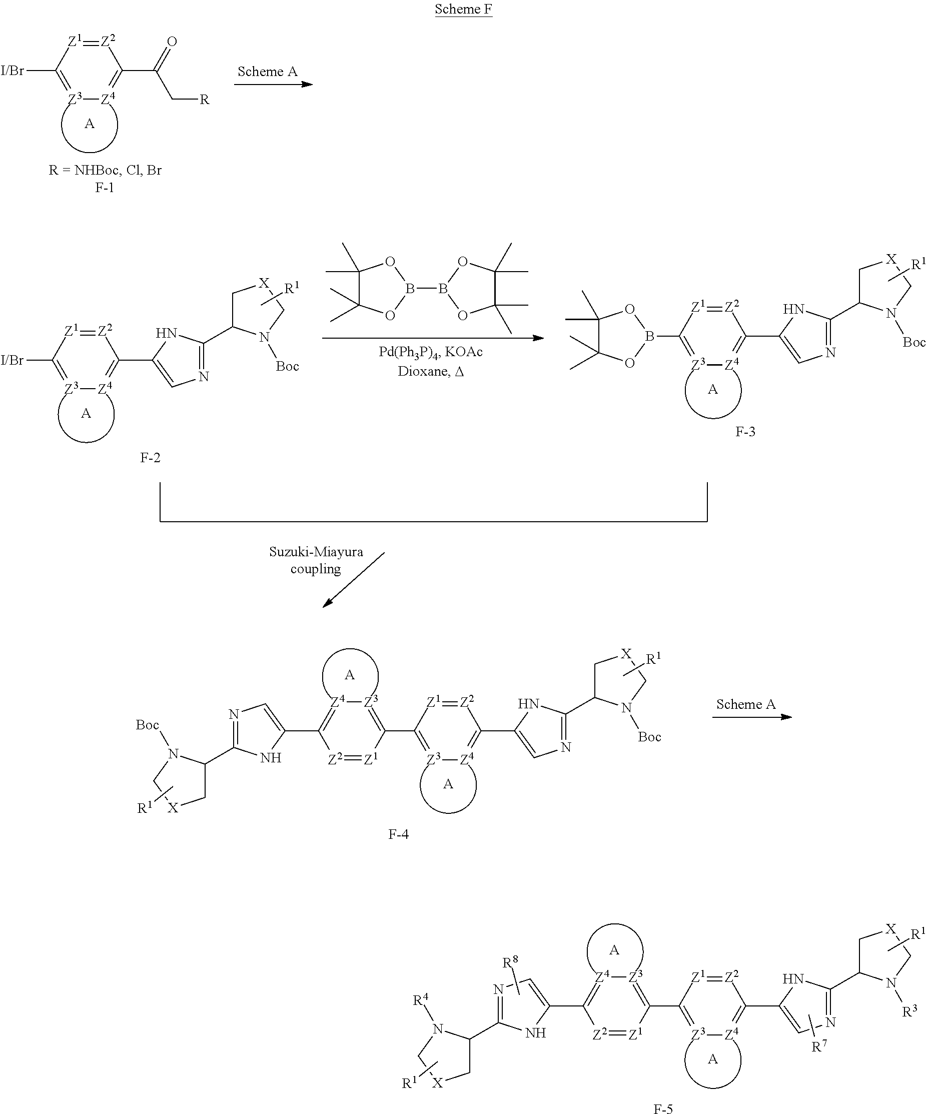

### Scheme 1

### Substituted Phenylglycine Derivatives

Substituted phenylglycine derivatives can be prepared by a number of methods shown below. Phenylglycine t-butyl ester can be reductively alkylated (pathway A) with an appropriate aldehyde and a reductant such as sodium cyanoborohydride in acidic medium. Hydrolysis of the t-butyl ester can be accomplished with strong acid such as HCl or trifluoroacetic acid. Alternatively, phenylglycine can be alkylated with an alkyl halide such as ethyl iodide and a base such as sodium bicarbonate or potassium carbonate (pathway B). Pathway C illustrates reductive alkylation of phenylglycine as in pathway A followed by a second reductive alkylation with an alternate aldehyde such as formaldehyde in the presence of a reducing agent and acid. Pathway D illustrates the synthesis of substituted phenylglycines via the corresponding mandelic acid analogs. Conversion of the secondary alcohol to a competent leaving group can be accomplished with p-toluensulfonyl chloride. Displacement of the tosylate group with an appropriate amine followed by reductive removal of the benzyl ester can provide substituted phenylglycine derivatives. In pathway E a racemic substituted phenylglycine derivative is resolved by esterification with an enantiomerically pure chiral auxiliary such as but not limited to (+)-1-phenylethanol, (−)-1-phenylethanol, an Evan's oxazolidinone, or enantiomerically pure pantolactone. Separation of the diastereomers is accomplished via chromatography (silica gel, HPLC, crystallization, etc) followed by removal of the chiral auxiliary providing enantiomerically pure phenylglycine derivatives. Pathway H illustrates a synthetic sequence which intersects with pathway E wherein the aforementioned chiral auxiliary is installed prior to amine addition. Alternatively, an ester of an arylacetic acid can be brominated with a source of bromonium ion such as bromine, N-bromosuccinimide, or CBr₄. The resultant benzylic bromide can be displaced with a variety of mono- or disubstituted amines in the presence of a tertiary amine base such as triethylamine or Hunig's base. Hydrolysis of the methyl ester via treatment with lithium hydroxide at low temperature or 6N HCl at elevated temperature provides the substituted phenylglycine derivatives. Another method is shown in pathway G. Glycine analogs can be derivatized with a variety of aryl halides in the presence of a source of palladium (0) such as palladium bis(tributylphosphine) and base such as potassium phosphate. The resultant ester can then be hydrolyzed by treatment with base or acid. It should be understood that other well known methods to prepare phenylglycine derivatives exist in the art and can be amended to provide the desired compounds in this description. It should also be understood that the final phenylglycine derivatives can be purified to enantiomeric purity greater than 98% ee via preparative HPLC.

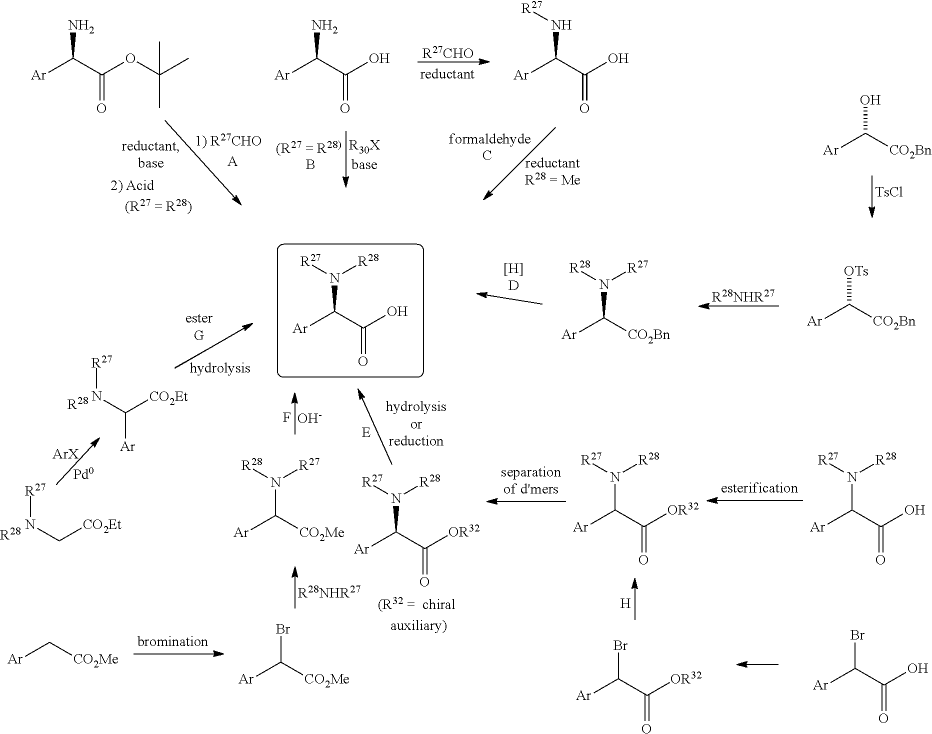

### Scheme 2

### Acylated Amino Acid Derivatives

In another embodiment of the present disclosure, acylated phenylglycine derivatives may be prepared as illustrated below. Phenylglycine derivatives wherein the carboxylic acid is protected as an easily removed ester, may be acylated with an acid chloride in the presence of a base such as triethylamine to provide the corresponding amides (pathway A). Pathway B illustrates the acylation of the starting phenylglycine derivative with an appropriate chloroformate while pathway C shows reaction with an appropriate isocyanate or carbamoyl chloride. Each of the three intermediates shown in pathways A-C may be deprotected by methods known by those skilled in the art (i.e.; treatment of the t-butyl ester with strong base such as HCl or trifluoroacetic acid).

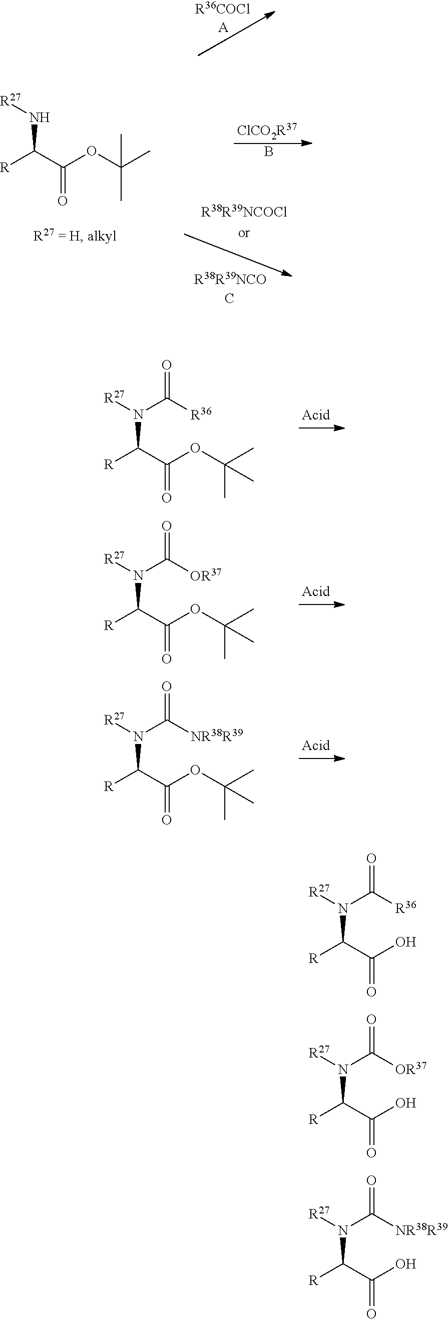

### Scheme 3

Amino-substituted phenylacetic acids may be prepared by treatment of a chloromethylphenylacetic acid with an excess of an amine

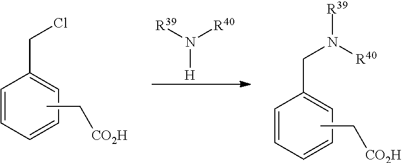

### Synthesis of Common Acid Precursors

For the synthesis of additional acid precursors besides the ones noted below, see Cap-1 to Cap-176 in WO 2011/059887.

### Cap-177a and Cap-177b

### Cap-177a and Cap-177b

### Step a

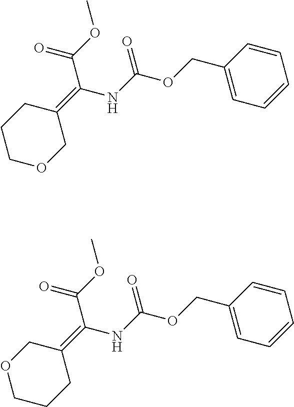

1,1,3,3-Tetramethylguanidine (0.985 mL, 7.85 mmol) was added to a stirred solution of methyl 2-(benzyloxycarbonylamino)-2-(dimethoxyphosphoryl)acetate (2.0 g, 6.0 mmol) in EtOAc (40 mL) and the mixture was stirred at rt under N₂ for 10 min. Then dihydro-2H-pyran-3(4H)-one (0.604 g, 6.04 mmol) was added and the mixture was stirred at rt for 16 h. The reaction mixture was then cooled in freezer for 10 min and neutralized with aq. citric acid (1.5 g in 20 mL water). The two phases were partitioned and the organic layer was washed with 0.25 N aq.HCl and brine, and then dried (MgSO₄) and concentrated to a colorless oil. The crude material was purified by flash silica chromatography (loading solvent: DCM, eluted with EtOAc/Hexanes, gradient from 20% to 30% EtOAc) to yield two isomeric products: The first eluted product was (Z)-methyl 2-(benzyloxycarbonylamino)-2-(2H-pyran-3(4H,5H,6H)-ylidene)acetate (490 mg) (white solid), and the second was (E)-methyl 2-(benzyloxycarbonylamino)-2-(2H-pyran-3(4H,5H,6H)-ylidene)acetate (433 mg) (white solid). LC-MS retention time 1.398 min (for Z-isomer) and 1.378 min (for E-isomer); m/z 304.08 (for Z-isomer) and 304.16 (for E-isomer) (MH—). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) (for Z-isomer) δ ppm 7.30-7.44 (m, 5H), 6.18 (br. s., 1H), 5.10-5.17 (m, 2H), 4.22 (s, 2H), 3.78 (br. s., 3H), 2.93-3.02 (m, 2 H), 1.80 (dt, J=11.7, 5.8 Hz, 2H), 1.62 (s, 2H). ¹H NMR (400 MHz, chloroform-d) (for E-isomer) δ ppm 7.31-7.44 (m, 5H), 6.12 (br. s., 1H), 5.13-5.17 (m, 2H), 4.64 (br. s., 2H), 3.70-3.82 (m, 5H), 2.49 (t, J=6.5 Hz, 2H), 1.80 (br. s., 2H). (Note: the absolute regiochemistry was determined by ¹H NMR shifts and coupling constants).

### Cap-177a and Cap-177b

### Step b

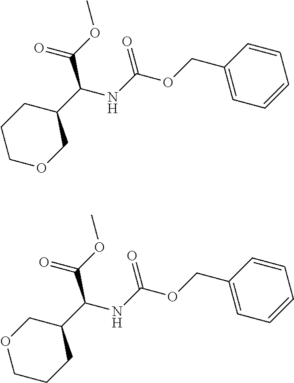

(−)-1,2-Bis((2S,5S)-2,5-dimethylphospholano)ethane(cyclooctadiene)-rhodium(I)tetrafluoroborate (28.2 mg, 0.051 mmol) was added to a stirred solution of (Z)-methyl 2-(benzyloxycarbonylamino)-2-(2H-pyran-3 (4H,5H,6H)-ylidene)acetate (310 mg, 1.015 mmol) in MeOH (10 mL) and the mixture was vacuum flushed with N₂, followed by H₂, and then the reaction was stirred under H₂ (60 psi) at rt for 2d. The reaction mixture was concentrated and the residue was purified by flash silica chromatography (loading solvent: DCM, eluted with 20% EtOAc in hexanes) to yield (S)-methyl 2-(benzyloxycarbonylamino)-2-((S)-tetrahydro-2H-pyran-3-yl)acetate (204 mg) as clear colorless oil. LC-MS retention time 1.437 min; m/z 307.89 (MH+). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) δ ppm 7.30-7.46 (m, 5H), 5.32 (d, J=8.8 Hz, 1H), 5.12 (s, 2H), 4.36 (dd, J=8.9, 5.6 Hz, 1H), 3.84-3.98 (m, 2H), 3.77 (s, 3H), 3.28-3.37 (m, 1H), 3.23 (dd, J=11.3, 10.5 Hz, 1H), 2.04-2.16 (m, 1H), 1.61-1.75 (m, 3H), 1.31-1.43 (m, 1H).

The other stereoisomer ((E)-methyl 2-(benzyloxycarbonylamino)-2-(2H-pyran-3(4H,5H,6H)-ylidene)acetate) (360 mg, 1.18 mmol) was reduced in a similar manner to yield (S)-methyl 2-(benzyloxycarbonylamino)-2-((R)-tetrahydro-2H-pyran-3-yl)acetate (214 mg) as clear colorless oil. LC-MS retention time 1.437 min; m/z 308.03 (MH+). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) δ ppm 7.30-7.44 (m, 5H), 5.31 (d, J=9.0 Hz, 1H), 5.12 (s, 2H), 4.31 (dd, J=8.7, 6.9 Hz, 1H), 3.80-3.90 (m, 2H), 3.77 (s, 3H), 3.37 (td, J=10.8, 3.5 Hz, 1H), 3.28 (dd, J=11.3, 9.8 Hz, 1H), 1.97-2.10 (m, 1H), 1.81 (d, J=11.5 Hz, 1H), 1.61-1.72 (m, 2H), 1.33-1.46 (m, 1H).

### Cap-177a and Cap-177b

### Step c

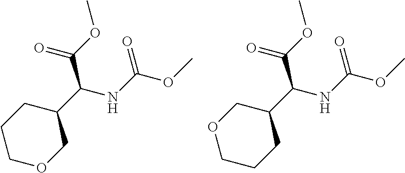

10% Pd/C (69.3 mg, 0.065 mmol) was added to a solution of (S)-methyl 2-(benzyloxycarbonylamino)-2-((S)-tetrahydro-2H-pyran-3-yl)acetate (200 mg, 0.651 mmol) and dimethyl dicarbonate [4525-33-1] (0.104 mL, 0.976 mmol) in MeOH (10 mL). The reaction mixture was vacuum flushed with N₂, followed by H₂, and then the reaction was stirred under H₂ (55 psi) at rt for 5 h. The reaction mixture was filtered through CELITE®/silica pad and the filtrate was concentrated to a colorless oil. The crude oil was purified by flash silica chromatography (loading solvent: DCM, eluted with 30% EtOAc in hexanes) to yield product (S)-methyl 2-(methoxycarbonylamino)-2-((S)-tetrahydro-2H-pyran-3-yl)acetate (132 mg) as colorless oil. LC-MS retention time 0.92 min; m/z 231.97 (MH+). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) δ ppm 5.24 (d, J=8.5 Hz, 1H), 4.34 (dd, J=8.9, 5.6 Hz, 1H), 3.84-3.97 (m, 2H), 3.77 (s, 3H), 3.70 (s, 3H), 3.29-3.38 (m, 1H), 3.23 (dd, J=11.2, 10.4 Hz, 1H), 2.03-2.14 (m, 1H), 1.56-1.75 (m, 3H), 1.32-1.43 (m, 1H).

Another diastereomer ((S)-methyl 2-(benzyloxycarbonylamino)-2-((R)-tetrahydro-2H-pyran-3-yl)acetate) was transformed in a similar manner to yield (S)-methyl 2-(methoxycarbonylamino)-2-((R)-tetrahydro-2H-pyran-3-yl)acetate as clear colorless oil. LC-MS retention time 0.99 min; m/z 231.90 (MH+). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) δ ppm 5.25 (d, J=8.0 Hz, 1H), 4.29 (dd, J=8.4, 7.2 Hz, 1H), 3.82-3.90 (m, 2H), 3.77 (s, 3H), 3.70 (s, 3H), 3.37 (td, J=10.8, 3.3 Hz, 1H), 3.28 (t, J=10.5 Hz, 1H), 1.96-2.08 (m, 1H), 1.81 (dd, J=12.9, 1.6 Hz, 1H), 1.56-1.72 (m, 2H), 1.33-1.46 (m, 1H).

### Cap-177a and Cap-177b

To a solution of (S)-methyl 2-(methoxycarbonylamino)-2-((S)-tetrahydro-2H-pyran-3-yl)acetate (126 mg, 0.545 mmol) in THF (4 mL) stirring at rt was added a solution of 1M LiOH (1.090 mL, 1.090 mmol) in water. The reaction was stirred at rt for 3 h, neutralized with 1M HCl (1.1 mL) and extracted with EtOAc (3×10 mL). The organics were dried, filtered and concentrated to yield (S)-2-(methoxycarbonylamino)-2-((S)-tetrahydro-2H-pyran-3-yl)acetic acid (Cap-177a) (125 mg) as a clear colorless oil. LC-MS retention time 0.44 min; m/z 218.00 (MH+). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) δ ppm 5.28 (d, J=8.8 Hz, 1H), 4.38 (dd, J=8.7, 5.6 Hz, 1H), 3.96-4.04 (m, 1H), 3.91 (d, J=11.0 Hz, 1H), 3.71 (s, 3H), 3.33-3.41 (m, 1H), 3.24-3.32 (m, 1H), 2.10-2.24 (m, 1H), 1.74-1.83 (m, 1H), 1.63-1.71 (m, 2H), 1.35-1.49 (m, 1H).

Another diastereomer ((S)-methyl 2-(methoxycarbonylamino)-2-((R)-tetrahydro-2H-pyran-3-yl)acetate) was transformed in a similar manner to yield (5)-2-(methoxycarbonylamino)-2-((R)-tetrahydro-2H-pyran-3-yl)acetic acid (Cap-177b) as clear colorless oil. LC-MS retention time 0.41 min; m/z 217.93 (MH+). LC data was recorded on a Shimadzu LC-10AS liquid chromatograph equipped with a PHENOMENEX® Luna 10u C18 3.0×50 mm column using a SPD-10AV UV-Vis detector at a detector wave length of 220 nM. The elution conditions employed a flow rate of 4 mL/min, a gradient of 100% Solvent A/0% Solvent B to 0% Solvent A/100% Solvent B, a gradient time of 3 min, a hold time of 1 min, and an analysis time of 4 min where Solvent A was 5% MeOH/95% H₂O/10 mM ammonium acetate and Solvent B was 5% H₂O/95% MeOH/10 mM ammonium acetate. MS data was determined using a MICROMASS® Platform for LC in electrospray mode. ¹H NMR (400 MHz, chloroform-d) δ ppm 6.18 (br. s., 1H), 5.39 (d, J=8.5 Hz, 1H), 4.27-4.37 (m, 1H), 3.82-3.96 (m, 2H), 3.72 (s, 3H), 3.42 (td, J=10.8, 3.3 Hz, 1H), 3.35 (t, J=10.4 Hz, 1H), 2.01-2.18 (m, 1H), 1.90 (d, J=11.8 Hz, 1H), 1.59-1.76 (m, 2H), 1.40-1.54 (m, 1H).

### Cap-178

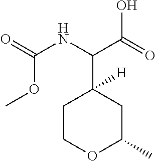

### Cap-178

### Step a

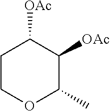

To a solution of (2S,3S,45)-2-methyl-3,4-dihydro-2H-pyran-3,4-diyl diacetate (5 g, 23.34 mmol) in 20 mL of MeOH in a hydrogenation tank was added Pd/C (150 mg, 0.141 mmol). The resulting mixture was hydrogenated at 40 psi on Parr Shaker for 1 hour. The mixture was then filtered and the filtrate was concentrated to afford Cap-178, step a (5.0 g) as a clear oil, which solidified while standing. ¹H NMR (500 MHz, CDCl₃) δ ppm 4.85-4.94 (1H, m), 4.69 (1H, t, J=9.46 Hz), 3.88-3.94 (1H, m), 3.44 (1H, td, J=12.21, 1.83 Hz), 3.36 (1H, dq, J=9.42, 6.12 Hz), 2.03-2.08 (1 H, m), 2.02 (3H, s), 2.00 (3H, s), 1.70-1.80 (1H, m), 1.16 (3H, d, J=6.10 Hz).

### Cap-178

### Step b

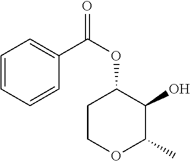

To a solution of Cap-178, step a (5.0 g, 23 mmol) in 50 mL of MeOH was added several drops of sodium methoxide. After stirring at room temperature for 30 min, sodium methoxide (0.1 mL, 23.12 mmol) was added and the solution was stirred at room temperature overnight. The solvent was then removed under vacuum. The residue was diluted with benzene and concentrated to afford the corresponding diol as a yellow solid. The solid was dissolved in 50 mL of pyridine and to this solution at −35° C. was added benzoyl chloride (2.95 mL, 25.4 mmol) dropwise. The resulting mixture was stirred at −35° C. for 1 hour then at room temperature overnight. The mixture was diluted with Et₂O and washed with water. The aqueous layer was extracted with EtOAc (2×). The combined organic layers were dried with MgSO₄ and concentrated. The crude product was purified by flash chromatography (silica gel, 5%-15% EtOAc/Hex) to afford Cap-178, step b (4.5 g) as clear oil which slowly crystallized upon prolonged standing. LC-MS: Anal. Calcd. for [M+Na]⁺ C₁₃H₁₆NaO₄ 259.09; found 259.0; ¹H NMR (500 MHz, CDCl₃) δ ppm 8.02-8.07 (2 H, m), 7.55-7.61 (1H, m), 7.45 (2H, t, J=7.78 Hz), 5.01 (1H, ddd, J=11.44, 8.70, 5.49 Hz), 3.98 (1H, ddd, J=11.90, 4.88, 1.53 Hz), 3.54 (1H, td, J=12.36, 2.14 Hz), 3.41 (1H, t, J=9.00 Hz), 3.31-3.38 (1H, m), 2.13-2.19 (1H, m), 1.83-1.94 (1H, m), 1.36 (3H, d, J=5.80 Hz).

### Cap-178

### Step c

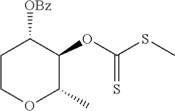

To a mixture of NaH (1.143 g, 28.6 mmol) (60% in mineral oil) in 6 mL of CS₂ was added Cap-178, step b (4.5 g, 19 mmol) in 40 mL of CS₂ dropwise over 15 min. The resulting mixture was stirred at room temperature for 30 min. The mixture turned light orange with some solid. MeI (14.29 mL, 229 mmol) was then added dropwise over 20 min. The mixture was then stirred at room temperature overnight. The reaction was carefully quenched with saturated NH₄Cl solution. The mixture was extracted with EtOAc (3×). The combined organic layers were dried with MgSO₄ and concentrated. The crude product was purified by flash chromatography (silica gel, 6% EtOAc/Hex) to afford Cap-178, step c (3.13 g) as clear oil. LC-MS: Anal. Calcd. for [M+Na]⁺ C₁₅H₁₈NaO₄S₂ 349.05; found 349.11; ¹HNMR (500 MHz, CDCl₃) δ ppm 7.94-8.00 (2H, m), 7.50-7.58 (1H, m), 7.41 (2H, t, J=7.78 Hz), 5.96 (1H, t, J=9.46 Hz), 5.28 (1H, ddd, J=11.37, 9.38, 5.49 Hz), 4.02 (1H, ddd, J=11.98, 4.96, 1.68 Hz), 3.54-3.68 (2H, m), 2.48 (3H, s), 2.31 (1H, dd), 1.88-1.99 (1H, m), 1.28 (3H, d).

### Cap-178

### Step d

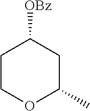

To a mixture of Cap-178, step c (3.13 g, 9.59 mmol) and AIBN (120 mg, 0.731 mmol) in 40 mL of benzene at 80° C. was added tri-n-butyltin hydride (10.24 mL, 38.4 mmol). The resulting mixture was stirred at reflux temperature for 20 min then cooled to room temperature. The mixture was diluted with diethyl ether and 100 mL of KF (10 g) aqueous solution was added and the mixture was stirred vigorously for 30 min. The two layers were then separated and the aqueous phase was extracted with EtOAc (2×). The organic layer was dried with MgSO₄ and concentrated. The crude product was purified by flash chromatography (silica gel, deactivated with 3% Et₃N in Hexanes and flushed with 3% Et₃N in Hexanes to remove tributyltin derivative and then eluted with 15% EtOAc/Hex) to afford Cap-178, step d (1.9 g) as clear oil. ¹H NMR (500 MHz, CDCl₃) δ ppm 7.98-8.07 (2H, m), 7.52-7.58 (1H, m), 7.43 (2H, t, J=7.63 Hz), 5.08-5.17 (1H, m), 4.06 (1H, ddd, J=11.90, 4.88, 1.53 Hz), 3.50-3.59 (2H, m), 2.08-2.14 (1H, m), 1.99-2.06 (1H, m), 1.69-1.80 (1H, m), 1.41-1.49 (1H, m), 1.24 (3H, d, J=6.10 Hz).

### Cap-178

### Step e

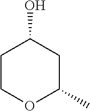

To a mixture of Cap-178, step d (1.9 g, 8.63 mmol) in 10 mL of MeOH was added sodium methoxide (2 mL, 4.00 mmol) (2 M in methanol). The resulting mixture was stirred at room temperature for 5 hours. The solvent was removed under vacuum. The mixture was neutralized with saturated NH₄Cl solution and extracted with EtOAc (3×). The organic layers were dried with MgSO₄ and concentrated to afford Cap-178, step e (0.8 g) as clear oil. The product was used in the next step without further purification. ¹H NMR (400 MHz, CDCl₃) δ ppm 4.01 (1H, ddd, J=11.80, 5.02, 1.76 Hz), 3.73-3.83 (1H, m), 3.36-3.46 (2H, m), 1.92-2.00 (1H, m), 1.88 (1H, m), 1.43-1.56 (1H, m), 1.23 (3H, d), 1.15-1.29 (1H, m).

### Cap-178

### Step f

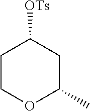

Tosyl-Cl (2.63 g, 13.77 mmol) was added to a solution of Cap-178, step e (0.8 g, 6.89 mmol) and pyridine (2.23 mL, 27.5 mmol) in 100 mL of CH₂Cl₂. The resulting mixture was stirred at room temperature for 3 days. 10 mL of water was then added into the reaction mixture and the mixture was stirred at room temperature for an hour. The two layers were separated and the organic phase was washed with water and 1 N HCl aq. solution. The organic phase was dried with MgSO₄ and concentrated to afford Cap-178, step f (1.75 g) as a light yellow solid. The product was used in the next step without further purification. Anal. Calcd. for [M+H]⁺ C₁₃H₁₉O₄S 271.10; found 270.90; ¹H NMR (500 MHz, CDCl₃) δ ppm 7.79 (2H, d, J=8.24 Hz), 7.34 (2H, d, J=7.93 Hz), 4.53-4.62 (1H, m), 3.94 (1H, ddd, J=12.13, 4.96, 1.83 Hz), 3.29-3.41 (2H, m), 2.45 (3H, s), 1.90-1.97 (1H, m), 1.79-1.85 (1 H, m), 1.64-1.75 (1H, m), 1.38-1.48 (1H, m), 1.17 (3H, d, J=6.10 Hz).

### Cap-178

### Step g

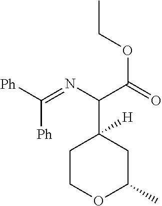

To a microwave tube was placed ethyl 2-(diphenylmethyleneamino)acetate (1.6 g, 5.92 mmol) and Cap-178, step f (1.6 g, 5.92 mmol). 10 mL of toluene was added. The tube was sealed and LiHMDS (7.1 mL, 7.10 mmol) (1N in toluene) was added dropwise under N₂. The resulting dark brown solution was heated at 100° C. under microwave radiation for 6 hours. To the mixture was then added water and the mixture was extracted with EtOAc (3×). The combined organic layers were washed with brine, dried with MgSO₄ and concentrated to afford a diastereomeric mixture of Cap-3, step g (3.1 g) as an orange oil. The crude mixture was submitted to the next step without separation. LC-MS: Anal. Calcd. for [M+H]⁺C₂₃H₂₈NO₃ 366.21; found 366.3.

### Cap-178

### Step h

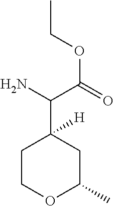

To a solution of the diastereomeric mixture of ethyl Cap-178, step g in 20 mL of THF was added HCl (30 mL, 60.0 mmol) (2 N aqueous). The resulting mixture was stirred at room temperature for 1 hour. The mixture was extracted with EtOAc and the aqueous layer was concentrated to afford an HCl salt of Cap-178, step h (1.9 g) as an orange oil. The salt was used in the next step without further purification. LC-MS: Anal. Calcd. for [M+H]⁺ C₁₀H₂₀NO₃ 202.14; found 202.1.

### Cap-178

### Step i

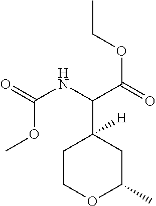

A solution of 1.9 g Cap-178, step h (HCl salt), DiPEA (4.19 mL, 24.0 mmol) and methyl chloroformate (1.24 mL, 16.0 mmol) in 20 mL of CH₂Cl₂ was stirred at room temperature for 1 hour. The mixture was diluted with CH₂Cl₂ and washed with water. The organic layer was dried with Na₂SO₄ and concentrated. The crude product was purified by flash chromatography (silica gel, 0-20% EtOAc/Hex) to afford Cap-178, step i (1.1 g) as a yellow oil. Anal. Calcd. for [M+Na]⁺ C₁₂H₂₁NNaO₅ 282.13; found 282.14; ¹H NMR (400 MHz, CDCl₃) δ ppm 5.16 (1H, br. s.), 4.43-4.58 (1H, m), 4.17-4.28 (2H, m), 3.89-4.03 (1H, m), 3.72-3.78 (2 H, m), 3.67-3.72 (3H, m), 2.07-2.19 (1H, m), 1.35-1.77 (4H, m), 1.30 (3H, td, J=7.09, 2.89 Hz), 1.19 (3H, d, J=6.53 Hz).

### Cap-178

### Step j

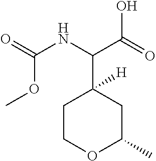

To a mixture of Cap-178, step i (1.1 g, 4.2 mmol) in 5 mL of THF and 2 mL of water was added LiOH (6.36 mL, 12.7 mmol) (2 N aq.). The resulting mixture was stirred at room temperature overnight. The mixture was then neutralized with 1 N HCl aq. and extracted with EtOAc (3×). The combined organic layers were dried with MgSO₄ and concentrated to afford Cap-178, step j (0.8 g) as a clear oil. LC-MS: Anal. Calcd. for [M+H]⁺ C₁₀H₁₈NO₅ 232.12; found 232.1; ¹H NMR (400 MHz, CDCl₃) δ ppm 5.20 (1H, d, J=8.28 Hz), 4.54 (1H, t, J=8.16 Hz), 3.95-4.10 (1H, m), 3.66-3.85 (5H, m), 2.15-2.29 (1H, m), 1.41-1.85 (4H, m), 1.23 (3H, dd, J=6.53, 1.76 Hz).

### Cap-178

### Step k

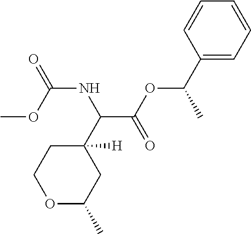

To a solution of Cap-178, step j (240 mg, 1.04 mmol), (S)-1-phenylethanol (0.141 mL, 1.142 mmol) and EDC (219 mg, 1.14 mmol) in 10 mL of CH₂Cl₂ was added DMAP (13.95 mg, 0.114 mmol). The resulting solution was stirred at room temperature overnight and the solvent was removed under vacuum. The residue was taken up into EtOAc, washed with water, dried with MgSO₄ and concentrated. The crude product was purified by chromatography (silica gel, 0-15% EtOAc/Hexanes) to afford Cap-178, step k as a mixture of two diastereomers. The mixture was separated by chiral HPLC(CHIRALPAK® AS column, 21×250 mm, 10 um) eluting with 90% 0.1% diethylamine/Heptane-10% EtOH at 15 mL/min to afford Cap-178, step k stereoisomer 1 (eluted first) and Cap-178, step k stereoisomer 2 (eluted second) as white solids. The stereochemistry of the isomers was not assigned.

Cap-178, step k stereoisomer 1 (130 mg): LC-MS: Anal. Calcd. for [M+Na]⁺ C₁₈H₂₅NNaO₅ 358.16; found 358.16; ¹H NMR (500 MHz, CDCl₃) δ ppm 7.28-7.38 (5H, m), 5.94 (1H, q, J=6.71 Hz), 5.12 (1H, d, J=9.16 Hz), 4.55 (1H, t, J=9.00 Hz), 3.72-3.81 (1H, m), 3.67 (3H, s), 3.60-3.70 (2H, m), 1.98-2.08 (1H, m), 1.59 (3H, d, J=6.71 Hz), 1.38-1.47 (2H, m), 1.30 (2H, t, J=5.34 Hz), 0.93 (3H, d, J=6.41 Hz).

### Cap-178

### Stereoisomer 1

To a solution of Cap-178, step k stereoisomer 1 ((S)-2-(methoxycarbonylamino)-2-((2S,4R)-2-methyltetrahydro-2H-pyran-4-yl)acetic acid) (150 mg, 0.447 mmol) in 10 mL of EtOH was added Pd/C (20 mg, 0.188 mmol) and the mixture was hydrogenated on Parr shaker at 40 psi overnight. The mixture was then filtered and the filtrate was concentrated to afford Cap-178, stereoisomer 1 (100 mg) as a sticky white solid. LC-MS: Anal. Calcd. for [M+H]⁺ C₁₀H₁₈NO₅ 232.12; found 232.1; ¹H NMR (500 MHz, CDCl₃) δ ppm 5.14-5.27 (1H, m), 4.51 (1H, t, J=8.39 Hz), 3.90-4.07 (1H, m), 3.60-3.83 (5H, m), 2.06-2.27 (1H, m), 1.45-1.77 (4H, m), 1.21 (3H, d, J=6.41 Hz).

### Cap-179 ((S)-Enantiomer or (R)-Enantiomer)

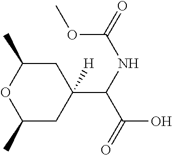

### Cap-179

### Step a

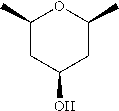

2,6-Dimethyl-4H-pyran-4-one (15 g, 121 mmol) was dissolved in ethanol (300 mL) and 10% Pd/C (1.28 g, 1.21 mmol) was added. The mixture was hydrogenated in a Parr shaker under H₂ (70 psi) at room temperature for 72 hrs. The reaction mixture was filtered through a pad of diatomaceous earth (Celite®) and washed with ethanol. The filtrate was concentrated in vacuum and the residue was purified via flash chromatography (10% to 30% EtOAc/Hex). Two fractions of clear oils were isolated. The first eluting fractions were a mixture of (2R,4r,6S)-2,6-dimethyltetrahydro-2H-pyran-4-ol (Cap-1, step a) and (2R,4s,6S)-2,6-dimethyltetrahydro-2H-pyran-4-ol (1.2 g) while the latter eluting fractions corresponded to only Cap-179, step a (10.73 g). ¹H NMR (500 MHz, CDCl₃) δ ppm 3.69-3.78 (1H, m), 3.36-3.47 (2H, m), 2.10 (1H, br. s.), 1.88 (2H, dd, J=12.05, 4.73 Hz), 1.19 (6H, d, J=6.10 Hz), 1.10 (2H, q, J=10.70 Hz). ¹³C NMR (126 MHz, CDCl₃) δ ppm 71.44 (2 C), 67.92 (1 C), 42.59 (2 C), 21.71 (2 C).

### Cap-179

### Step b

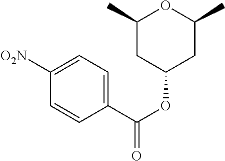

DEAD (166 mL, 330 mmol) was added drop wise to a solution of Cap-179, step a (10.73, 82 mmol), 4-nitrobenzoic acid (48.2 g, 288 mmol) and Ph₃P (86 g, 330 mmol) in benzene (750 mL). Heat evolution was detected and the resulting amber solution was stirred at ambient temperature for 18 h. Solvent was removed under reduced pressure and the residue was triturated with Et₂O (200 mL) to remove triphenylphosphine oxide (10 g). The remaining mixture was purified via Biotage® (0 to 5% EtOAc/Hex; 300 g column X 4). A white solid corresponding to Cap-179, step b (19.36 g) was isolated. ¹H NMR (500 MHz, CDCl₃) δ ppm 8.27-8.32 (2H, m), 8.20-8.24 (2H, m), 5.45 (1H, quin, J=2.82 Hz), 3.92 (2H, dqd, J=11.90, 6.10, 1.53 Hz), 1.91 (2H, dd, J=14.80, 2.29 Hz), 1.57 (2H, dt, J=14.65, 3.05 Hz), 1.22 (6H, d, J=6.10 Hz). ¹³C NMR (126 MHz, CDCl₃) δ ppm 163.81 (1 C), 150.55 (1 C), 135.94 (1 C), 130.64 (2 C), 123.58 (2 C), 70.20 (1 C), 68.45 (2 C), 36.95 (2 C), 21.84 (2 C). LC-MS: Anal. Calcd. for [M]⁺ C₁₄H₁₂NO₅: 279.11; found 279.12.

### Cap-179

### Step c

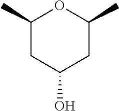

A solution of LiOH (8.30 g, 347 mmol) in water (300 mL) was added to a solution of Cap-179, step b (19.36 g, 69.3 mmol) in THF (1000 mL) and the resulting mixture was stirred at ambient temperature for 16 h. THF was removed under reduced pressure and the aqueous layer was diluted with more water (200 mL) and extracted with EtOAc (3×200 mL). The combined organic layers were dried (MgSO₄), filtered and concentrated under vacuum. An oily residue with a white solid was recovered. The mixture was triturated with hexanes and the solid was removed by filtration to yield a clear oil corresponding to Cap-179, step c (8.03 g). ¹H NMR (500 MHz, CDCl₃) δ ppm 4.21 (1H, quin, J=2.82 Hz), 3.87-3.95 (2H, m), 1.72 (1H, br. s.), 1.63 (2H, dd, J=14.34, 2.14 Hz), 1.39-1.47 (2H, m), 1.17 (6H, d, J=6.41 Hz). ¹³C NMR (126 MHz, CDCl₃) δ ppm 67.53 (2 C), 64.71 (1 C), 39.99 (2C), 21.82 (2 C).

### Cap-179

### Step d

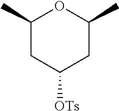

p-Tosyl chloride (23.52 g, 123 mmol) was added to a solution of Cap-179, step c (8.03 g, 61.7 mmol) and pyridine (19.96 mL, 247 mmol) in CH₂Cl₂ (750 mL) at room temperature and stirred for 36 h. As the reaction did not proceed to completion, CH₂Cl₂ was removed under reduced pressure and stirring continued for another 48 h. The mixture was then added to CH₂Cl₂ (100 mL) and water (100 mL) and stirred at ambient temperature for 2 h. The mixture was separated and the organic layer was the washed thoroughly with 1N aq. HCl (2×50 mL). The organic layer was then dried (MgSO₄), filtered and concentrated. A yellow oil corresponding to Cap-179, step d (14.15 g) was isolated, which solidified under vacuum as an off-white solid. ¹H NMR (500 MHz, CDCl₃) δ ppm 7.80 (2H, d, J=8.24 Hz), 7.35 (2H, d, J=7.93 Hz), 4.88 (1H, quin, J=2.82 Hz), 3.79-3.87 (2H, m), 2.46 (3H, s), 1.76 (2H, dd, J=14.50, 2.59 Hz), 1.36 (2H, ddd, J=14.34, 11.60, 2.75 Hz), 1.12 (6H, d, J=6.10 Hz). ¹³C NMR (126 MHz, CDCl₃) δ ppm 144.64 (1 C), 134.24 (1 C), 129.82 (2 C), 127.61 (2 C), 77.34 (1 C), 67.68 (2 C), 37.45 (2 C), 21.61 (1 C), 21.57 (2 C). LC-MS: Anal. Calcd. for [2M+H]⁺ C₂₈H₄₁O₈S₂: 569.22; found 569.3.

### Cap-179

### Step e

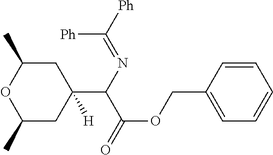

LiHMDS (29.7 mL, 29.7 mmol, 1 M in THF) was added to a solution of Cap-179, step d (7.05 g, 24.8 mmol) and benzyl 2-(diphenylmethyleneamino)acetate (8.57 g, 26.0 mmol) in toluene (80 mL) at room temperature in a pressure tube and the resulting mixture was then stirred for 5 h at 100° C. The reaction was quenched with water (100 mL), extracted with EtOAc, washed with water, dried over MgSO₄, filtrated, and concentrated in vacuum. The residue was purified via Biotage® (0% to 15% EtOAc/Hex; 240 g column) and a yellow oil corresponding to Cap-179, step e (8.76 g) was isolated as a racemic mixture. ¹H NMR (400 MHz, CDCl₃) δ ppm 7.62-7.71 (2H, m), 7.30-7.45 (11H, m), 7.05 (2H, dd, J=7.65, 1.63 Hz), 5.13-5.22 (2 H, m), 3.89 (1H, d, J=6.78 Hz), 3.46 (2H, dquind, J=11.27, 5.90, 2.01 Hz), 2.34-2.45 (1H, m), 1.58-1.66 (1H, m), 1.34-1.43 (1H, m), 1.19 (3H, d, J=6.02 Hz), 1.03-1.16 (4H, m), 0.83-0.97 (1H, m). ¹³C NMR (101 MHz, CDCl₃) δ ppm 170.84 (1 C), 170.68 (1 C), 139.01 (1 C), 135.96 (1 C), 135.51 (1 C), 130.04 (1 C), 128.49 (2 C), 128.20 (1 C), 128.09 (4 C), 127.97 (2 C), 127.85 (1 C), 127.67 (2 C), 127.47 (2 C), 72.76 (1 C), 72.46 (1 C), 69.77 (1 C), 65.99 (1 C), 39.11 (1 C), 35.90 (1 C), 35.01 (1 C), 21.74 (1 C), 21.65 (1 C). LC-MS Anal. Calcd. for [2M+Na]⁺ C₅₈H₆₂N₂NaO₆: 905.45; found 905.42.

### Cap-179

### Step f

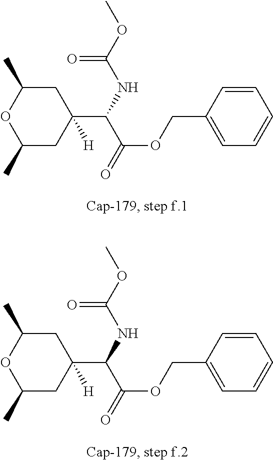

Cap-179, step e (8.76 g, 19.84 mmol) was dissolved in THF (100 mL) and treated with 2 N HCl in water (49.6 mL, 99 mmol). The resulting clear solution was stirred at ambient temperature for 4 h and then THF was removed under reduced pressure. The remaining aqueous layer was extracted with EtOAc (3×30 mL) and concentrated under vacuum, to afford the corresponding crude amine. The residue was taken up in CH₂Cl₂ (100 mL) and charged with DIEA (11.8 mL, 67.6 mmol) and methyl chloroformate (1.962 mL, 25.3 mmol). The resulting solution was stirred at ambient temperature for 2 h. The reaction mixture was diluted with CH₂Cl₂ (50 mL) and washed with water (100 mL) and brine (100 mL). The organic layer was dried (MgSO₄), filtered and concentrated. The residue was purified via Biotage® (15% to 25% EtOAc/Hex; 80 g column). A clear colorless oil corresponding to racemic Cap-179, step f (5.27 g) was recovered. ¹H NMR (400 MHz, CDCl₃) δ ppm 7.32-7.41 (5 H, m), 5.13-5.28 (3H, m), 4.36 (1H, dd, J=8.16, 4.64 Hz), 3.69 (3H, s), 3.30-3.47 (2H, m), 2.00-2.16 (1H, m), 1.52 (1H, d, J=12.55 Hz), 1.33 (1H, d, J=12.30 Hz), 1.15 (6H, dd, J=6.02, 5.02 Hz), 0.88-1.07 (2H, m). ¹³C NMR (101 MHz, CDCl₃) δ ppm 171.39 (1 C), 156.72 (1 C), 135.20 (2 C), 128.60 (2 C), 128.57 (1 C), 128.52 (2 C), 72.77 (1 C), 72.74 (1 C), 67.16 (1 C), 57.81 (1 C), 52.40 (1 C), 38.85 (1 C), 35.56 (1 C), 34.25 (1 C), 21.94 (2 C). LC-MS: Anal. Calcd. for [M+H]⁺ C₁₈H₂₆NO_{5: 336.18}; found 336.3.

A chiral method was developed to separate the racemic mixture by using 20% ethanol as the modifier on a CHIRALPAK® AS-H column (50×500 mm, 20 μm) (Wavelength=220 nm, Flow rate=100 mL/min for 22 min, Solvent A=0.1% diethylamine in heptanes, Solvent B=EtOH). The two separated isomers, corresponded to (S)-benzyl 2-((2R,4r,6S)-2,6-dimethyltetrahydro-2H-pyran-4-yl)-2-((methoxycarbonyl)amino)acetate (Cap-179, step f.1) (Rt=9.8 min, 2.2 g) and (R)-benzyl 2-((2R,4r,6S)-2,6-dimethyltetrahydro-2H-pyran-4-yl)-2-((methoxycarbonyl)amino)acetate (Cap-179, step f.2) (Rt=16.4 min, 2.1 g) and they each exhibited the same analytical data as the corresponding mixture (see above).

### Cap-179 [(S)-Enantiomer]

(S)-benzyl 2-((2R,4r,6S)-2,6-dimethyltetrahydro-2H-pyran-4-yl)-2-((methoxycarbonyl)amino)acetate (Cap-179, step f.1) (2.2 g, 6.6 mmol) was dissolved in MeOH (50 mL) in a Parr bottle and charged with 10% Pd/C (0.349 g, 0.328 mmol). The suspension was then placed in a Parr shaker and the mixture was flushed with N₂ (3×), placed under 40 psi of H₂ and shaken at room temperature for 15 h. The catalyst was filtered off through a pad of diatomaceous earth (Celite®) and the solvent was removed under reduced pressure, to yield an amber solid corresponding to (S)-Cap-179 (1.6 g). ¹H NMR (500 MHz, DMSO-d₆) δ ppm 12.74 (1H, br. s.), 7.35 (1H, d, J=6.10 Hz), 3.85 (1H, br. s.), 3.53 (3H, s), 3.35 (2H, ddd, J=15.95, 9.99, 6.10 Hz), 1.97 (1H, br. s.), 1.48 (2H, t, J=13.28 Hz), 1.06 (6H, d, J=6.10 Hz), 0.82-1.00 (2H, m). ¹³C NMR (101 MHz, DMSO-d₆) δ ppm 176.93 (1 C), 156.72 (1 C), 72.10 (1 C), 71.92 (1 C), 58.54 (1 C), 51.35 (1 C), 36.88 (1 C), 35.82 (1 C), 34.71 (1 C), 21.90 (2 C). Note: The absolute stereochemical assignment was made by single crystal X-ray analysis of (S)-phenethanol ester derivative. Cap-179 [(R)-enantiomer] was prepared similarly: ¹H NMR (500 MHz, DMSO-d₆) δ ppm 12.50 (1H, br. s.), 7.31 (1H, br. s.), 3.84 (1H, t, J=7.32 Hz), 3.53 (3H, s), 3.29-3.41 (2H, m), 1.99 (1H, s), 1.48 (2H, t, J=14.34 Hz), 1.06 (6H, d, J=6.10 Hz), 0.95 (1H, q, J=12.21 Hz), 0.87 (1H, q, J=11.80 Hz). [Note: the minor variation in the ¹H NMR profile of the enantiomers is likely a result of a difference in sample concentration.]

### Cap-180

### Racemic Mixture

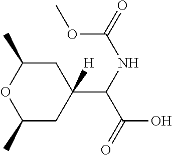

### Cap-180

### Step a

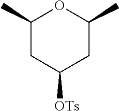

p-Tosyl-Cl (4.39 g, 23.0 mmol) was added to a solution of Cap-179, step a (1.50 g, 11.5 mmol) and pyridine (3.73 mL, 46.1 mmol) in CH₂Cl₂ (50 mL) at room temperature and stirred for 2 days. The reaction was diluted with CH₂Cl₂, washed with water, then 1 N HCl. The organic layer was dried (MgSO₄) and concentrated to a yellow oil which was purified via BIOTAGE® (5% to 20% EtOAc/Hex; 40 g column). A clear oil that solidified under vacuum and corresponding to Cap-180, step a (2.89 g) was isolated. LC-MS: Anal. Calcd. for [2M+Na]⁺ C₂₈H₄₀NaO₈S₂: 591.21; found 591.3. ¹H NMR (500 MHz, CDCl₃) δ ppm 7.80 (2H, d, J=8.24 Hz), 7.35 (2H, d, J=7.93 Hz), 4.59 (1H, tt, J=11.37, 4.96 Hz), 3.36-3.46 (2H, m), 2.46 (3H, s), 1.91 (2H, dd, J=12.05, 5.04 Hz), 1.37 (2H, dt, J=12.67, 11.52 Hz), 1.19 (6 H, d, J=6.10 Hz).

### Cap-180

### Step b

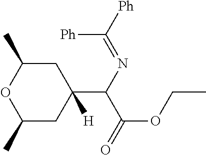

LiHMDS 1 N (7.09 mL, 7.09 mmol) was added to a solution of Cap-180, step a (1.68 g, 5.91 mmol) and ethyl 2-(diphenylmethyleneamino)acetate (1.579 g, 5.91 mmol) in toluene (30 mL) at room temperature and the resulting mixture was then stirred for 16 h at 85° C. The reaction was quenched with water (50 mL), extracted with EtOAc, washed with water, dried over MgSO₄, filtrated, and concentrated in vacuo. The residue was purified via BIOTAGE® (0% to 15% EtOAc/Hex; 40 g column). A clear yellowish oil corresponding to Cap-180, step b (racemic mixture; 0.64 g) was isolated. LC-MS: Anal. Calcd. for [M+H]⁺ C₂₄H₃₀NO₃: 380.22; found 380.03. ¹H NMR (400 MHz, CDCl₃) δ ppm 7.64-7.70 (2H, m), 7.45-7.51 (3H, m), 7.38-7.44 (1H, m), 7.31-7.37 (2H, m), 7.13-7.19 (2H, m), 4.39 (1H, d, J=10.54 Hz), 4.16-4.26 (2H, m), 3.29-3.39 (1H, m), 2.93-3.03 (1H, m), 2.70 (1 H, m, J=9.41, 4.14 Hz), 1.42-1.49 (2H, m), 1.31-1.37 (1H, m), 1.29 (4H, t, J=7.15 Hz), 1.04 (6H, dd, J=7.78, 6.27 Hz).

### Cap-180

### Step c

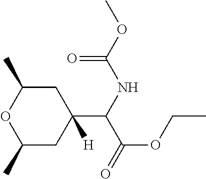

Cap-180, step b (0.36 g, 0.949 mmol) was dissolved in THF (10 mL) and treated with 2 N HCl (1.897 mL, 3.79 mmol). The resulting clear solution was stirred at ambient temperature for 20 h and THF was removed under reduced pressure. The remaining aqueous layer was extracted with hexanes (3×20 mL) and after diluting with H₂O (20 mL), the aqueous phase was basified with 1 N NaOH to pH=10 and extracted with EtOAc (3×10 mL). The combined organic layers were dried (MgSO₄), filtered and concentrated under vacuum. The resulting residue was taken up in CH₂Cl₂ (10.00 mL) and charged with DIEA (0.497 mL, 2.85 mmol) and methyl chloroformate (0.081 mL, 1.044 mmol). The resulting solution was stirred at ambient temperature for 2 h and the reaction mixture was quenched with water (10 mL) and the organic layer was removed under reduced pressure. Aqueous layer was extracted with EtOAc (3×10 mL) and the combined organic layers were dried (MgSO₄), filtered and concentrated. An amber oil corresponding to Cap-180, step c (0.21 g) was recovered and it was used without further purification. LC-MS: Anal. Calcd. for [M+H]⁺ C₁₃H₂₄NO₅: 273.17; found 274.06. ¹H NMR (400 MHz, CDCl₃) δ ppm 5.20 (1H, d, J=8.03 Hz), 4.59 (1H, t, J=10.16 Hz), 4.11-4.27 (3H, m), 3.69-3.82 (2H, m), 3.64 (3H, s), 1.95-2.07 (1H, m), 1.63 (1H, d, J=13.80 Hz), 1.41 (2H, dd, J=8.03, 4.02 Hz), 1.31-1.37 (1H, m), 1.26 (3H, t, J=7.15 Hz), 1.16 (1H, d, J=6.27 Hz), 1.12 (6H, dd, J=6.15, 3.89 Hz).

### Cap-180

### Racemic Mixture

Cap-180, step c (0.32 g, 1.2 mmol) was dissolved in THF (10 mL) and charged with LiOH (0.056 g, 2.342 mmol) in water (3.33 mL) at 0° C. The resulting solution was stirred at rt for 2 h. THF was removed under reduced pressure and the remaining residue was diluted with water (15 mL) and washed with Et₂O (2×10 mL). The aqueous layer was then acidified with 1N HCl to pH ˜2 and extracted with EtOAc (3×15 mL). The combined organic layers were dried (MgSO₄), filtered and concentrated under vacuum to yield Cap-180 (racemic mixture) (0.2 g) as a white foam. LC-MS: Anal. Calcd. for [M+H]⁺ C₁₁H₂₀NO₅: 246.13; found 246.00. ¹H NMR (400 MHz, CDCl₃) δ ppm 5.14 (1H, d, J=9.03 Hz), 4.65 (1H, t, J=9.91 Hz), 3.63-3.89 (5H, m), 1.99-2.13 (1H, m), 1.56-1.73 (2H, m), 1.48-1.55 (1H, m), 1.35-1.48 (1H, m), 1.27 (1H, br. s.), 1.17 (6H, d, J=6.02 Hz).

### Cap-181

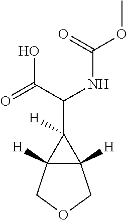

### Cap-181

### Step a

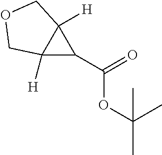

A solution of tert-butyl diazoacetate (1.832 mL, 13.22 mmol) in 50 mL of CH₂Cl₂ was added into a mixture of 2,5-dihydrofuran (9.76 mL, 132 mmol), Rhodium(II) acetate dimer (0.058 g, 0.132 mmol) in 40 mL of CH₂Cl₂ dropwise by a syringe pump over 5 hours. The resulting mixture was then stirred at room temperature overnight. The solvent was removed under vacuum. The residue was purified by chromatography (silica gel, 0%-15% EtOAc/Hex) to afford Cap-181 step a (trans-isomer) (720 mg) and Cap-181, step a (cis-isomer) (360 mg) as clear oil. Cap-181 step a (trans-isomer): ¹H NMR (500 MHz, CDCl₃) δ ppm 3.88 (2H, d, J=8.55 Hz), 3.70 (2H, d, J=8.55 Hz), 2.03-2.07 (2H, m), 1.47 (1H, t, J=3.20 Hz), 1.41 (9H, s); Cap-181, step a (cis-isomer): ¹H NMR (400 MHz, CDCl₃) δ ppm 4.06 (2H, d, J=8.53 Hz), 3.73 (2H, d, J=8.03 Hz), 1.81-1.86 (2H, m), 1.65-1.71 (1H, m), 1.43-1.47 (9H, m).

### Cap-181

### Step b

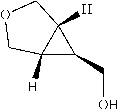

To a solution of (Cap-181, step a (trans-isomer)) (700 mg, 3.80 mmol) in 15 mL of diethyl ether at −10° C. was added LiAlH₄ (7.60 mL, 7.60 mmol) (1 M in THF) dropwise over 1 hour. The resulting mixture was stirred at −10° C. for 1 hour then at room temperature for 1 hour. The mixture was then cooled to −5° C. 10 mL of Rochelle's salt (potassium sodium tartrate) aqueous solution was added dropwise to quench the reaction. The mixture was stirred at room temperature for 30 min and then extracted with EtOAc (3×). The combined organic layers were dried with MgSO₄ and concentrated to afford Cap-181, step b (380 mg) as light yellow oil. The product was used in the next step without purification. ¹H NMR (400 MHz, CDCl₃) δ ppm 3.85 (2H, d, J=8.28 Hz), 3.68 (2H, d, J=8.53 Hz), 3.45-3.55 (2H, m), 1.50-1.56 (2H, m), 1.02-1.11 (1H, m).

### Cap-181

### Step c

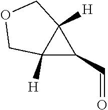

To a solution of DMSO (4.82 mL, 67.9 mmol) in CH₂Cl₂ (70 mL) was added dropwise oxalyl chloride (3.14 mL, 35.8 mmol) at −78° C. The resulting mixture was stirred at −78° C. for 15 min. A solution of Cap-181, step b (3.10 g, 27.2 mmol) in 35 mL of CH₂Cl₂ was added and the mixture was stirred at −78° C. for 1 hour. Et₃N (18.93 mL, 136 mmol) was then added dropwise. After 30 min, the cooling bath was removed and the reaction was quenched with cold 20% K₂HPO₄ aq. solution (10 mL) and water. The mixture was stirred at room temperature for 15 min and then diluted with Et₂O. The layers were separated. The aqueous layer was extracted with Et₂O (2×). The combined organic layers were washed with brine, dried with MgSO₄ and concentrated. The residue was purified by flash chromatography (silica gel, 100% CH₂Cl₂) to afford Cap-181, step c (2.71 g) as light yellow oil. ¹H NMR (500 MHz, CDCl₃) δ ppm 9.41 (1H, d, J=4.27 Hz), 3.96 (2H, d, J=8.85 Hz), 3.80 (2H, d, J=8.55 Hz), 2.27-2.33 (2H, m), 1.93 (1H, m).

### Cap-181

### Step d

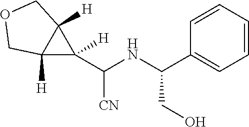

To a mixture of Cap-181, step c (2.7 g, 24.08 mmol) in 50 mL of water at 0° C. was added sodium bisulfate (2.506 g, 24.08 mmol) and KCN (1.631 g, 25.04 mmol), followed by a solution of (R)-2-amino-2-phenylethanol (3.30 g, 24.08 mmol) in 18 mL of MeOH. The resulting mixture was stirred at room temperature for 2 hours and then heated to reflux overnight. The mixture was cooled to room temperature. 100 mL of EtOAc was added. After mixing for 15 min, the layers were separated. The aqueous layer was extracted with EtOAc (2×). The combined organic layers were washed with brine, dried with MgSO₄ and concentrated. The crude diastereomeric mixture was purified by reverse phase HPLC (Column: Water Sunfire 30×150 mm, acetonitrile/water/NH₄OAc) to afford a two diastereomers of Cap-181, step d. The absolute stereochemistry of each isomer was not determined Diastereomer 1 (later eluting fraction) (570 mg): LC-MS: Anal. Calcd. for [M+H]⁺ C₁₅H₁₉N₂O₂ 259.14; found 259.2.

### Cap-181

### Step e

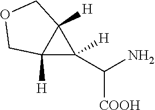

To a solution of Cap-181, step d (diastereomer 1) (570 mg, 2.207 mmol) in 20 mL of CH₂Cl₂ and 20 mL of MeOH at 0° C. was added lead tetraacetate (1174 mg, 2.65 mmol). The resulting orange mixture was stirred at 0° C. for 10 min. Water (20 mL) was then added into the mixture and the mixture was filtered off (CELITE®). The filtrate was concentrated and diluted with 25 mL of 6 N HCl aq. solution. The resulting mixture was refluxed for 4 hours. The mixture was filtered off and washed with CH₂Cl₂. The aqueous layer was concentrated to afford Cap-181, step e (HCl salt). The crude product was used in the next step without further purification. ¹H NMR (500 MHz, MeOD) δ ppm 3.87-3.91 (2H, m), 3.73 (2H, dd, J=8.70, 2.90 Hz), 3.55 (1H, d, J=10.07 Hz), 2.02-2.07 (1H, m), 1.94-1.99 (1H, m), 1.03-1.10 (1H, m).

### Cap-181

To a mixture of the above crude Cap-181, step e in 1 N NaOH aq. solution (10 mL) was added sodium bicarbonate (371 mg, 4.42 mmol). Methyl chloroformate (0.342 mL, 4.42 mmol) was then added dropwise, and the resulting mixture was stirred at room temperature for 3 hours. The mixture was neutralized with 1 N HCl aq. solution and extracted with EtOAc (3×). The combined organic layers were dried with MgSO₄ and concentrated to afford Cap-181 (100 mg, 21% over two steps) as light yellow oil. LC-MS: Anal. Calcd. for [M+H]⁺ C₉H₁₄NO₅ 216.09; found 216.1. ¹H NMR (500 MHz, CDCl₃) δ ppm 5.29 (1H, br. s.), 3.53-4.02 (8H, m), 1.66-1.92 (2H, m), 1.08 (1H, br. s.).

### Cap-182

### Racemic Mixture

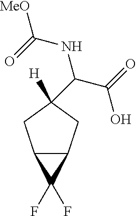

### Cap-182 Step a

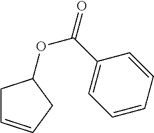

A solution of cyclopent-3-enol (5 g, 59.4 mmol) and Et₃N (9.94 mL, 71.3 mmol) in 50 mL of CH₂Cl₂ was stirred at room temperature for 15 min. Benzoyl chloride (8.28 mL, 71.3 mmol) was then added dropwise and the mixture was stirred at room temperature overnight. The mixture was then washed with water, and the organic layer was dried with MgSO₄ and concentrated. The residue was purified by flash chromatography (silica gel, EtOAc/Hex 0-10%) to afford Cap-182, step a (9.25 g) as clear oil. ¹H NMR (400 MHz, CDCl₃) δ ppm 8.01-8.07 (2H, m), 7.55 (1H, t, J=7.40 Hz), 7.43 (2H, t, J=7.65 Hz), 5.79 (2H, s), 5.64 (1H, tt, J=6.93, 2.60 Hz), 2.87 (2H, dd, J=16.56, 6.78 Hz), 2.52-2.63 (2H, m).

### Cap-182

### Step b

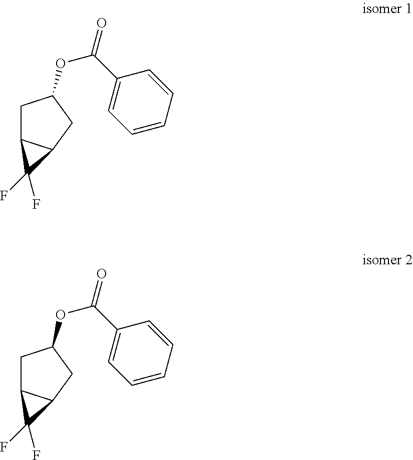

To a round bottom flask with a magnetic stirring bar was added sodium fluoride (5.02 mg, 0.120 mmol) and Cap-182, step a (2.25 g, 11.95 mmol). The flask was heated up to 100° C. and neat trimethylsilyl 2,2-difluoro-2-(fluorosulfonyl)acetate (5.89 mL, 29.9 mmol) was added slowly by syringe pump over 5 hours, and heated at 100° C. overnight. The mixture was then diluted with CH₂Cl₂, washed with water, sat. NaHCO₃ aq. solution and brine, dried with MgSO₄ and concentrated. The crude product was purified by flash chromatography (silica gel, 0-5% EtOAc/Hex) to afford Cap-182, step b (isomer 1) (750 mg) and Cap-182, step b (isomer 2) (480 mg) as clear oils. Relative stereochemical assignment was made by NOE study. Cap-182, step b (isomer 1): LC-MS: Anal. Calcd. for [M+H]⁺ C₁₃H₁₃F₂O₂ 239.09; found 239.2. ¹H NMR (500 MHz, CDCl₃) δ ppm 7.99-8.04 (2H, m), 7.56 (1H, t, J=7.32 Hz), 7.43 (2H, t, J=7.63 Hz), 5.25-5.33 (1H, m), 2.50 (2 H, dd, J=14.04, 6.71 Hz), 2.14-2.22 (2H, m), 2.08-2.14 (2H, m). Cap-182, step b (isomer 2): LC-MS: Anal. Calcd. for [M+H]⁺ C₁₃H₁₃F₂O₂ 239.09; found 239.2. ¹H NMR (400 MHz, CDCl₃) δ ppm 7.98-8.08 (2H, m), 7.53-7.59 (1H, m), 7.41-7.48 (2H, m), 5.53-5.62 (1H, m), 2.59-2.70 (2H, m), 2.01-2.11 (4H, m).

### Cap-182

### Step c

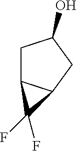

To a solution of Cap-182, step b (isomer 2) (480 mg, 2.015 mmol) in 4 mL of MeOH was added KOH (4 mL, 2.015 mmol) (10% aq.). The resulting mixture was stirred at room temperature overnight. The mixture was then extracted with CH₂Cl₂ (3×). The combined organic layers were dried with MgSO₄ and concentrated to afford Cap-182, step c (220 mg) as a light yellow solid. ¹H NMR (500 MHz, CDCl₃) δ ppm 4.41-4.54 (1H, m), 2.38-2.50 (2H, m), 1.89-1.99 (2H, m), 1.81 (2H, dd, J=14.50, 5.04 Hz).

### Cap-182

### Step d

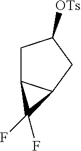

p-Tosyl-Cl (625 mg, 3.28 mmol) was added to a solution of Cap-182, step c (220 mg, 1.640 mmol) and pyridine (0.531 mL, 6.56 mmol) in 7 mL of CH₂Cl₂. The mixture was stirred at room temperature overnight and then diluted with CH₂Cl₂, washed with water and 1 N HCl aq. solution. The organic layer was dried (MgSO₄) and concentrated. The residue was purified by flash chromatography (silica gel, 0-15% EtOAc/Hexane) to afford Cap-182, step d (325 mg) as a clear oil. LC-MS: Anal. Calcd. For [M+Na]⁺ C₁₃H₁₄F₂NaO₃S 311.05; found 311.2. ¹H NMR (500 MHz, CDCl₃) δ ppm 7.76 (2H, d, J=8.24 Hz), 7.34 (2H, d, J=8.24 Hz), 4.99-5.08 (1H, m), 2.45 (3H, s), 2.31-2.41 (2H, m), 1.84-1.94 (4H, m).

### Cap-182

### Step e (Racemic Mixture)

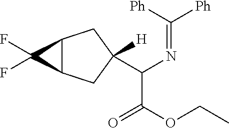

To a microwave tube was added N-(diphenylmethylene)glycine ethyl ester (241 mg, 0.902 mmol) and Cap-182, step d (260 mg, 0.902 mmol) in 2 mL of toluene. The tube was sealed and LiHMDS (1.082 mL of 1 N in THF, 1.082 mmol) was added dropwise under N₂. The resulting dark brown solution was heated at 100° C. in microwave for 5 hours. The mixture was then quenched with water, and extracted with EtOAc (3×). The combined organic layers were washed with water, dried with MgSO₄ and concentrated. The crude product was purified by flash chromatography (silica gel, 0-5% EtOAc/Hex) to afford a racemic mixture of Cap-182, step e (240 mg) as light yellow oil. The mixture was submitted to the next step without separation. LC-MS: Anal. Calcd. for [M+H]⁺ C₂₃H₂₄F₂NO₂ 384.18, found 384.35. ¹H NMR (500 MHz, CDCl₃) δ ppm 7.63-7.70 (2H, m), 7.43-7.51 (3H, m), 7.38-7.43 (1H, m), 7.31-7.38 (2H, m), 7.13-7.22 (2H, m), 4.13-4.22 (2H, m), 3.95 (1H, d, J=6.41 Hz), 2.67-2.79 (1H, m), 2.07-2.16 (1H, m), 1.97-2.07 (2 H, m), 1.90 (2H, m), 1.65-1.76 (1H, m), 1.25 (3H, t, J=7.17 Hz).

### Cap-182

### Step f (Racemic Mixture)

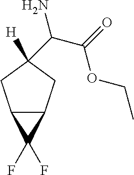

To a solution of Cap-182, step e (240 mg, 0.626 mmol) in 4 mL of THF was added HCl (1 mL, 2.0 mmol) (2 N aq.). The resulting mixture was stirred at room temperature for 2 hours. The mixture was then washed with EtOAc, neutralized with sat. NaHCO₃ aq. solution and then extracted with EtOAc (3×). The combined organic layers were dried with MgSO₄ and concentrated to afford Cap-182, step f (120 mg) as clear oil. LC-MS: Anal. Calcd. for [M+H]⁺ C₁₀H₁₆F₂NO₂ 220.11; found 220.26. ¹H NMR (500 MHz, CDCl₃) δ ppm 4.14-4.25 (2H, m), 3.26 (1H, d, J=6.71 Hz), 2.22-2.35 (1H, m), 1.90-2.11 (5H, m), 1.79-1.90 (1H, m), 1.22-1.34 (3H, m).

### Cap-182

### Step g (Racemic Mixture)

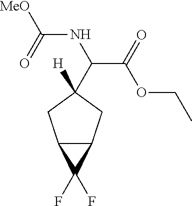

To a solution of Cap-182, step f (120 mg, 0.547 mmol) in 2 mL of CH₂Cl₂ was added methyl chloroformate (0.085 mL, 1.095 mmol). The resulting mixture was stirred at room temperature for 1 hour. The mixture was diluted with CH₂Cl₂ and washed with water. The organic layer was dried with Na₂SO₄ and concentrated to afford Cap-182, step g (150 mg) as a white solid. LC-MS: Anal. Calcd. for [M+H]⁺ C₁₂H₁₈F₂NO₄ 278.12; found 278.2. ¹H NMR (500 MHz, CDCl₃) δ ppm 5.23 (1H, d, J=8.24 Hz), 4.29 (1H, t, J=7.48 Hz), 4.15-4.23 (2H, m), 3.68 (3H, s), 2.37 (1H, br. s.), 2.02-2.10 (1H, m), 1.85-2.00 (4H, m), 1.75-1.84 (1H, m), 1.27 (3 H, t, J=7.02 Hz).

### Cap-182

### Racemic Mixture

To a mixture of Cap-182, step g (150 mg, 0.541 mmol) in 2 mL of THF and 1 mL of water was added LiOH (0.811 mL, 1.623 mmol) (2 N aq.). The resulting mixture was stirred at room temperature overnight. The mixture was neutralized with 1 N HCl aq. solution and extracted with EtOAc (3×). The combined organic layers were dried with MgSO₄ and concentrated to afford Cap-182 (133 mg) as a white solid. LC-MS: Anal. Calcd. for [M+H]⁺ C₁₀H₁₄F₂NO₄ 250.09; found 250.13. ¹H NMR (500 MHz, CDCl₃) δ ppm 5.18-5.36 (1H, m), 4.28-4.44 (1H, m), 3.70 (3H, s), 2.37-2.56 (1H, m), 1.74-2.31 (6H, m).

### Cap-183

### Racemic Mixture

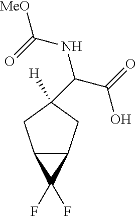

Cap-183 was synthesized from Cap-182, step b (isomer 1) according to the procedure described for the preparation of Cap-182. Anal. Calcd. for [M+H]⁺ C₁₀H₁₄F₂NO₄ 250.09, found 249.86. ¹H NMR (500 MHz, CDCl₃) δ ppm 5.15 (1H, d, J=8.24 Hz), 4.32 (1H, t, J=7.48 Hz), 3.69 (3H, s), 2.83-2.99 (1H, m), 1.96-2.26 (4H, m), 1.70 (1H, t, J=11.75 Hz), 1.59 (1H, t, J=12.05 Hz).

### Cap-184

### Racemic Mixture

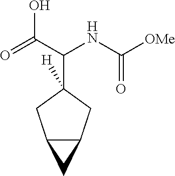

### Cap-184

### Step a

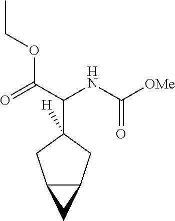

A mixture of ethyl 2-amino-2-((1R,3r,5S)-bicyclo[3.1.0]hexan-3-yl)acetate (prepared from commercially available (1R,3r,5S)-bicyclo[3.1.0]hexan-3-ol by employing the same procedures described for the preparation of Cap-182; 350 mg, 1.910 mmol), DiPEA (0.667 mL, 3.82 mmol), methyl chloroformate (0.296 mL, 3.82 mmol) in 5 mL of CH₂Cl₂ was stirred at room temperature for 1 hour. The mixture was then diluted with CH₂Cl₂ and washed with water. The organic layer was dried with MgSO₄ and concentrated to afford Cap-184, step a (461 mg) as yellow oil. LC-MS: Anal. Calcd. for [M+H]⁺ C₁₂H₂₀NO₄ 242.14; found 242.2. ¹H NMR (500 MHz, CDCl₃) δ ppm 5.04 (1H, d, J=7.63 Hz), 4.09-4.20 (2H, m), 4.05 (1H, t, J=8.39 Hz), 3.63 (3H, s), 2.55-2.70 (1H, m), 1.96-2.09 (2H, m), 1.37-1.60 (4H, m), 1.24 (3H, t, J=7.17 Hz), 0.66-0.76 (1H, m), −0.03-0.06 (1H, m).

### Cap-184

### Racemic Mixture

To a mixture of Cap-184, step a (461 mg, 1.911 mmol) in 5 mL of THF and 2 mL of water was added LiOH (2.87 mL, 5.73 mmol) (2 N aq.). The resulting mixture was stirred at room temperature overnight. The mixture was then neutralized with 1 N HCl aqueous solution, and extracted with EtOAc (3×). The combined organic layers were dried with MgSO₄ and concentrated to afford Cap-184 (350 mg) as clear oil. LC-MS: Anal. Calcd. for [2M+Na]⁺ C₂₀H₃₀N₂NaO₈ 449.19; found 449.3. ¹H NMR (500 MHz, CDCl₃) δ ppm 5.07 (1H, d, J=8.85 Hz), 4.13 (1H, t, J=8.24 Hz), 3.68 (3H, s), 2.64-2.79 (1H, m), 2.04-2.21 (2H, m), 1.23-1.49 (4H, m), 0.71-0.81 (1H, m), 0.03-0.12 (1H, m).

### Cap-185

### Enantiomer-1 and Enantiomer-2

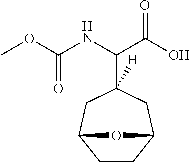

### Cap-185

### Step a

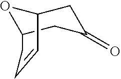

To a mixture of furan (1.075 mL, 14.69 mmol) and zinc (1.585 g, 24.24 mmol) in 1 mL of THF was added 1,1,3,3-tetrabromopropan-2-one (8.23 g, 22.03 mmol) and triethyl borate (5.25 mL, 30.8 mmol) in 4 mL of THF dropwise during 1 hour in dark. The resulting mixture was stirred at room temperature in dark for 17 hours. The resulting dark brown mixture was cooled to −15° C., and 6 mL of water was added. The mixture was warmed to 0° C. and stirred at this temperature for 30 min. The mixture was then filtered and washed with ether. The filtrate was diluted with water and extracted with ether (3×). The combined organic layers were dried with MgSO₄ and concentrated to afford dark brown oil. The dark brown oil was dissolved in 6 mL of MeOH and the solution was added dropwise to a mixture of zinc (4.99 g, 76 mmol), copper (I) chloride (0.756 g, 7.64 mmol) and ammonium chloride (5.4 g, 101 mmol) in 20 mL of MeOH. The reaction temperature was maintained below 15° C. during addition. The mixture was then stirred at room temperature for 20 hours, filtered, and the filtrate was diluted with water and extracted with CH₂Cl₂ (3×). The combined organic layers were dried with MgSO₄ and concentrated. The crude product was purified by flash chromatography (silica gel, 0-14% EtOAc/Hex) to afford Cap-185, step a as a white solid (1.0 g) as a white solid, which turned yellow soon. ¹H NMR (500 MHz, CDCl₃) δ ppm 6.24 (2H, s), 5.01 (2H, d, J=4.88 Hz), 2.73 (2H, dd, J=16.94, 5.04 Hz), 2.31 (2H, d, J=16.79 Hz).

### Cap-185

### Step b

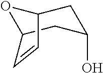

To a solution of Cap-185, step a (240 mg, 1.933 mmol) in 2 mL of THF at −78° C. was added L-selectride (3.87 mL, 3.87 mmol) (1 M in THF) dropwise over 100 min. The resulting mixture was stirred at −78° C. for 1 hour and then at room temperature overnight. The mixture was then cooled to 0° C., 4 mL of 20% NaOH aqueous solution was added, followed by 2 mL of H₂O₂ (30% water solution) dropwise. The resulting mixture was stirred for 1 hour and then neutralized with 6N HCl (˜5 mL). The aqueous layer was saturated with NaCl and extracted with CH₂Cl₂ (3×). The combined organic layers were dried with MgSO₄ and concentrated. The crude product was purified by flash chromatography (silica gel, 0-40% EtOAc/Hex) to afford Cap-185, step b (180 mg) as clear oil. ¹H NMR (400 MHz, CDCl₃) δ ppm 6.49 (2H, s), 4.76 (2H, d, J=4.27 Hz), 3.99 (1H, t, J=5.77 Hz), 2.29 (2H, ddd, J=15.18, 5.65, 4.02 Hz), 1.70-1.78 (2H, m).

### Cap-185

### Step c

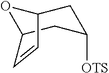

p-Tosyl-Cl (544 mg, 2.85 mmol) was added to a solution of Cap-185, step b (180 mg, 1.427 mmol) and pyridine (0.462 mL, 5.71 mmol) in 5 mL of CH₂Cl₂ (5 mL) and the mixture was stirred at room temperature for 2 days. The reaction was diluted with CH₂Cl₂ and washed with 1 N aq. HCl. The aqueous layer was extracted with CH₂Cl₂ (2×). The combined organic layers were dried with MgSO₄ and concentrated. The crude product was purified by flash chromatography (silica gel, 0-15% EtOAc/Hex) to afford Cap-185, step c (210 mg) as a white solid. ¹H NMR (500 MHz, CDCl₃) δ ppm 7.73 (2H, d, J=8.24 Hz), 7.32 (2H, d, J=8.24 Hz), 6.25 (2H, s), 4.76 (1H, t, J=5.65 Hz), 4.64 (2H, d, J=3.66 Hz), 2.44 (3H, s), 2.18 (2H, td, J=10.07, 5.49 Hz), 1.71 (2H, d, J=15.56 Hz).

### Cap-185

### Step d

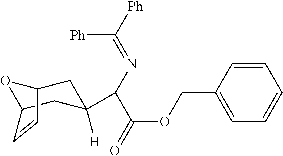

A microwave tube was charged with benzyl 2-(diphenylmethyleneamino)acetate (1.5 g, 4.57 mmol) and Cap-185, step c (1.28 g, 4.57 mmol) in 5 mL of toluene. The tube was sealed and LiHMDS (5.5 mL, 5.5 mmol) (1 N in toluene) was added dropwise under N₂. The resulting dark brown solution was heated at 100° C. in microwave for 5 hours. To the mixture was then added water and EtOAc. The layers were separated and the water phase was extracted with EtOAc (2×). The combined organic layers were concentrated to afford Cap-185, step d as a racemic mixture of The crude mixture was submitted to the next step without purification or separation. LC-MS: Anal. Calcd. for [M+H]⁺ C₂₉H₂₈NO₃ 438.21; found 438.4.

### Cap-185

### Step e

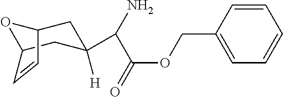

To a solution of the racemic mixture of Cap-185, step d in 30 mL of THF was added HCl (20 mL) (2 N aq.). The resulting mixture was stirred at room temperature for 2 hours. After the reaction was done as judged by TLC, the two layers were separated. The aqueous layer was washed with EtOAc, neutralized with sat. NaHCO₃ aq. solution and then extracted with EtOAc (3×). The combined organic layers were dried with MgSO₄ and concentrated to afford Cap-185, step e. LC-MS: Anal. Calcd. for [M+H]⁺ C₁₆H₂₀NO₃ 274.14; found 274.12.

### Cap-185

### Step f

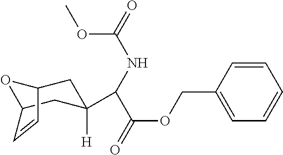

A solution of the crude Cap-185, step e, DiPEA (1.24 mL, 7.1 mmol) and methyl chloroformate (0.55 mL, 7.1 mmol) in 5 mL of CH₂Cl₂ was stirred at room temperature for 1 hour. The mixture was then diluted with CH₂Cl₂ and washed with water. The organic layer was dried with Na₂SO₄ and concentrated. The crude product was purified by flash chromatography (silica gel, 0-40% EtOAc/Hex) to afford 700 mg of the racemic mixture. The mixture was then separated by chiral HPLC (CHIRALPAK® AD-H column, 30×250 mm, 5 um) eluting with 88% CO₂-12% EtOH at 70 mL/min to afford 240 mg of Enantiomer-1 and 310 mg of Enantiomer-2 of Cap-1, step f as white solids. Enantiomer-1: LC-MS: Anal. Calcd. for [M+H]⁺ C₁₈H₂₂NO₅ 332.15; found 332.3. ¹H NMR (500 MHz, CDCl₃) δ ppm 7.30-7.40 (5H, m), 6.03-6.16 (2H, m), 5.09-5.26 (3H, m), 4.65-4.74 (2H, m), 4.33 (1H, dd, J=9.16, 4.88 Hz), 3.67 (3H, s), 2.27-2.38 (1H, m), 1.61-1.69 (1H, m), 1.45-1.56 (1H, m), 1.34 (1H, dd, J=13.43, 5.19 Hz), 1.07 (1H, dd, J=13.12, 5.19 Hz). Enantiomer-2: LC-MS: Anal. Calcd. for [M+H]⁺ C₁₈H₂₂NO₅ 332.15; found 332.06.

### Cap-185

### Enantiomer-1 and Enantiomer-2

To a hydrogenation bottle containing a solution Cap-185, step f (Enantiomer-2) (300 mg, 0.905 mmol) in 10 mL of MeOH was added Pd/C (15 mg, 0.141 mmol) under a cover of nitrogen. The mixture was hydrogenated on a Parr shaker at 40 psi for 3 hours. The mixture was then filtered and the filtrate was concentrated to afford Cap-185 (Enantiomer-2) (200 mg) as a white solid. LC-MS: Anal. Calcd. for [M+H]⁺ C₁₁H₁₈NO₅ 244.12; found 244.2. ¹H NMR (500 MHz, CDCl₃) δ ppm 5.33 (1H, br. s.), 4.46 (2H, d), 4.28 (1H, br. s.), 3.68 (3H, s), 2.35 (1H, br. s.), 1.91-2.03 (2H, m), 1.56-1.80 (4H, m), 1.36-1.55 (2H, m). [Note: Cap-185 (Enantiomer-1) can be obtained in a similar fashion.]

### Cap-186

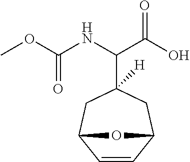

To a solution of the ester Cap-185, step f (Enantiomer-2) (150 mg, 0.453 mmol) in 4 mL of MeOH was added NaOH (4 mL of 1 N in water, 4.00 mmol). The resulting mixture was stirred at room temperature for 3 hours. The methanol was then removed under vacuum, and the residue was neutralized with 1 N HCl solution and extracted with EtOAc (3×). The combined organic layers were dried with MgSO₄ and concentrated to afford Cap-186 that was contaminated with some benzyl alcohol (sticky white solid; 115 mg). LC-MS: Anal. Calcd. for [M+H]⁺ C₁₁H₁₆NO₅ 242.10; found 242.1. ¹H NMR (500 MHz, CDCl₃) δ ppm 6.10-6.19 (2H, m), 5.36 (1H, d, J=8.85 Hz), 4.75-4.84 (2H, m), 4.28 (1H, dd, J=8.55, 4.58 Hz), 3.68 (3H, s), 2.33-2.45 (1H, m), 1.60-1.72 (2H, m), 1.30-1.48 (2H, m).

### Cap-187

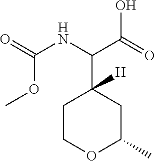

### Cap-187

### Step a

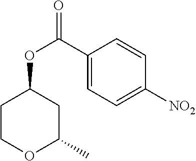

To a solution of Cap-178, step e (2.2 g, 18.94 mmol), PPh₃ (24.84 g, 95 mmol) and 4-nitrobenzoic acid (14.24 g, 85 mmol) in 30 mL of benzene was added DEAD (42.9 mL, 95 mmol) dropwise. The resulting light orange solution was stirred at room temperature overnight. The solvent was then removed under vacuum and the residue was purified by flash chromatography (silica gel, 0-15% EtOAc/Hex) to afford Cap-187, step a (2.3 g) as a white solid. ¹H NMR (500 MHz, CDCl₃) δ ppm 8.27-8.34 (2H, m), 8.20-8.26 (2H, m), 5.45 (1H, t, J=2.90 Hz), 3.83-3.96 (3H, m), 1.90-2.03 (2H, m), 1.80-1.88 (1H, m), 1.61-1.70 (1H, m), 1.21 (3H, d, J=6.10 Hz).

### Cap-187

### Step b

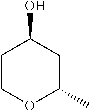

To a solution of Cap-187, step a (2.3 g, 8.67 mmol) in 10 mL of MeOH was added sodium methoxide (2.372 mL, 8.67 mmol) (25% in Methanol). The resulting mixture was stirred at room temperature for 3 hours. Water was added, and the mixture was extracted with EtOAc (5×). The combined organic layers were dried with MgSO₄ and concentrated. The crude product was purified by flash chromatography (silica gel, 0-15% EtOAc/Hex, then 15-50% EtOAc/Hex) to afford Cap-187, step b (0.85 g) as clear oil. ¹H NMR (500 MHz, CDCl₃) δ ppm 4.19-4.23 (1H, m), 3.82-3.91 (2H, m), 3.73-3.79 (1H, m), 1.79-1.88 (1H, m), 1.62-1.68 (1H, m), 1.46-1.58 (2H, m), 1.14 (3H, d, J=6.10 Hz).

### Cap-187

The individual diastereomers of Cap-187 were synthesized from Cap-187, step b according to the procedure described for Cap-178. LC-MS: Anal. Calcd. for [M+H]⁺ C₁₀H₁₈NO₅ 232.12; found 232.1. ¹H NMR (400 MHz, CDCl₃) δ ppm 5.26 (1H, d, J=7.78 Hz), 4.32-4.43 (1H, m), 4.07 (1H, dd, J=11.54, 3.51 Hz), 3.72 (3 H, s), 3.39-3.50 (2H, m), 2.08-2.23 (1H, m), 1.54-1.68 (1H, m), 1.38-1.52 (1 H, m), 1.11-1.32 (5H, m).

### Cap-188

### Four Stereoisomers

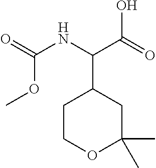

### Cap-188

### Step a

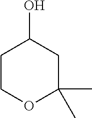

To a solution of 2,2-dimethyldihydro-2H-pyran-4(3H)-one (2 g, 15.60 mmol) in 50 mL of MeOH was slowly added sodium borohydride (0.649 g, 17.16 mmol). The resulting mixture was stirred at room temperature for 3 hours. To the mixture was then added 1 N HCl aqueous solution until it crosses into acidic pH range and then extracted with EtOAc (3×). The combined organic layers were dried with MgSO₄ and concentrated to afford Cap-188, step a (1.9 g) as clear oil. The product was used in the next step without purification. ¹H NMR (400 MHz, CDCl₃) δ ppm 3.91-4.02 (1H, m), 3.79-3.86 (1H, m), 3.63 (1H, td, J=12.05, 2.51 Hz), 1.82-1.93 (2H, m), 1.40-1.53 (1H, m), 1.29-1.38 (1H, m), 1.27 (3H, s), 1.20 (3H, s).

### Cap-188.1 and Cap-188.2

### Step b

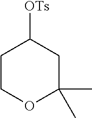

p-Tosyl-Cl (5.56 g, 29.2 mmol) was added to a solution of Cap-188, step a (1.9 g, 14.59 mmol) and pyridine (4.72 mL, 58.4 mmol) in 100 mL of CH₂Cl₂. The resulting mixture was stirred at room temperature for 3 days. To the reaction was added 10 mL of water, and the mixture was stirred at room temperature for an additional hour. The two layers were separated and the organic phase was washed with water and 1 N HCl aqueous solution. The organic phase was dried with MgSO₄ and concentrated to afford the mixture of two enantiomers as a light yellow solid. The mixture was then separated by chiral HPLC (CHIRALPAK® AD column, 21×250 mm, 10 um) eluting with 92% 0.1% diethylamine/Heptane-8% EtOH at 15 mL/min to afford Cap-188.1, step b (1.0 g) and Cap-188.2, step b (1.0 g). The absolute stereochemistry of the two enantiomers was not assigned. Cap-188.1, step b: LC-MS: Anal. Calcd. for [2M+Na]⁺ C₂₈H₄₀NaO₈S₂ 591.21; found 591.3. ¹H NMR (500 MHz, CDCl₃) δ ppm 7.79 (2H, d, J=8.24 Hz), 7.34 (2H, d, J=8.24 Hz), 4.72-4.81 (1H, m), 3.78 (1H, dt, J=12.44, 4.16 Hz), 3.53-3.61 (1H, m), 2.45 (3H, s), 1.75-1.86 (2H, m), 1.61-1.71 (1H, m), 1.52-1.60 (1H, m), 1.22 (3H, s), 1.14 (3 H, s). Cap-188.2, step b: LC-MS: Anal. Calcd. for [2M+Na]⁺ C₂₈H₄₀NaO₈S₂ 591.21; found 591.3.

### Cap-188

The four stereoisomers of Cap-188 could be synthesized from Cap-188.1, step b and Cap-188.2, step b, according to the procedure described for the preparation of Cap-178. Cap-188 (Steroisomer-1): LC-MS: Anal. Calcd. for [M+Na]⁺ C₁₁H₁₉NNaO₅ 268.12; found 268.23. ¹H NMR (500 MHz, CDCl₃) δ ppm 5.32 (1H, d, J=8.55 Hz), 4.26-4.35 (1H, m), 3.57-3.82 (5H, m), 2.11-2.34 (1H, m), 1.25-1.58 (4H, m), 1.21 (6H, d, J=6.10 Hz). Cap-188 (Stereoisomer-2): LC-MS: Anal. Calcd. for [M+H]⁺ C₁₁H₂₀NO₅ 246.13; found 246.1. ¹H NMR (500 MHz, CDCl₃) δ ppm 5.25 (1H, d, J=8.55 Hz), 4.33 (1H, dd, J=8.39, 5.04 Hz), 3.80 (1H, dd, J=11.90, 3.97 Hz), 3.62-3.76 (4H, m), 2.20-2.32 (1H, m), 1.52-1.63 (1H, m), 1.27-1.49 (3H, m), 1.22 (6H, d, J=14.04 Hz).

### Cap-189

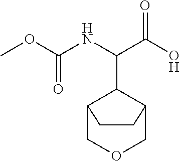

### Cap-189

### Step a

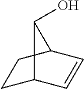

To a solution of phenylmagnesium bromide (113 mL, 340 mmol) (3 M in ether) in 100 mL of ether was added dropwise exo-2,3-epoxynorbornane (25 g, 227 mmol) in 50 mL of ether. After the initial exotherm, the mixture was heated to reflux overnight. The reaction was then cooled to room temperature and quenched carefully with water (˜10 mL). The mixture was diluted with ether and washed with a 3 N HCl aqueous solution (˜160 mL). The aqueous layer was extracted with ether (2×) and the combined organic layers were dried with MgSO₄ and concentrated. The crude product was purified by flash chromatography (silica gel, 0-18% EtOAc/Hex) to afford Cap-189, step a (11 g). ¹H NMR (400 MHz, CDCl₃) δ ppm 6.03-6.11 (2H, m), 3.76 (1H, d, J=11.29 Hz), 2.72-2.81 (2H, m), 1.98 (1H, d, J=11.29 Hz), 1.67-1.76 (2H, m), 0.90-0.97 (2H, m).

### Cap-189

### Step b

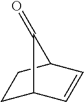

To a solution of oxalyl chloride (59.9 mL, 120 mmol) in 200 mL of CH₂Cl₂ at −78° C. was added DMSO (17.01 mL, 240 mmol) in 100 mL of CH₂Cl₂. The mixture was stirred for 10 min, and Cap-189, step a (11 g, 100 mmol) in 150 mL of CH₂Cl₂ was added followed by Et₃N (72.4 mL, 519 mmol) in 30 mL of CH₂Cl₂. The mixture was stirred at −78° C. for 30 min and then warmed to room temperature. Water (150 mL) was added and the mixture was stirred at room temperature for 30 mins. The two layers were then separated, and the aqueous layer was extracted with CH₂Cl₂ (2×). The organic layers were combined, dried with MgSO₄ and concentrated. The crude product was purified by flash chromatography (silica gel, 0-5% EtOAc/Hex) to afford Cap-189, step b (5.3 g) as a light yellow oil. ¹H NMR (500 MHz, CDCl₃) δ ppm 6.50-6.55 (2H, m), 2.78-2.84 (2H, m), 1.92-1.99 (2H, m), 1.17-1.23 (2H, m).

### Cap-189

### Step c

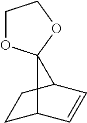

A mixture of Cap-189, step b (5.3 g, 49.0 mmol), p-toluenesulfonic acid monohydrate (1.492 g, 7.84 mmol) and ethylene glycol (4.10 mL, 73.5 mmol) in 100 mL of benzene was refluxed for 4 hours and then stirred at room temperature overnight. The reaction was partitioned between Et₂O and aqueous sat. NaHCO₃ solution and the two layers were separated. The organic layer was washed with brine, dried with MgSO₄ and concentrated. The crude product was purified by flash chromatography (silica gel, 0-6% EtOAc/Hex) to afford Cap-189, step c (5.2 g) as a clear oil. ¹H NMR (400 MHz, CDCl₃) δ ppm 6.20 (2H, t, J=2.13 Hz), 3.90-3.97 (2 H, m), 3.81-3.89 (2H, m), 2.54 (2H, m), 1.89-1.99 (2H, m), 0.95-1.03 (2H, m).

### Cap-189

### Step d

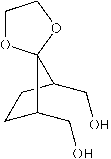

A solution of Cap-189, step c (5.2 g, 34.2 mmol) in 60 mL of MeOH and 50 mL of CH₂Cl₂ was cooled to −78° C. and treated with ozone gas until a light blue color was apparent. The reaction was then bubbled with N₂ to remove the excess ozone gas (blue color disappeared) and sodium borohydride (1.939 g, 51.3 mmol) was added into the reaction. The reaction was then warmed to 0° C. Acetone was added into the mixture to quench the excess sodium borohydride. The mixture was concentrated and the residue was purified by flash chromatography (silica gel, 100% EtOAc) to afford Cap-189, step d (5.0 g) as a clear oil. ¹H NMR (400 MHz, CDCl₃) δ ppm 3.99-4.09 (4H, m), 3.68 (4H, m), 2.17-2.29 (2H, m), 1.92-2.10 (2H, m), 1.77-1.88 (2H, m), 1.57-1.70 (2H, m).

### Cap-189

### Step e

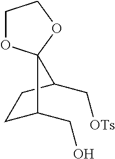

To a solution of Cap-189, step d (1 g, 5.31 mmol) in 20 mL of CH₂Cl₂ was added silver oxide (3.8 g), p-TsCl (1.215 g, 6.38 mmol) and KI (0.176 g, 1.063 mmol). The resulting solution was stirred at room temperature for 3 days. The mixture was then filtered and the filtrate was concentrated. The crude product was purified by flash chromatography (silica gel, 60% EtOAc/Hex) to afford Cap-189, step e (0.79 g) as clear oil. LC-MS: Anal. Calcd. for [M+Na]⁺ C₁₆H₂₂NaO₆S 365.10; found 365.22. ¹H NMR (400 MHz, CDCl₃) δ ppm 7.80 (2H, d, J=8.28 Hz), 7.36 (2 H, d, J=8.03 Hz), 4.11-4.17 (1H, m), 3.85-4.06 (5H, m), 3.64-3.71 (1H, m), 3.55-3.63 (1H, m), 2.47 (3H, s), 2.32-2.43 (1H, m), 2.15-2.27 (1H, m), 1.70-1.89 (2H, m), 1.52-1.66 (1H, m), 1.35-1.47 (1H, m).

### Cap-189

### Step f

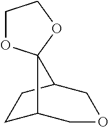

To a solution of Cap-189, step e (2.2 g, 6.43 mmol) in 40 mL of MeOH was added potassium carbonate (1.776 g, 12.85 mmol). The resulting mixture was stirred at room temperature overnight. The mixture was then diluted with water and EtOAc. The two layers were separated. The aqueous layer was extracted with EtOAc (2×). The combined organic layers were washed with brine, dried with MgSO₄ and concentrated. The crude product was purified by flash chromatography (silica gel, 0-15% EtOAc/Hex) to afford Cap-189, step f (0.89 g, 5.23 mmol, 81%) as clear oil. ¹H NMR (400 MHz, CDCl₃) δ ppm 3.89-4.02 (6H, m), 3.58 (2H, dd, J=10.79, 2.51 Hz), 1.69-1.89 (6H, m).

### Cap-189

### Step g

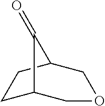

To the solution of Cap-189, step f (890 mg, 5.23 mmol) in 15 mL of THF was added HCl (15 mL, 45.0 mmol) (3 M aqueous). The resulting mixture was stirred at room temperature overnight. The mixture was then diluted with ether and the two layers were separated. The aqueous phase was extracted with ether (2×) and the combined organic layers were dried with MgSO₄ and concentrated to afford Cap-189, step g (0.95 g, containing some residual solvents). The product was used in the next step without purification. ¹H NMR (500 MHz, CDCl₃) δ ppm 3.95-4.00 (2H, m), 3.85 (2H, d, J=10.68 Hz), 2.21-2.28 (2H, m), 1.99-2.04 (2H, m), 1.90-1.96 (2 H, m).

### Cap-189

### Step h (Enantiomer-1 and Enantiomer-2)

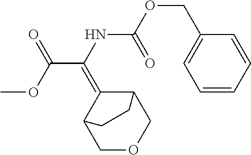

To a solution of (+/−)-benzyloxycarbonyl-a-phosphonoglycine trimethyl ester (1733 mg, 5.23 mmol) in 6 mL of THF at −20° C. was added 1,1,3,3-tetramethylguanidine (0.723 mL, 5.75 mmol). The resultant light yellow mixture was stirred at −20° C. for 1 hour, and Cap-189, step g (660 mg, 5.23 mmol) in 3 mL of THF was added and mixture was then stirred at room temperature for 3 days. The reaction mixture was then diluted with EtOAc, washed with a 0.1 N HCl aq. solution. The aqueous layer was extracted with EtOAc (2×) and the combined organic layers were dried with MgSO₄ and concentrated. The crude product was purified by flash chromatography (silica gel, 0-4% EtOAc/CH₂Cl₂) to afford 960 mg of the racemic mixture. The mixture was separated by chiral HPLC (CHIRALPAK® AD column, 21×250 mm, 10 um) eluting with 90% 0.1% diethylamine/Heptane-10% EtOH at 15 mL/min to afford Cap-189, step h (Enantiomer-1; 300 mg) and Cap-189, step h (Enantiomer-2; 310 mg) as white solids. Cap-189, step h (Enantiomer-1): LC-MS: Anal. Calcd. for [M+H]⁺ C₁₈H₂₂NO₅ 332.15; found 332.2. ¹H NMR (500 MHz, CDCl₃) δ ppm 7.29-7.41 (5H, m), 6.00 (1H, br. s.), 5.13 (2H, s), 3.63-3.87 (8H, m), 2.84 (1H, br. s.), 1.84-2.02 (2H, m), 1.63-1.84 (2H, m). Cap-189, step h (Enantiomer-2): LC-MS: Anal. Calcd. for [M+H]⁺ C₁₈H₂₂NO₅ 332.15; found 332.2.

### Cap-189

### Step i

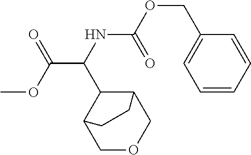

N₂ was bubbled through a solution of Cap-189, step h (Enantiomer-2; 290 mg, 0.875 mmol) in 10 mL of MeOH in a 500 mL hydrogenation bottle for 30 mins To the solution was added (S,S)-Me-BPE-Rh (9.74 mg, 0.018 mmol), and the mixture was then hydrogenated at 60 psi for 6 days. The mixture was concentrated, and chiral analytical HPLC (CHIRALPAK® OJ column) indicated that there were a small amount of remaining starting material and one major product. The residue was then separated by chiral HPLC (CHIRALPAK® OJ column, 21×250 mm, 10 um) eluting with 70% 0.1% diethylamine/Heptane-30% EtOH at 15 mL/min to afford Cap-189, step i, (150 mg) as clear oil. LC-MS: Anal. Calcd. for [M+H]⁺ C₁₈H₂₄NO₅ 334.17; found 334.39. ¹H NMR (500 MHz, CDCl₃) δ ppm 7.28-7.41 (5H, m), 5.12-5.18 (1H, m), 5.09 (2H, s), 4.05 (1H, t, J=10.07 Hz), 3.75 (3H, s), 3.60-3.72 (2 H, m), 3.41-3.50 (2H, m), 2.10 (1H, br. s.), 1.72-1.99 (6H, m).

### Cap-189

### Step j

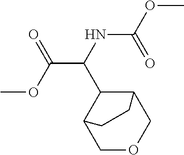

To a solution of Cap-189, step i (150 mg, 0.450 mmol) in 10 mL of MeOH in a hydrogenation bottle were added dimethyl dicarbonate (0.072 mL, 0.675 mmol) and 10% Pd/C (23.94 mg, 0.022 mmol) under a cover of nitrogen cover. The mixture was then hydrogenated on Parr-shaker at 45 psi overnight. The mixture was filtered and the filtrate was concentrated to afford Cap-189, step j (110 mg) as a clear oil. LC-MS: Anal. Calcd. for [M+H]⁺ C₁₂H₂₀NO₅ 258.13; found 258.19. ¹H NMR (500 MHz, CDCl₃) δ ppm 5.08 (1H, d, J=9.16 Hz), 4.03 (1H, t, J=10.07 Hz), 3.75 (3H, s), 3.60-3.72 (5H, m), 3.46 (2H, t, J=10.38 Hz), 2.11 (1H, br. s.), 1.72-1.99 (6H, m).

### Cap-189

To a mixture of Cap-189, step j (110 mg, 0.428 mmol) in 2 mL of THF and 1 mL of water was added LiOH (0.641 mL, 1.283 mmol) (2 N aq.). The resulting mixture was stirred at room temperature overnight. The mixture was neutralized with a 1 N HCl aq. solution and extracted with EtOAc (3×). The combined organic layers were dried with MgSO₄ and concentrated to afford Cap-189 (100 mg) as a white solid. LC-MS: Anal. Calcd. for [M+Na]⁺ C₁₁H₁₇NNaO₅ 266.10; found 266.21. ¹H NMR (500 MHz, CDCl₃) δ ppm 5.10 (1H, d, J=9.16 Hz), 4.02 (1H, t, J=10.07 Hz), 3.62-3.78 (5H, m), 3.49 (2H, d, J=10.68 Hz), 2.07-2.22 (2H, m), 1.72-1.98 (6 H, m).

### Cap-190

### Diastereomeric Mixture

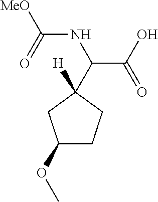

### Cap-190

### Step a

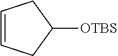

To a mixture of cyclopent-3-enol (2.93 g, 34.8 mmol) and imidazole (5.22 g, 77 mmol) in 30 mL of DMF at 0° C. was added t-butyldimethylchlorosilane (6.30 g, 41.8 mmol). The resulting colorless mixture was stirred at room temperature overnight. Hexanes and water were then added to the mixture and the two layers were separated. The aqueous layer was extracted with EtOAc (2×) and the combined organic layers were washed with brine, dried with MgSO₄ and concentrated. The crude product was purified by flash chromatography (silica gel, 2% EtOAc/Hex) to afford Cap-190, step a (6.3 g) as a clear oil. ¹H NMR (500 MHz, CDCl₃) δ ppm 5.65 (2H, s), 4.49-4.56 (1H, m), 2.56 (2H, dd, J=15.26, 7.02 Hz), 2.27 (2H, dd, J=15.26, 3.36 Hz), 0.88 (9H, s), 0.06 (6H, s).

### Cap-190

### Step b

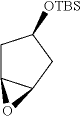

To a solution of Cap-190, step a (2.3 g, 11.59 mmol) in 40 mL of CH₂Cl₂ at 0° C. was added m-CPBA (5.60 g, 16.23 mmol) in 5 portions. The reaction mixture was stirred at room temperature overnight. Hexanes and water were then added to the mixture and the two layers were separated. The organic layer was washed with 50 mL aq. 10% NaHSO₃ and brine, dried with MgSO₄ and concentrated. The crude product was purified by flash chromatography (silica gel, 3%-6% EtOAc/Hex) to afford Cap-190, step b (1.42 g) and its trans diastereomer (0.53 g) as clear oils. Cap-190, step b (cis): ¹H NMR (400 MHz, CDCl₃) δ ppm 4.39-4.47 (1H, m), 3.47 (2H, s), 2.01-2.10 (2H, m), 1.93-2.00 (2H, m), 0.88 (9H, s), 0.04 (6H, s). Cap-190, step b (trans): ¹H NMR (400 MHz, CDCl₃) δ ppm 4.04-4.14 (1H, m), 3.47 (2H, s), 2.41 (2H, dd, J=14.05, 7.28 Hz), 1.61 (2H, dd, J=14.18, 6.90 Hz), 0.87 (9H, s), 0.03 (6H, s).

### Cap-190

### Step c

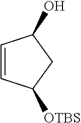

To a solution of (S)-1,2′-methylenedipyrrolidine (0.831 g, 5.39 mmol) in 15 mL of benzene at 0° C. was added dropwise n-butyllithium (4.90 mL, 4.90 mmol) (1 M in hexane). The solution turned bright yellow. The mixture was stirred at 0° C. for 30 min. Cap-190, step b (cis-isomer; 0.7 g, 3.27 mmol) in 10 mL of benzene was then added and the resulting mixture was stirred at 0° C. for 3 hours. EtOAc and sat. NH₄Cl aq. solution were added into the mixture, and the two layers were separated. The organic layer was washed with water and brine, dried with MgSO₄ and concentrated. The crude product was purified by flash chromatography (silica gel, 15% EtOAc/Hex) to afford Cap-190, step c (400 mg) as a light yellow oil. ¹H NMR (500 MHz, CDCl₃) δ ppm 5.84-5.98 (2H, m), 4.53-4.69 (2H, m), 2.63-2.73 (1H, m), 1.51 (1H, dt, J=13.73, 4.43 Hz), 0.89 (9H, s), 0.08 (6H, s).

### Cap-190

### Step d

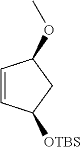

To a solution of Cap-190, step c (400 mg, 1.866 mmol), MeI (1.866 mL, 3.73 mmol) (2 M in t-butyl methyl ether) in 5 mL of THF at 0° C. was added NaH (112 mg, 2.80 mmol) (60% in mineral oil). The resulting mixture was allowed to warm up to room temperature and stirred at room temperature overnight. The reaction was then quenched with water and extracted with EtOAc (3×). The combined organic layers were washed with brine, dried with MgSO₄ and concentrated. The crude product was purified by flash chromatography (silica gel, 5% EtOAc/Hex) to afford Cap-190, step d (370 mg) as light yellow oil. ¹H NMR (500 MHz, CDCl₃) δ ppm 5.92-5.96 (1H, m), 5.87-5.91 (1H, m), 4.64-4.69 (1H, m), 4.23-4.28 (1H, m), 3.32 (3H, s), 2.62-2.69 (1H, m), 1.54 (1H, dt, J=13.12, 5.49 Hz), 0.89 (9H, s), 0.07 (5H, d, J=1.83 Hz).

### Cap-190

### Step e

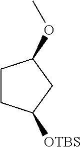

To a solution of Cap-190, step d (400 mg, 1.751 mmol) in 10 mL of EtOAc in a hydrogenation bottle was added platinum(IV) oxide (50 mg, 0.220 mmol). The resulting mixture was hydrogenated at 50 psi on Parr shaker for 2 hours. The mixture was then filtered through CELITE®, and the filtrate was concentrated to afford Cap-190, step e (400 mg) as a clear oil. LC-MS: Anal. Calcd. for [M+H]⁺ C₁₂H₂₇O₂Si 231.18; found 231.3. ¹H NMR (500 MHz, CDCl₃) δ ppm 4.10-4.17 (1H, m), 3.65-3.74 (1H, m), 3.27 (3H, s), 1.43-1.80 (6H, m), 0.90 (9H, s), 0.09 (6H, s).

### Cap-190

### Step f

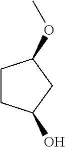

To a solution of Cap-190, step e (400 mg, 1.736 mmol) in 5 mL of THF was added TBAF (3.65 mL, 3.65 mmol) (1 N in THF). The color of the mixture turned brown after several min., and it was stirred at room temperature overnight. The volatile component was removed under vacuum, and the residue was purified by flash chromatography (silica gel, 0-25% EtOAc/Hex) to afford Cap-190, step f (105 mg) as light yellow oil. ¹H NMR (500 MHz, CDCl₃) δ ppm 4.25 (1H, br. s.), 3.84-3.92 (1H, m), 3.29 (3H, s), 1.67-2.02 (6H, m).

### Cap-190

Cap-190 was then synthesized from Cap-190, step f according to the procedure described for Cap-182. LC-MS: Anal. Calcd. for [M+Na]⁺ C₁₀H₁₇NNaO₅ 254.10; found 254.3. ¹H NMR (500 MHz, CDCl₃) δ ppm 5.25 (1H, d, J=8.55 Hz), 4.27-4.41 (1H, m), 3.81-3.90 (1H, m), 3.69 (3H, s), 3.26 (3H, s), 2.46-2.58 (1 H, m), 1.76-1.99 (3H, m), 1.64-1.73 (1H, m), 1.40-1.58 (1H, m), 1.22-1.38 (1H, m).

### Cap-191

### Enantiomer-1

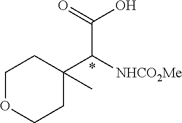

### Cap-191

### Step a

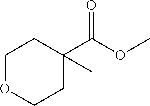

To a solution of diisopropylamine (3 mL, 21.05 mmol) in THF (3 mL) at −78° C. under nitrogen was added n-butyl lithium (2.5 M in hexanes; 8.5 mL, 21.25 mmol). The reaction was stirred at −78° C. for 10 min then brought up to 0° C. for 25 min. The reaction was cooled down again to −78° C., methyl tetrahydro-2H-pyran-4-carboxylate (3 g, 20.81 mmol) in THF (3 mL) was added. The reaction was stirred at −78° C. for 15 min then brought up to 0° C. for 30 min. The reaction was cooled down to −78° C., methyl iodide (1.301 mL, 20.81 mmol) was added. After the addition, the cold bath was removed and the reaction was allowed to slowly warm up to −25° C. and stirred for 22 h. Ethyl acetate and aqueous HCl (0.1N) were added, and the organic layer was separated and washed with brine and dried (MgSO₄), filtered, and concentrated in vacuo. The residue was loaded on a Thomson's silica gel cartridge eluting with 10% ethyl acetate/hexanes to afford a light yellow oil (2.83 g). ¹H NMR (400 MHz, DMSO-d₆) δ ppm 3.73-3.66 (m, 2H), 3.66 (s, 3H), 3.40-3.30 (m, 2H), 1.95-1.93 (dm, 1H), 1.92-1.90 (dm, 1H), 1.43 (ddd, J=13.74, 9.72, 3.89, 2H), 1.18 (s, 3H).

### Cap-191

### Step b

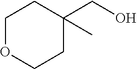

To a solution of Cap-191, step a (3 g, 18.96 mmol) in toluene (190 mL) at −78° C. under nitrogen was added diisobutylaluminum hydride (1.5M in toluene; 26.5 mL, 39.8 mmol) dropwise. The reaction was continued to stir at −78° C. for 1.5 h., and the bath was removed and was stirred for 18 h. The reaction was quenched with MeOH (20 mL). HCl (1M, 150 mL) was added and the mixture was extracted with EtOAc (4×40 mL). The combined organic phases were washed with brine, dried (MgSO₄), filtered, and concentrated in vacuo. The residue was purified with flash chromatography (silica gel; 40% ethyl acetate/hexanes) to afford a colorless oil (1.36 g). ¹H NMR (400 MHz, CDCl₃) δ ppm 3.77 (dt, J=11.73, 4.55, 2H), 3.69-3.60 (m, 2H), 3.42 (s, 2H), 1.71-1.40 (bs, 1H) 1.59 (ddd, J=13.74, 9.72, 4.39, 2H), 1.35-1.31 (m, 1H), 1.31-1.27 (m, 1H), 1.06 (s, 3H).

### Cap-191

### Step c

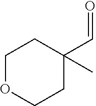

To a solution of DMSO (5.9 mL, 83 mmol) in CH₂Cl₂ (85 mL) at −78° C. under nitrogen was added oxalyl chloride (3.8 mL, 43.4 mmol) and stirred for 40 min. A solution of Cap-191, step b (4.25 g, 32.6 mmol) in CH₂Cl₂ (42.5 mL) was then added. The reaction was continued to be stirred at −78° C. under nitrogen for 2 h. The reaction was quenched with cold 20% K₂HPO₄ (aq) (10 mL) and water. The mixture was stirred at ˜25° C. for 15 min, diluted with diethyl ether (50 mL) and the layers were separated. The aqueous layer was extracted with diethyl ether (2×50 mL). The combined organic layers were washed with brine, dried (MgSO₄), filtered, and concentrated in vacuo. The residue was taken up in CH₂Cl₂ (4 mL) and purified with flash chromatography (silica gel, eluting with CH₂Cl₂) to afford a colorless oil (2.1 g). ¹H NMR (400 MHz, CDCl₃) δ ppm 9.49 (s. 1H), 3.80 (dt, J=11.98, 4.67, 2H), 3.53 (ddd, J=12.05, 9.41, 2.89, 2H), 1.98 (ddd, J=4.71, 3.20, 1.38, 1H), 1.94 (ddd, J=4.71, 3.20, 1.38, 1H), 1.53 (ddd, J=13.87, 9.60, 4.14, 2H), 1.12 (s, 3H).

### Cap-191

### Step d

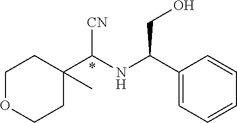

To a solution of Cap 191c (2.5 g, 19.51 mmol) in CHCl₃ (20 mL) under nitrogen at ˜25° C. was added (R)-2-amino-2-phenylethanol (2.94 g, 21.46 mmol) and stirred for 5 h. The reaction was cooled to 0° C., trimethylsilyl cyanide (3.8 mL, 30.4 mmol) was added dropwise. The cold bath was removed and the reaction was allowed to stir at ˜25° C. under nitrogen for 15.5 h. The reaction was treated with 3N

HCl (20 mL) and water (20 mL), and the product was extracted with CHCl₃ (3×50 mL). The combined organic layers were dried (NaSO₄), filtered, and concentrated in vacuo. The residue was purified with flash chromatography (silica gel; 40% ethyl acetate/hexanes) to afford two diastereomers: Cap-191, step d1 (diastereomer 1) as a colorless oil which solidified into a white solid upon standing (3 g). ¹H NMR (400 MHz, DMSO-d₆) δ ppm 7.42-7.26 (m, 5H), 5.21 (t, J=5.77, 1H), 3.87 (dd, J=8.53, 4.52, 1H), 3.61-3.53 (m, 1H), 3.53-3.37 (m, 5H), 3.10 (d, J=13.05, 1H), 2.65 (d, J=13.05, 1H), 1.64-1.55 (m, 1H), 1.55-1.46 (m, 1H), 1.46-1.39 (m, 1H), 1.31-1.23 (m, 1H), 1.11 (s, 3H). LC-MS: Anal. Calcd. for [M+H]⁺ C₁₆H₂₃N₂O₂: 275.18; found 275.20. Cap-191, step d2 (diastereomer 2) as a light yellow oil (0.5 g). ¹H NMR (400 MHz, DMSO-d₆) δ ppm 7.44-7.21 (m, 5H), 4.82 (t, J=5.40, 1H), 3.82-3.73 (m, 1H), 3.73-3.61 (m, 3H), 3.61-3.37 (m, 5H), 2.71 (dd, J=9.29, 4.77, 1H), 1.72-1.55 (m, 2H), 1.48-1.37 (m, 1H), 1.35-1.25 (m, 1H), 1.10 (s, 3H). LC-MS: Anal. Calcd. for [M+H]⁺ C₁₆H₂₃N₂O₂: 275.18; found 275.20.

### Cap-191

### Step e

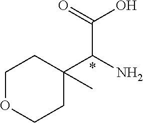

To a solution of Cap-191, step d2 (diastereomer 2) (0.4472 g, 1.630 mmol) in CH₂Cl₂ (11 mL) and MeOH (5.50 mL) at 0° C. under nitrogen was added lead tetraacetate (1.445 g, 3.26 mmol). The reaction was stirred for 1.5 h, the cold bath was removed and stirring was continued for 20 h. The reaction was treated with a phosphate buffer (pH=7; 6 mL) and stirred for 45 min. The reaction was filtered over CELITE®, washed with CH₂Cl₂ and the layers were separated. The aqueous layer was extracted with CH₂Cl₂ (3×25 mL), and the combined organic layers was washed with brine, dried (MgSO₄), filtered and concentrated in vacuo. The residue was purified with flash chromatography (silica gel; 15% ethyl acetate/hexanes) to afford the imine intermediate as a colorless oil (181.2 mg). ¹H NMR (400 MHz, DMSO-d₆) δ ppm 8.55 (d, J=1.00, 1H), 7.89-7.81 (m, 2H), 7.61-7.46 (m, 3H), 4.80 (d, J=1.00, 1H), 3.74 (tt, J=11.80, 4.02, 2H), 3.62-3.46 (m, 2H), 1.79-1.62 (m, 2H), 1.46-1.30 (m, 2H), 1.15 (s, 3H).

The imine intermediate was taken up in 6N HCl (10 mL) and heated at 90° C. for 10 days. The reaction was removed from the heat, allowed to cool to room temperature and extracted with ethyl acetate (3×25 mL). The aqueous layer was concentrated in vacuo to afford an off-white solid. The solid was taken up in MeOH and loaded on a pre-conditioned MCX (6 g) cartridge, washed with MeOH followed by elution with 2N NH₃/MeOH solution and concentrated in vacuo to afford an off-white solid (79.8 mg). ¹H NMR (400 MHz, DMSO-d₆) δ ppm 14.33-13.51 (bs, 1H), 8.30 (bs, 3H), 3.82-3.75 (m, 1H), 3.70 (dt, J=11.80, 4.02, 2H), 3.58-3.43 (m, 2H), 1.76-1.60 (m, 2H), 1.47-1.36 (m, 1H), 1.36-1.27 (m, 1H), 1.08 (s, 3H). LC-MS: Anal. Calcd. for [M+H]⁺ C₈H₁₆NO₃: 174.11; found 174.19.

### Cap-191

### Enantiomer-1

To a solution of Cap-191, step e (0.0669 g, 0.386 mmol) and sodium carbonate (0.020 g, 0.193 mmol) in sodium hydroxide (1M aq.; 0.4 mL, 0.40 mmol) at 0° C. was added methyl chloroformate (0.035 mL, 0.453 mmol) dropwise. The reaction was removed from the cold bath and allowed to stir at ˜25° C. for 3 h. The reaction was washed with diethyl ether (3×20 mL). The aqueous layer was acidified with 12 N HCl (pH ˜1-2), and extracted with ethyl acetate (2×20 mL). The combined organic layers were dried (MgSO₄), filtered, and concentrated in vacuo to afford Cap-191 as a colorless film (66.8 mg). ¹H NMR (400 MHz, DMSO-d₆) δ ppm 13.10-12.37 (bs, 1H), 7.37 (d, J=9.04, 1H), 4.02 (d, J=9.29, 1H), 3.72-3.57 (m, 2H), 3.56 (s, 3H), 3.54-3.44 (m, 2H), 1.65 (ddd, J=13.61, 9.72, 4.27, 1H), 1.53 (ddd, J=13.68, 9.66, 4.27, 1H), 1.41-1.31 (m, 1H), 1.31-1.22 (m, 1H), 1.00 (s, 3H). LC-MS: Anal. Calcd. for [M+Na]⁺ C₁₀H₁₇NO₅Na: 254.10; found 254.11.

### Cap-192

### Enantiomer-2

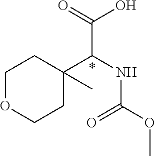

Cap-192 (Enantiomer-2) was prepared from Cap-191, step d1 according to the procedure described for the preparation of its enantiomer Cap-191.

### Cap-193

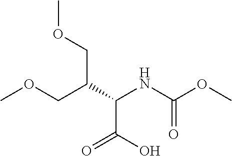

### Cap-193

### Step a

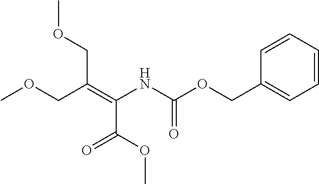

To a solution of methyl 2-(benzyloxycarbonylamino)-2-(dimethoxyphosphoryl)acetate (1.45 g, 4.2 mmol) in DCM was added DBU (0.70 mL, 4.7 mmol). The reaction mixture was stirred for 10 min, followed by addition of a solution of 1,3-dimethoxypropan-2-one (0.5 g, 4.2 mmol) in DCM. The reaction mixture was stirred at room temperature for 18 hrs. The reaction mixture was charged to an 80 g silica gel cartridge which was eluted with an 18 min gradient of 0-70% EtOAc in hexane to afford Cap-193, Step a (0.8 g) as a thick oil. ¹H NMR (400 MHz, MeOD) ppm 7.23-7.43 (5H, m), 4.99-5.18 (2H, m), 4.16 (2H, s), 4.06 (2H, s), 3.66-3.78 (3H, s), 3.26 (3H, s), 3.23 (3H, s). LC-MS: Anal. Calcd. For [M+Na]⁺ C₁₆H₂₁NNaO₆: 346.14; found: 346.12.

### Cap-193

### Step b

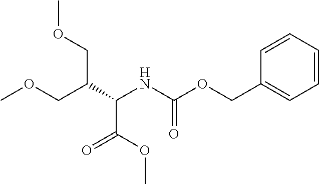

A reaction mixture of ester Cap-193, Step a (0.5 g) and (+)-1,2-bis((2S,5S)-2,5-diethylphospholano)benzene(cyclooctadiene)rhodium (I) tetrafluoroborate (0.1 g) in MeOH was stirred under 55 psi of H₂ for 18 hrs. The reaction mixture was concentrated to dryness. The residue was charged to a 25 g silica gel cartridge and eluted with an 18 min gradient of 0-80% EtOAc in hexane to afford Cap-193, Step b (0.49 g) as a clear oil. LC-MS: Anal. Calcd. For [M+Na]⁺ C₁₆H₂₃NNaO₆: 348.15; found: 348.19.

### Cap-193

### Step c

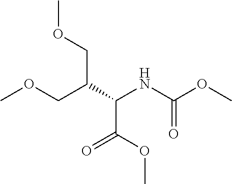

A reaction mixture of Cap-193, Step b (0.16 g), dimethyl dicarbonate (0.13 g) and 10% Pd/C (0.026 g) in EtOAc was stirred under H₂ at room temperature for 2 hrs. The reaction mixture was filtered and concentrated to yield the methyl carbamate Cap-193, Step c. LC-MS: Anal. Calcd. For [M+Na]⁺ C₁₀H₁₉NNaO₆: 272.12; found: 272.07.

### Cap-193

To a solution of ester Cap-193, Step c in THF (1 mL) and MeOH (0.25 mL) was added 1 N NaOH (1 mL). The reaction mixture was stirred at room temperature for 2 hrs. The reaction mixture was concentrated and diluted with EtOAc and 1 N HCl. The aqueous phase was extracted with EtOAc, and the combined organic phase was washed with sat. NaCl, dried over anhydrous Na₂SO₄, filtered and concentrated to yield Cap-193 (0.082 g). ¹H NMR (400 MHz, CDCl₃) 5.99 (1H, d, J=8.56 Hz), 4.57 (1H, dd, J=8.56, 3.27 Hz), 3.67 (3H, s), 3.49 (2H, d, J=4.28 Hz), 3.45-3.44 (2 H, m), 3.26-3.35 (6H, m). LC-MS: Anal. Calcd. For [M+Na]⁺ C₉H₁₇NNaO₆: 258.11; found: 258.13.

### Cap-194

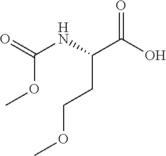

Piperidine (1.0 mL, 10 mmol) was added to a solution of (S)-2-(((9H-fluoren-9-yl)methoxy)carbonylamino)-4-methoxybutanoic acid (0.355 g, 1 mmol) in DMF (3 mL), and the mixture was stirred at rt for 3 h. The volatiles were removed and the residue was partitioned between sat. NaHCO₃ (aq.) (5 mL) and EtOAc (5 mL). The aqueous layer was further washed with EtOAc and Et₂O. To the aqueous solution was added Na₂CO₃ (212 mg, 2.0 mmol) followed by methyl chloroformate (0.16 mL, 2.0 mmol) and the reaction mixture was stirred at rt for 16 h. The reaction mixture was acidified with 1 N HCl (aq.) until pH<7 and then extracted with EtOAc (2×10 mL). The combined organic layers were dried (Na₂SO₄), filtered and concentrated. The residue was purified by flash silica chromatography (EtOAc/hexanes, gradient from 20% to 70%) to yield (S)-4-methoxy-2-(methoxycarbonylamino)butanoic acid (Cap-194) (91.5 mg) as viscous colorless oil. LC-MS retention time=0.61 min; m/z 214 [M+Na]⁺. (Column: PHENOMENEX® Luna 3.0×50 mm S10. Solvent A=90% Water:10% Methanol:0.1% TFA. Solvent B=10% Water:90% Methanol: 0.1% TFA. Flow Rate=4 mL/min. Start % B=0. Final % B=100. Gradient Time=3 min. Wavelength=220). ¹H NMR (400 MHz, chloroform-d) δ ppm 7.41 (br. s., 1H), 5.74-6.02 (m, 1H), 4.32-4.56 (m, 1H), 3.70 (s, 3H), 3.54 (t, J=5.0 Hz, 2H), 3.34 (s, 3H), 1.99-2.23 (m, 2H).

### Cap-195

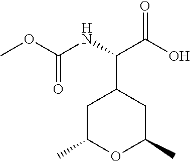

### Cap-195

### Step a

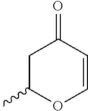

Reference: S. Danishefsky and J. F. Kerwin, Jr J. Org. Chem., 1982, 47, 1597.

Boron trifluoride etherate (3.81 mL, 30.5 mmol) was added dropwise to a stirred and cooled (−78° C.) solution of (E)-(4-methoxybuta-1,3-dien-2-yloxy)trimethylsilane (5.0 g, 29 mmol) and acetaldehyde (3.28 mL, 58.0 mmol) in diethyl ether (100 mL) under nitrogen. The reaction was stirred at −78° C. for 2.5 h and then quenched with sat. aq. NaHCO₃ (40 mL), allowed to warm to RT and stirred ON. The layers were separated and the aqueous layer was extracted with diethyl ether (2×50 mL). The combined organic layers were dried (MgSO₄), filtered and concentrated to a yellow/orange oil. The crude oil was purified with a Biotage® Horizon (110 g SiO₂, 25-40% EtOAc/hexanes) to yield racemic 2-methyl-2H-pyran-4(3H)-one (Cap-195, step a) (2.2 g) as a yellow oil. ¹H NMR (400 MHz, CDCl₃-d) δ ppm 7.35 (d, J=6.0 Hz, 1H), 5.41 (dd, J=6.0, 1.0 Hz, 1H), 4.51-4.62 (m, 1H), 2.41-2.57 (m, 2H), 1.47 (d, J=6.3 Hz, 3H).

### Cap-195

### Step b

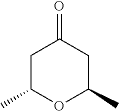

Reference: Reddy, D. S.; Vander Velde, D.; Aube, J. J. Org. Chem. 2004, 69, 1716-1719.

A solution of 1.6M methyllithium in diethyl ether (20.9 mL, 33.4 mmol) was added to a stirred slurry of copper(I) iodide (4.25 g, 22.30 mmol) in diethyl ether (30 mL) at 0° C. and under nitrogen. The reaction was stirred at 0° C. for 20 min and then racemic 2-methyl-2H-pyran-4(3H)-one (1.25 g, 11.2 mmol) in diethyl ether (12.0 mL) was added over 10 min. The reaction was allowed to warm to RT and stirred 2 h. The reaction mixture was poured into sat NH₄Cl (aq) and stirred 20 min. The solution was extracted with diethyl ether (4×60 mL) and the combined organics were washed with brine (˜80 mL), dried (MgSO₄), filtered and concentrated to yield racemic (2R,6R)-2,6-dimethyldihydro-2H-pyran-4(3H)-one (Cap-195, step b) (1.34 g) as an orange oil. ¹H NMR (400 MHz, CDCl₃) δ ppm 4.28-4.39 (m, 2H), 2.57 (dd, J=4.8, 1.5 Hz, 1H), 2.53 (dd, J=4.9, 1.4 Hz, 1H), 2.26 (dd, J=6.5, 1.5 Hz, 1H), 2.23 (dd, J=6.5, 1.5 Hz, 1H), 1.28 (d, J=6.3 Hz, 6H).

### Cap-195

### Step c

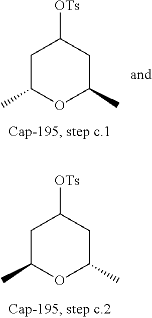

Sodium borohydride (0.354 g, 9.36 mmol) was added in portions to a stirred solution of racemic (2R,6R)-2,6-dimethyldihydro-2H-pyran-4(3H)-one (Cap-195, step b) (1.2 g, 9.4 mmol) in MeOH (30 mL) at 0° C. The solution was stirred 10 min at 0° C., warmed to RT and stirred 1 h. The reaction was poured into sat NH₄Cl (−50 mL), stirred 20 min and then partially concentrated (to ˜½ volume). A precipitate formed and water was added until homogeneous and then the solution was extracted with DCM (3×60 mL). The aqueous layer was acidified with 1N HCl and then extracted with DCM (3×60 mL). The combined organics were dried with Na₂SO₄, filtered and concentrated to form a cloudy yellow oil (1.08 g). The crude oil was dissolved into DCM (8.0 mL) and then p-tosyl-Cl (2.68 g, 14.0 mmol) and pyridine (1.51 mL, 18.7 mmol) were added and the reaction was allowed to stir at RT for 2.5d. The reaction was diluted with sat NH₄Cl (˜60 mL) and extracted with DCM (3×30 mL). The combined organic phase was dried (MgSO₄), filtered and concentrated to a brown oil. The oil was purified on a Biotage® Horizon (80 g SiO₂, 10-25% EtOAc/hexanes) to yield racemic (2R,6R)-2,6-dimethyltetrahydro-2H-pyran-4-yl-4-methylbenzenesulfonate (Cap-195, step c) (1.63 g) as a viscous clear colorless oil. LC-MS retention time 3.321 min; m/z 284.98 [M+H]⁺. LC data was recorded on a Shimadzu LC-10AS liquid chromatograph equipped with a Phenomenex-Luna 3u C18 2.0×50 mm column using a SPD-10AV UV-Vis detector at a detector wave length of 220 nM. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 5% MeOH/95% H₂O/10 mM ammonium acetate and solvent B was 5% H₂O/95% MeOH/10 mM ammonium acetate. MS data was determined using a Micromass Platform for LC in electrospray mode. ¹H NMR (400 MHz, CDCl₃) δ ppm 7.81 (2H, d, J=8.3 Hz), 7.36 (2H, d, J=8.0 Hz), 4.81-4.92 (1H, m), 4.17-4.26 (1H, m), 3.78-3.87 (1H, m), 2.47 (3H, s), 1.91-1.99 (1H, m), 1.78-1.86 (1H, m), 1.65-1.72 (1H, m), 1.46 (1H, ddd, J=12.9, 9.4, 9.3 Hz), 1.20 (6H, dd, J=6.5, 4.8 Hz).

The racemic mixture was separated into the individual enantiomers in multiple injections using chiral preparative SFC purification (Chiralpak AD-H preparative column, 30×250 mm, 5 μm, 10% 1:1 EtOH/heptane in CO₂, 70 mL/min. for 10 min) to yield (2R,6R)-2,6-dimethyltetrahydro-2H-pyran-4-yl-4-methylbenzenesulfonate (Cap-195, step c.1) (577 mg) as the first eluting peak and (2S,65)-2,6-dimethyltetrahydro-2H-pyran-4-yl-4-methylbenzenesulfonate (Cap-195, step c.2) (588 mg) as the second eluting peak. Each enantiomer was isolated as a clear colorless oil which solidified to a white solid upon standing.

### Cap-195

### Step d

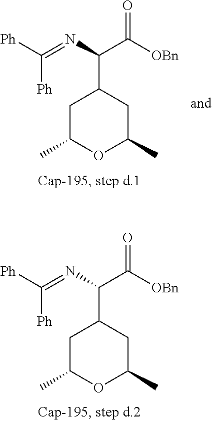

In a 48 mL pressure tube, (2R,6R)-2,6-dimethyltetrahydro-2H-pyran-4-yl-4-methylbenzenesulfonate (Cap-195, step c.1) (575 mg, 2.02 mmol) and benzyl 2-(diphenylmethyleneamino)acetate (733 mg, 2.22 mmol) were stirred in THF (2 mL) and toluene (10 mL). The clear colorless solution was flushed with nitrogen and then LiHMDS (1.0M in THF) (2.22 mL, 2.22 mmol) was added and the vessel was sealed and heated at 100° C. for 8 h. The reaction was cooled to RT, poured into ½ sat NH₄Cl (aq) (−50 mL) and extracted with EtOAc (3×30 mL). The combined organic layers were washed with brine, dried (MgSO₄), filtered and concentrated to a crude orange oil. The oil was purified on a Biotage Horizon (40 g SiO₂, 10-25% EtOAc/hexanes) to yield impure desired product (501 mg) as an orange oil. This material was repurified on a Biotage® Horizon (25 g SiO₂, 6-12% EtOAc/hexanes) to yield an ˜1:1 mixture of diastereomers (Cap-195, step d) (306 mg) as a viscous orange oil.

The mixture was separated into the individual diastereomers in multiple injections using chiral preparative SFC purification (Chiralcel OJ-H preparative column, 30×250 mm, 5 μm, 10% 1:1 EtOH/heptane in CO₂ @150 bar, 70 mL/min. for 10 min) to yield (R)-benzyl 2-((2R,6R)-2,6-dimethyltetrahydro-2H-pyran-4-yl)-2-(diphenylmethyleneamino)acetate (Cap-195, step d.1) (124 mg) as the first eluting peak and (S)-benzyl 2-((2R,6R)-2,6-dimethyltetrahydro-2H-pyran-4-yl)-2-(diphenylmethyleneamino)acetate (Cap-195, step d.2) (129 mg) as the second eluting peak. Each diastereomer was isolated as a viscous yellow oil.

Analytical data for (R)-benzyl 2-((2R,6R)-2,6-dimethyltetrahydro-2H-pyran-4-yl)-2-(diphenylmethyleneamino)acetate (Cap-195, step d.1): ¹H NMR (400 MHz, D₄-MeOH) δ ppm 7.57-7.61 (m, 2H), 7.41-7.48 (m, 4H), 7.33-7.40 (m, 7H), 7.03-7.08 (m, 2H), 5.22 (d, J=12.1 Hz, 1H), 5.16 (d, J=12.1 Hz, 1H), 4.09-4.19 (m, 1H), 3.84 (d, J=6.8 Hz, 1H), 3.75-3.83 (m, 1H), 2.53-2.64 (m, 1H), 1.58-1.65 (m, 1H), 1.33-1.43 (m, 1H), 1.26-1.32 (m, 1H), 1.24 (d, J=7.0 Hz, 3H), 1.10 (d, J=6.0 Hz, 3H), 0.98-1.08 (m, 1H). LC-MS retention time 4.28 min; m/z 442.16 [M+H]⁺. LC data was recorded on a Shimadzu LC-10AS liquid chromatograph equipped with a Phenomenex-Luna 3u C18 2.0×50 mm column using a SPD-10AV UV-Vis detector at a detector wave length of 220 nM. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 5% MeOH/95% H₂O/10 mM ammonium acetate and solvent B was 5% H₂O/95% MeOH/10 mM ammonium acetate. MS data was determined using a Micromass Platform for LC in electrospray mode.

Analytical data for (S)-benzyl 2-((2R,6R)-2,6-dimethyltetrahydro-2H-pyran-4-yl)-2-(diphenylmethyleneamino)acetate (Cap-195, step d.2): ¹H NMR (400 MHz, D₄-MeOH) δ ppm 7.57-7.61 (m, 2H), 7.41-7.50 (m, 4H), 7.33-7.40 (m, 7H), 7.04-7.08 (m, 2H), 5.22 (d, J=12.1 Hz, 1H), 5.16 (d, J=12.1 Hz, 1H), 4.20 (qd, J=6.4, 6.3 Hz, 1H), 3.86 (d, J=6.5 Hz, 1H), 3.74-3.83 (m, 1H), 2.53-2.64 (m, 1 H), 1.60 (td, J=12.7, 5.6 Hz, 1H), 1.38-1.51 (m, 2H), 1.26 (d, J=7.0 Hz, 3H), 1.04 (d, J=6.0 Hz, 3H), 0.79-0.89 (m, 1H). LC-MS retention time 4.27 min; m/z 442.17 [M+H]⁺. LC data was recorded on a Shimadzu LC-10AS liquid chromatograph equipped with a Phenomenex-Luna 3u C18 2.0×50 mm column using a SPD-10AV UV-Vis detector at a detector wave length of 220 nM. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 5% MeOH/95% H₂O/10 mM ammonium acetate and solvent B was 5% H₂O/95% MeOH/10 mM ammonium acetate. MS data was determined using a Micromass Platform for LC in electrospray mode.

### Cap-195

### Step e

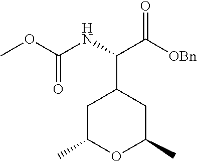

(S)-Benzyl 2-((2R,6R)-2,6-dimethyltetrahydro-2H-pyran-4-yl)-2-(diphenylmethyleneamino)acetate (Cap-195, step d.2) (129.6 mg, 0.294 mmol) was dissolved in THF (2 mL) and then treated with 2N HCl (1.0 mL, 2.1 mmol) in water. The reaction was stirred for 2 h and then concentrated under a stream of nitrogen overnight. The crude residue was dissolved in DCM (2 mL) and DIPEA (0.21 mL, 1.2 mmol) and then treated with methyl chloroformate (0.032 mL, 0.41 mmol) and stirred at RT for 4 h. The reaction was diluted with water (˜2.5 mL) and extracted with DCM (4×2 mL). The combined organic phase was concentrated under a stream of nitrogen overnight and the residue was purified by Biotage® Horizon (4 g SiO₂, 10-50% EtOAc/hexanes) to yield (S)-benzyl 2-((2R,6R)-2,6-dimethyltetrahydro-2H-pyran-4-yl)-2-(methoxycarbonylamino)acetate (Cap-195, step e) (56 mg) as a colorless glass. LC-MS retention time 3.338 min; m/z 335.99 [M+H]⁺. LC data was recorded on a Shimadzu LC-10AS liquid chromatograph equipped with a Phenomenex-Luna 3u C18 2.0×50 mm column using a SPD-10AV UV-Vis detector at a detector wave length of 220 nM. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 5% MeOH/95% H₂O/10 mM ammonium acetate and solvent B was 5% H₂O/95% MeOH/10 mM ammonium acetate. MS data was determined using a Micromass Platform for LC in electrospray mode. ¹H NMR (400 MHz, D₄-MeOH) δ ppm 7.29-7.42 (m, 5H), 5.28 (d, J=12.0 Hz, 1H), 5.09 (d, J=12.0 Hz, 1H), 4.10-4.20 (m, 2H), 3.68-3.78 (m, 1H), 3.65 (s, 3H), 2.22-2.36 (m, 1H), 1.42-1.54 (m, 2H), 1.29-1.38 (m, 1H), 1.17 (d, J=6.8 Hz, 3H), 1.04 (d, J=6.0 Hz, 3H), 0.89-1.00 (m, 1H).

### Cap-195

(S)-Benzyl 2-((2R,6R)-2,6-dimethyltetrahydro-2H-pyran-4-yl)-2-(methoxycarbonylamino)acetate (Cap-195, step e) (56 mg, 0.167 mmol) was dissolved in MeOH (4 mL) and then treated with 10% Pd/C (12 mg, 0.012 mmol). The reaction mixture was vacuum flushed with nitrogen (4×) and then with hydrogen (4×) and stirred under a balloon of hydrogen overnight. The reaction was filtered through Celite® and concentrated to yield (S)-2-((2R,6R)-2,6-dimethyltetrahydro-2H-pyran-4-yl)-2-(methoxycarbonylamino)acetic acid (Cap-195) (41 mg) as a colorless oil. ¹H NMR (400 MHz, D₄-MeOH) δ ppm 4.22 (quin, J=6.4 Hz, 1H), 4.04-4.11 (m, 1H), 3.78-3.87 (m, 1H), 3.66 (s, 3H), 2.26-2.39 (m, 1H), 1.63 (d, J=13.1 Hz, 1H), 1.51-1.60 (m, 1H), 1.42-1.49 (m, 1H), 1.27 (d, J=7.0 Hz, 3H), 1.11 (d, J=6.3 Hz, 3H), 0.97-1.08 (m, 1H).

Note: The absolute stereochemistry of Cap-195 was determined by single crystal X-ray analysis of an amide analog prepared from an epimer of Cap-195 ((R)-2-((2R,6R)-2,6-dimethyltetrahydro-2H-pyran-4-yl)-2-((methoxycarbonyl)amino)acetic acid) and (S)-1-(naphthalen-2-yl)ethanamine.

### Cap-196.1 & Cap-196.2

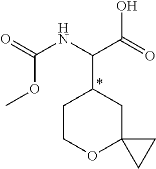

### Cap-196

### Step a

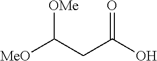

A mixture of methyl 3,3-dimethoxypropanoate (10 g, 67.5 mmol), LiOH (8.08 g, 337 mmol) in a solvent with 40 mL of MeOH, 40 mL of THF and 40 mL of water was heated at 80° C. for 2 hours. The mixture was then cooled down to room temperature and acidified with 1 N HCl aqueous solution (pH>3). The mixture was then extracted with CH₂Cl₂ (3×). The combined organic layers were dried with MgSO₄ and concentrated to give Cap-196, step a as a clear oil (6.3 g). The product was used in the next reaction without further purification. ¹H NMR (500 MHz, CDCl₃) δ 4.82 (t, J=5.8 Hz, 1H), 3.36 (s, 6H), 2.69 (d, J=5.8 Hz, 2H); ¹³C NMR (125 MHz, CDCl₃) δ 175.22, 101.09, 53.68, 38.76.

### Cap-196

### Step b

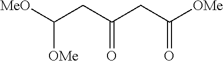

To a solution of Cap-196, step a (4.55 g, 33.9 mmol) in 40 mL of THF was added the suspension of N,N′-carbonyldiimidazole (6.60 g, 40.7 mmol) in 40 mL of THF dropwise. The solution turned yellow and gas evolution was observed. The mixture was stirred at room temperature for 2 hours. At the same time, another flask with monomethyl monopotassium malonate (7.95 g, 50.9 mmol) and magnesium chloride (3.55 g, 37.3 mmol) in 80 mL of THF was stirred at room temperature for 2 hours too. The imidazolide solution was then transferred into the Mg(OOCCH₂COOMe)₂ solution by syringe and the resulting mixture was stirred at room temperature for 16 h. The mixture was then acidified with 60 mL of NaHSO₄ (2M) solution and extracted with EtOAc (3×). The combined organic layers were washed with sat. NaHCO₃ aqueous solution, brine, dried with MgSO₄ and concentrated to give Cap-196, step b as a light purple-colored oil (4.9 g). The oil was used in the next step without further purification. ¹H NMR (500 MHz, CDCl₃) δ 4.75 (t, J=5.5 Hz, 1H), 3.72 (s, 3H), 3.50 (s, 2H), 3.35 (s, 6H), 2.84 (d, J=5.5 Hz, 2H).

### Cap-196

### Step c

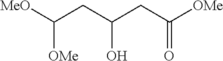

To a solution of Cap-196, step b (4.9 g, 25.8 mmol) in 70 mL of MeOH was slowly added sodium borohydride (1.072 g, 28.3 mmol). The resulting mixture was stirred at room temperature for 3 hours and the quenched with 1N HCl (15 mL). The mixture was then extracted with EtOAc (3×). The combined organic layers were dried with MgSO₄ and concentrated to afford Cap-196, step c as a light yellow oil (4.4 g). The product was used in the next step without further purification. ¹H NMR (400 MHz, CDCl₃) δ 4.60 (t, J=5.5 Hz, 1H), 4.25-4.16 (m, 1H), 3.70 (s, 3H), 3.36 (d, J=1.5 Hz, 6H), 2.52-2.48 (m, 2H), 1.83-1.77 (m, 2H); ¹³C NMR (100 MHz, CDCl₃) δ 172.21, 102.73, 64.61, 53.22, 52.95, 51.30, 41.00, 38.65.

### Cap-196

### Step d

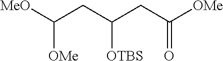

To a solution of Cap-196, step c (4.4 g, 22.89 mmol) in 50 mL of DMF was added imidazole (3.12 g, 45.8 mmol) and TBS—Cl (5.52 g, 36.6 mmol). The resulting mixture was stirred at room temperature for 3 days. The reaction was then diluted with CH₂Cl₂ and washed with water. The organic phase was washed with brine, dried with MgSO₄ and concentrated. The crude product was purified by flash chromatography (silica gel, 0-15% EtOAc/Hex) to afford Cap-196, step d as a clear oil (5.0 g). ¹H NMR (400 MHz, CDCl₃) δ 4.55-4.50 (m, 1H), 4.30-4.21 (m, 1H), 3.67 (s, 3H), 3.32 (d, J=1.5 Hz, 6H), 2.51 (d, J=6.3 Hz, 2H), 1.89-1.77 (m, 2H), 0.88 (s, 9H), 0.08 (d, J=11.0 Hz, 6H); ¹³C NMR (100 MHz, CDCl₃) δ 171.41, 101.24, 65.85, 52.35, 52.09, 51.04, 42.40, 39.92, 25.37, 25.27, 17.55, −3.95, −5.15.

### Cap-196

### Step e

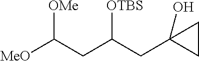

To a solution of Cap-196, step d (5.0 g, 16.31 mmol) in 50 mL of ether in a water bath was added tetraisopropyl titanate (0.971 mL, 3.26 mmol) in 10 mL of ether. The solution turned yellow. Ethylmagnesium bromide (48.9 mL, 48.9 mmol) (1 M in THF) was then added dropwise by a syringe pump over 1 hour. The solution turned dark brown with some precipitate. The mixture was then stirred in water bath for 2 hours. The mixture was diluted with ether and quenched with sat. NH₄Cl aqueous solution slowly. The resulting white precipitate was filtered off. The filtrate was extracted with Et₂O (3×). The combined organic layers were dried with MgSO₄ and concentrated. The crude product was then purified by flash chromatography (silica gel, 0-20% EtOAc/Hex) to afford Cap-196, step e as a clear oil (4.02 g). ¹H NMR (500 MHz, CDCl₃) δ 4.47 (t, J=5.6 Hz, 1H), 4.21-4.14 (m, 1H), 3.71 (s, 1H), 3.31 (d, J=1.8 Hz, 6H), 2.05-1.88 (m, 3H), 1.66-1.58 (m, 1H), 0.90 (s, 9H), 0.83-0.76 (m, 1H), 0.71-0.65 (m, 1H), 0.47 (m, 1H), 0.40-0.34 (m, 1H), 0.12 (d, J=11.0 Hz, 6H); ¹³C NMR (100 MHz, CDCl₃) δ 102.09, 69.97, 54.44, 52.97, 52.84, 43.27, 40.00, 25.92, 17.96, 14.04, 12.06, −4.32, −4.61.

### Cap-196

### Step f

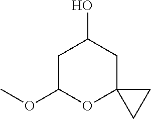

A solution of Cap-196, step e (4.02 g, 13.20 mmol) and p-toluenesulfonic acid monohydrate (3.01 g, 15.84 mmol) in 120 mL of MeOH was stirred at room temperature overnight. To the mixture was added 100 mL of sat. NaHCO₃ solution and the mixture was extracted with CH₂Cl₂ (3×). The combined organic layers were dried with MgSO₄ and concentrated. The crude product was quickly purified by flushing it through a silica gel bed with 70% EtOAc/Hex to afford Cap-196, step f as a clear oil (1.7 g). ¹H NMR (500 MHz, CDCl₃) δ 4.79 (t, J=3.7 Hz, 1H), 4.59 (dd, J=5.3, 2.9 Hz, 1H), 4.30-4.22 (m, 1H), 4.03 (br. s., 1H), 3.37 (s, 3H), 3.31 (s, 3H), 2.09-2.03 (m, 1H), 2.00 (dtd, J=13.1, 4.0, 1.5 Hz, 1H), 1.86 (dd, J=13.1, 3.7 Hz, 1H), 1.81-1.61 (m, 7H), 0.94-0.87 (m, 1H), 0.83-0.77 (m, 1H), 0.74-0.69 (m, 1H), 0.65-0.56 (m, 2H), 0.48-0.40 (m, 2H), 0.37-0.30 (m, 1H); ¹³C NMR (100 MHz, CDCl₃) δ 100.86, 100.27, 65.75, 64.35, 56.24, 55.92, 53.80, 52.35, 40.86, 39.92, 39.28, 38.05, 12.10, 12.06, 9.91, 9.37.

### Cap-196

### Step g

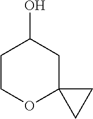

To a solution of Cap-196, step f (1.7 g, 10.75 mmol) in 20 mL of CH₂Cl₂ was added bis(trimethylsilyl)trifluoroacetamide (2.139 mL, 8.06 mmol). The mixture was stirred at room temperature for 2 hours. The mixture was then cooled down to −10° C. Triethylsilane (6.87 mL, 43.0 mmol) was added followed by boron trifluoride ether complex (3.40 mL, 26.9 mmol) dropwise. The mixture turned light purple immediately upon adding boron trifluoride ether complex. The mixture was then allowed to warm to 0° C. slowly and stirred at 0° C. for 30 mins. The reaction was then quenched with water and extracted with EtOAc (3×). The combined organic layers were dried with MgSO₄ and concentrated. The crude product was quickly purified by flushing it through a silica gel bed with 70% EtOAc/Hex afford Cap-196, step g as a clear oil (1.5 g). ¹H NMR (500 MHz, CDCl₃) δ 6.43 (br. s., 1H), 3.96 (tt, J=9.5, 4.7 Hz, 1H), 3.86 (dt, J=11.5, 4.0 Hz, 1H), 3.51 (td, J=11.1, 2.7 Hz, 1H), 1.98-1.84 (m, 2H), 1.66-1.50 (m, 2H), 0.86-0.79 (m, 1H), 0.66-0.59 (m, 1H), 0.53-0.46 (m, 1H), 0.34 (m, 1H); ¹³C NMR (100 MHz, CDCl₃) δ 67.41, 64.37, 57.99, 41.28, 35.22, 11.74, 11.32.

### Cap-196

### Step h

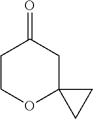

To a solution of oxalyl chloride (1.090 mL, 12.45 mmol) in 30 mL of CH₂Cl₂ at −78° C. was added DMSO (1.767 mL, 24.90 mmol) in 20 mL of CH₂Cl₂ dropwise. The mixture was stirred for 20 min, and Cap-196, step g (1.33 g, 10.38 mmol) in 20 mL of CH₂Cl₂ was added dropwise. The mixture was stirred at −78° C. for 20 mins Et₃N (7.52 mL, 54.0 mmol) was then added and the mixture was warmed slowly to room temperature over 30 mins. The mixture was then quenched with water and extracted with CH₂Cl₂ (3×). The organic layers were combined and dried with MgSO₄ and concentrated to give Cap-196, step h as a clear oil (1.3 g). The crude product was used in the next step without purification. ¹H NMR (500 MHz, CDCl₃) δ 3.95 (t, J=6.0 Hz, 2H), 2.55-2.50 (m, 2H), 2.46 (s, 2H), 0.84 (m, 2H), 0.50 (m, 2H).

### Cap-196

### Step i

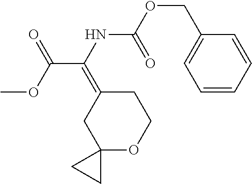

To a solution of methyl 2-(((benzyloxy)carbonyl)amino)-2-(dimethoxyphosphoryl)acetate (3.41 g, 10.30 mmol) in 20 mL of THF at −20° C. was added 1,1,3,3-tetramethylguanidine (2.85 mL, 22.67 mmol). The resulting mixture was stirred at −20° C. for 1 hour. Cap-196, step h (1.3 g, 10.30 mmol) in 10 mL of THF was then added. The resulting brown mixture was stirred at room temperature for 6 days. The reaction was then concentrated and the crude product was purified by flash chromatography (silica gel, 0-25% EtOAc/Hex) to afford Cap-196, step i (mixture of isomers) as a white solid (850 mg). LC/MS: Anal. Calcd. for [M+H]⁺ C₁₈H₂₂NO₅ 332.15; found 332.14; ¹H NMR (500 MHz, CDCl₃) δ 7.45-7.29 (m, 5H), 6.09-5.81 (m, 1H), 5.18-5.08 (m, 2H), 3.88-3.49 (m, 5H), 3.06-2.82 (m, 2H), 2.52-2.36 (m, 2H), 0.82-0.64 (m, 2H), 0.58-0.32 (m, 2H).

### Cap-196.1 and Cap-196.2

### Step j

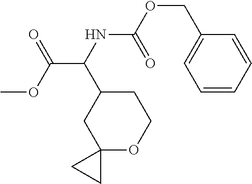

A solution of Cap-196, step i (mixture of isomers) (730 mg, 2.203 mmol) in 5 mL of MeOH in a 500 mL hydrogenation pressure tube was bubbled with N₂ for 30 mins. To the mixture was added (−)-1,2-bis((2S,5S)-2,5-dimethylphospholano)ethane(cyclooctadiene) rhodium (I) tetrafluoroborate (24.51 mg, 0.044 mmol) and the bottle was then put on a Parr shaker and hydrogenated at 60 psi for 3 days. The mixture was then concentrated. The crude product was then separated by chiral HPLC (Chiralpak AD column, 21×250 mm, 10 um) eluting with 85% 0.1% diethylamine/Heptane-15% EtOH at 15 mL/min to afford Cap-196.1, step j (220 mg) (first eluting fraction) and Cap-196.2, step j (290 mg) (second eluting fraction) as clear oils. The absolute stereochemistry of the isomers was not determined

Cap-196.1, step j: LC/MS: Anal. Calcd. for [M+Na]⁺ C₁₈H₂₃NNaO₅ 356.15; found 356.16; ¹H NMR (500 MHz, CDCl₃) δ 7.41-7.28 (m, 5H), 5.34 (d, J=8.9 Hz, 1H), 5.10 (s, 2H), 4.37 (dd, J=9.0, 5.0 Hz, 1H), 3.89-3.82 (m, 1H), 3.75 (s, 3H), 3.48 (td, J=11.1, 3.1 Hz, 1H), 2.29-2.17 (m, 1H), 1.96 (t, J=12.7 Hz, 1H), 1.57-1.43 (m, 2H), 1.07-0.98 (m, 1H), 0.87-0.78 (m, 1H), 0.66-0.56 (m, 1H), 0.56-0.47 (m, 1H), 0.37-0.27 (m, 1H);

Cap-196.2, step j: LC/MS: Anal. Calcd. for [M+Na]⁺ C₁₈H₂₃NNaO₅ 356.15; found 356.17; ¹H NMR (500 MHz, CDCl₃) δ 7.40-7.28 (m, 5H), 5.33 (d, J=8.5 Hz, 1H), 5.10 (s, 2H), 4.36 (dd, J=8.9, 5.8 Hz, 1H), 3.86 (dd, J=11.0, 3.1 Hz, 1H), 3.74 (s, 3H), 3.53-3.43 (m, 1H), 2.25-2.14 (m, 1H), 1.94 (t, J=12.5 Hz, 1H), 1.67-1.44 (m, 2H), 0.97-0.90 (m, 1H), 0.86-0.79 (m, 1H), 0.66-0.57 (m, 1H), 0.53-0.44 (m, 1H), 0.33-0.24 (m, 1H).

### Cap-196.1

### Step k

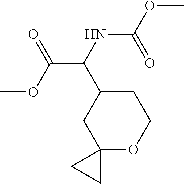

To a solution of Cap-196.1, step j (210 mg, 0.630 mmol) in 10 mL of MeOH in a hydrogenation flask was added dimethyl dicarbonate (0.135 mL, 1.260 mmol) and Pd/C (33.5 mg, 0.031 mmol). The flask was put on a Parr shaker and the mixture was hydrogenated at 50 psi for 4 hours. The mixture was then filtered through diatomaceous earth (Celite®) and the filtrate was concentrated to afford Cap-196.1, step k as a clear oil (165 mg). LC/MS: Anal. Calcd. for [M+H]⁺ C₁₂H₂₀NO₅ 258.13; found 258.16; ¹H NMR (500 MHz, CDCl₃) δ 5.39 (d, J=8.5 Hz, 1H), 4.30 (dd, J=8.9, 5.2 Hz, 1H), 3.84-3.78 (m, 1H), 3.70 (s, 3H), 3.63 (s, 3H), 3.47-3.39 (m, 1H), 2.23-2.12 (m, 1H), 1.91 (t, J=12.5 Hz, 1H), 1.49-1.39 (m, 2H), 0.97 (dd, J=13.1, 2.4 Hz, 1H), 0.81-0.74 (m, 1H), 0.61-0.52 (m, 1H), 0.46 (dt, J=10.1, 6.0 Hz, 1H), 0.32-0.24 (m, 1H).

### Cap-196.1

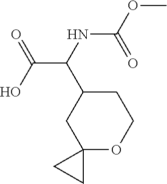

To a mixture of Cap-196.1, step k (165 mg, 0.641 mmol) in 2 mL of THF and 1 mL of water was added LiOH (1 mL, 2.0 mmol) (2 M aqueous). The resulting mixture was stirred at room temperature overnight. The mixture was then washed with ether (1 mL). The aqueous phase was acidified with 1 N HCl aq. solution and extracted with ether (6×). The combined organic layers were dried with MgSO₄ and concentrated to afford Cap-196.1 as a white solid (150 mg). LC/MS: Anal. Calcd. for [M+H]⁺ C₁₁H₁₈NO₅ 244.12; found 244.09; ¹H NMR (500 MHz, CDCl₃-d) δ 5.27 (d, J=8.9 Hz, 1H), 4.39 (dd, J=8.5, 4.9 Hz, 1H), 3.94-3.86 (m, 1H), 3.70 (s, 3H), 3.56-3.46 (m, 1H), 2.36-2.24 (m, 1H), 2.01 (t, J=12.7 Hz, 1H), 1.63-1.48 (m, 2H), 1.14-1.05 (m, 1H), 0.92-0.80 (m, 1H), 0.69-0.60 (m, 1H), 0.58-0.49 (m, 1H), 0.40-0.31 (m, 1H).

### Cap 196.2

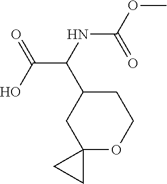

Cap-196.2 was synthesized from Cap-196.2, step j according to the procedure described for Cap-196.1. LC/MS: Anal. Calcd. for [M+H]⁺ C₁₁H₁₈NO₅ 244.12; found 244.09; ¹H NMR (500 MHz, CDCl₃) δ 5.27 (d, J=8.9 Hz, 1H), 4.38 (dd, J=8.2, 4.9 Hz, 1H), 3.91 (dd, J=11.1, 3.2 Hz, 1H), 3.69 (s, 3H), 3.52 (t, J=11.0 Hz, 1H), 2.34-2.23 (m, 1H), 2.07-1.97 (m, 1H), 1.72-1.61 (m, 1H), 1.54 (qd, J=12.6, 4.7 Hz, 1H), 1.04-0.96 (m, 1H), 0.90-0.82 (m, 1H), 0.68-0.61 (m, 1H), 0.56-0.49 (m, 1H), 0.39-0.30 (m, 1H).

### Cap-197

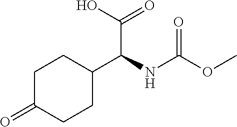

### Cap-197

### Step a

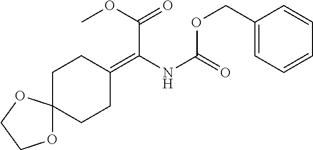

A solution of 1,4-dioxaspiro[4.5]decan-8-one (15 g, 96 mmol) in EtOAc (150 mL) was added to a solution of methyl 2-(benzyloxycarbonylamino)-2-(dimethoxyphosphoryl)acetate (21.21 g, 64.0 mmol) in 1,1,3,3-tetramethylguanidine (10.45 mL, 83 mmol) and EtOAc (150 mL). The resulting solution was the stirred at ambient temperature for 72 h and then it was diluted with EtOAc (25 mL). The organic layer was washed with 1N HCl (75 mL), H₂O (100 mL) and brine (100 mL), dried (MgSO₄), filtered and concentrated. The residue was purified via Biotage (5% to 25% EtOAc/Hexanes; 300 g column). The combined fractions containing the product were then concentrated under vacuum and the residue was re-crystallized from hexanes/EtOAc to give white crystals that corresponded to methyl 2-(benzyloxycarbonylamino)-2-(1,4-dioxaspiro[4.5]decan-8-ylidene)acetate (6.2 g). ¹H NMR (400 MHz, CDCl₃-d) δ ppm 7.30-7.44 (5H, m), 6.02 (1H, br. s.), 5.15 (2H, s), 3.97 (4H, s), 3.76 (3H, br. s.), 2.84-2.92 (2H, m), 2.47 (2H, t, J=6.40 Hz), 1.74-1.83 (4H, m). LC (Cond. OL1): Rₜ=2.89 min. LC/MS: Anal. Calcd. For [M+Na]⁺ C₁₉H₂₃NNaO₆: 745.21; found: 745.47.

### Cap-197

### Step b

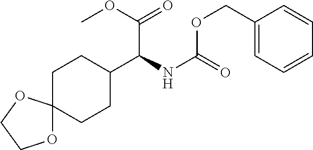

Ester Cap-197, step b was prepared from alkene Cap-197, step a according to the method of Burk, M. J.; Gross, M. F. and Martinez J. P. (J. Am. Chem. Soc., 1995, 117, 9375-9376 and references therein): A 500 mL high-pressure bottle was charged with alkene Cap-197, step a (3.5 g, 9.68 mmol) in degassed MeOH (200 mL) under a blanket of N₂. The solution was then charged with (−)-1,2-Bis((2S,5S)-2,5-dimethylphospholano)ethane(cyclooctadiene)rhodium (I) tetrafluoroborate (0.108 g, 0.194 mmol) and the resulting mixture was flushed with N₂ (3×) and charged with H₂ (3×). The solution was shaken vigorously under 70 psi of H₂ at ambient temperature for 72 h. The solvent was removed under reduced pressure and the remaining residue was taken up in EtOAc. The brownish solution was then filtered through a plug of Silica Gel and eluted with EtOAc. The solvent was concentrated under vacuum to afford a clear oil corresponding to Cap-197, step b (3.4 g). ¹H NMR (500 MHz, CDCl₃-d) δ ppm 7.28-7.43 (5H, m), 5.32 (1H, d, J=9.16 Hz), 5.06-5.16 (2H, m), 4.37 (1H, dd, J=9.00, 5.04 Hz), 3.92 (4H, t, J=3.05 Hz), 3.75 (3H, s), 1.64-1.92 (4 H, m), 1.37-1.60 (5H, m). LC (Cond. OL1): Rₜ=1.95 min. LC/MS: Anal. Calcd. For [M+H]⁺ C₁₉H₂₆NO₆: 364.18; found: 364.27.

### Cap-197

### Step c

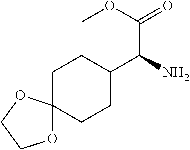

Cap-197, step b (6.68 g, 18.38 mmol) was dissolved in MeOH (150 mL) and charged with Pd/C (0.039 g, 0.368 mmol) and the suspension was placed under 1 atm of H₂. The reaction mixture was stirred at rt for 6 h and filtered though a plug of diatomaceous earth (Celite®) and volatiles were removed under reduced pressure. An amber oil corresponding to Cap-197, step c (3.8 g) was recovered and used without further purification. ¹H NMR (400 MHz, CDCl₃-d) δ 3.92 (br. s., 4H), 3.71 (s, 3H), 3.31 (d, J=4.0 Hz, 1H), 1.87-1.44 (m, 9H). ¹³C NMR (101 MHz, CDCl₃-d) δ 176.1, 108.7, 64.5 (2C), 59.1, 52.0, 41.1, 34.7, 34.6, 27.2, 25.4.

### Cap-197

### Step d

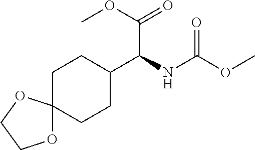

Methyl chloroformate (2.57 mL, 33.1 mmol) was added to a solution of Cap 197, step c (3.8 g, 16.57 mmol) and DIEA (23.16 mL, 133 mmol) in CH₂Cl₂ (200 mL). The resulting solution was stirred at rt for 3 h and volatiles were removed under reduced pressure. The residue was purified via Biotage (30% EtOAc/Hex; 160 g column) An amber oil corresponding to Cap-197, step d (3 g) was recovered. ¹H NMR (500 MHz, CDCl₃-d) δ 5.24 (d, J=8.5 Hz, 1H), 4.34 (dd, J=8.9, 4.9 Hz, 1H), 3.92 (s, 4H), 3.74 (s, 3H), 3.67 (s, 3H), 1.89-1.73 (m, 3H), 1.67 (d, J=12.5 Hz, 1H), 1.62-1.33 (m, 5H). ¹³C NMR (126 MHz, CDCl₃-d) 172.4, 156.7, 108.1, 64.2, 64.2, 57.7, 52.3, 52.2, 39.6, 34.2 (2C), 26.5, 25.0.

### Cap-197

### Step e

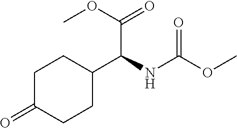

Cap-197, step d (1.15 g, 4.00 mmol) was dissolved in THF (50 mL) followed by sequential addition of water (30 mL), glacial AcOH (8.02 mL, 140 mmol) and dichloroacetic acid (1.985 mL, 24.02 mmol). The mixture was stirred overnight at room temperature and the reaction was quenched by slow addition of solid sodium carbonate with vigorous stirring until the release of gas was no longer visible. Crude product was extracted into 10% ethyl acetate-dichloromethane and the organic layers were combined, dried (MgSO₄), filtered and concentrated. The residue was purified via Biotage (0 to 30% EtOAc/Hex; 40 g column) and a clear oil corresponding to Cap-197, step e (0.72 g) was recovered. ¹H NMR (500 MHz, CDCl₃-d) δ 5.36 (d, J=8.2 Hz, 1H), 4.46 (dd, J=8.4, 5.0 Hz, 1H), 3.77 (s, 3H), 3.68 (s, 3H), 2.46-2.39 (m, 2H), 2.38-2.29 (m, 2H), 2.09-2.03 (m, 1H), 1.96-1.88 (m, 1H), 1.64-1.51 (m, 2H). ¹³C NMR (126 MHz, CDCl₃-d) δ 210.1, 171.9, 156.7, 57.2, 52.5 (2C), 40.2, 40.2, 39.4, 28.7, 27.6.

### Cap-197

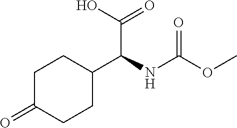

A solution of Cap-197, step e (0.68 g, 2.80 mmol) in THF (7.5 mL) and MeOH (7.50 mL) was cooled to 0° C. 2N aq. NaOH (1.9 mL, 3.80 mmol) was added dropwise and the resulting solution was stirred at room temperature for 2 h. A 1:1 mixture of hexanes:Et₂O (20 mL) was added and the organic layer was discarded. The aqueous layer was then acidified to pH ˜1 with 10% aq. KHSO₄ and the mixture was extracted with EtOAc (2×). The combined organic layers were dried (MgSO₄), filtered and concentrated. A white foam corresponding to Cap-197 (0.55 g)) was recovered and used without further purification. ¹H NMR (500 MHz, DMSO-d₆) δ 12.70 (br. s., 1H), 7.49 (d, J=8.5 Hz, 1H), 4.01 (dd, J=8.2, 6.7 Hz, 1H), 3.54 (s, 3H), 2.45-2.30 (m, 2H), 2.23-2.13 (m, 3H), 1.94-1.79 (m, 3H), 1.57 (qd, J=12.7, 4.1 Hz, 1H), 1.47 (qd, J=12.7, 4.4 Hz, 1H). ¹³C NMR (126 MHz, DMSO-d₆) 210.2, 173.0, 156.8, 57.6, 51.5, 39.7 (2C), 36.9, 28.6, 27.5.

### Cap-198

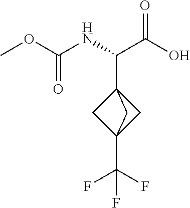

To a mixture of (S)-2-amino-2-(3-(trifluoromethyl)bicyclo[1.1.1]pentan-1-yl)acetic acid (obtained from a commercial source; 0.5151 g, 2.463 mmol) and sodium carbonate (0.131 g, 1.231 mmol) in sodium hydroxide, 1M aq. (2.4 mL, 2.400 mmol) at 0° C. was added methyl carbonochloridate (0.2 mL, 2.59 mmol) dropwise. The reaction was then stirred at room temperature for 4 hr. It was then cooled in an ice/water bath, and diethyl ether (25 mL) was added and stirred and the layers were separated. The aqueous layer was washed with diethyl ether (2×25 mL). The aqueous layer was cooled with an ice-water bath and acidified with 12N HCl to a pH region of 1-2. It was extracted with CH₂Cl₂ (3×50 mL), dried over MgSO₄, and concentrated in vacuo to afford Cap-198 as an off-white solid (480.7 mg) and was used without further purification. ¹H NMR (400 MHz, DMSO-d₆) δ 12.86 (br s, 1H), 7.61 (br d, J=8.0 Hz, 1H), 4.16 (d, J=8.0 Hz, 1H), 3.57 (s, 3H), 2.00 (d, J=8.3 Hz, 3H), 1.93 (d, J=9.3, 3H).

### EXAMPLES

The present disclosure will now be described in connection with certain embodiments which are not intended to limit its scope. On the contrary, the present disclosure covers all alternatives, modifications, and equivalents as can be included within the scope of the claims. Thus, the following examples, which include specific embodiments, will illustrate one practice of the present disclosure, it being understood that the examples are for the purposes of illustration of certain embodiments and are presented to provide what is believed to be the most useful and readily understood description of its procedures and conceptual aspects.

Purity assessment, molecular weight and retention time were conducted according to the following conditions.

### Condition 1 (for a Homogeneity Index Assessment on a Agilent 1200 Series LC System)

Column=Xbridge phenyl ((4.6×150) mm, 3.5 nm)

### Solvent A=Buffer: CH₃CN (95:5)

### Solvent B=Buffer: CH₃CN (5:95)

Buffer=0.05% TFA in H₂O (pH 2.5, adjusted with dilute ammonia)

### Start % B=10

### Final % B=100

Gradient time=12 min
Isocratic time=3 min
Stop time=23 min
Flow Rate=1 mL/min

### Wavelength=220 & 254 nm

### Condition 2 (for a Homogeneity Index Assessment on a Agilent 1200 Series LC System)

### Column=Sunfire C18 ((4.6×150) mm, 3.5 nm)

### Solvent A=Buffer: CH₃CN (95:5)

### Solvent B=Buffer: CH₃CN (5:95)

Buffer=0.05% TFA in H₂O (pH 2.5, adjusted with dilute ammonia)

### Start % B=10

### Final % B=100

Gradient time=12 min
Isocratic time=3 min
Stop time=23 min
Flow Rate=1 mL/min

### Wavelength=220 & 254 nm

### Condition 3 (for a Homogeneity Index Assessment on a Agilent 1200 Series LC System)

Column=Xbridge phenyl ((4.6×150) mm, 3.5 nm)

### Solvent A=Buffer: CH₃CN (95:5)

### Solvent B=Buffer: CH₃CN (5:95)

Buffer=0.05% TFA in H₂O (pH 2.5, adjusted with dilute ammonia)

### Start % B=0

### Final % B=50

Gradient time-1=15 min

### Final % B=100

Gradient time-2=3 min
Isocratic time=5 min
Stop time=28 min
Flow Rate=1 mL/min

### Wavelength=220 & 254 nm

### Condition 4 (for a Homogeneity Index Assessment on a Agilent 1200 Series LC System)

### Column=Sunfire C18 ((4.6×150) mm, 3.5 nm)

### Solvent A=Buffer: CH₃CN (95:5)

### Solvent B=Buffer: CH₃CN (5:95)

Buffer=0.05% TFA in H₂O (pH 2.5, adjusted with dilute ammonia)

### Start % B=0

### Final % B=50

Gradient time-1=15 min

### Final % B=100

Gradient time-2=3 min
Isocratic time=5 min
Stop time=28 min
Flow Rate=1 mL/min

### Wavelength=220 & 254 nm

### Condition 5 (for a Homogeneity Index Assessment on a Agilent 1200 Series LC System)

### Column=Sunfire C18 ((4.6×150) mm, 3.5 nm)

### Solvent A=Buffer: CH₃CN (95:5)

### Solvent B=Buffer: CH₃CN (5:95)

Buffer=0.05% TFA in H₂O (pH 2.5, adjusted with dilute ammonia)

### Start % B=10

### Final % B=100

Gradient time=25 min
Isocratic time=5 min
Stop time=36 min
Flow Rate=1 mL/min

### Wavelength=220 & 254 nm

### Condition 6 (for a Homogeneity Index Assessment on a Agilent 1200 Series LC System)

Column=Xbridge phenyl ((4.6×150) mm, 3.5 nm)

### Solvent A=Buffer: CH₃CN (95:5)

### Solvent B=Buffer: CH₃CN (5:95)

Buffer=0.05% TFA in H₂O (pH 2.5, adjusted with dilute ammonia)

### Start % B=10

### Final % B=100

Gradient time=25 min
Isocratic time=5 min
Stop time=36 min
Flow Rate=1 mL/min

### Wavelength=220 & 254 nm

### Condition 7 (for a Homogeneity Index Assessment on a Agilent 1200 Series LC System)

### Column=Eclipse XDB C18 ((4.6×150) mm, 5 nm)

### Solvent A=20 mM NH₄OAc in H₂O

### Solvent B=CH₃CN

### Start % B=10

### Final % B=100

Gradient time=12 min
Isocratic time=3 min
Stop time=20 min
Flow Rate=1 mL/min

### Wavelength=220 & 254 nm

Condition 8 (LC-MS Analysis on a Agilent LC-1200 Series Coupled with 6140 Single Quad. Mass Spectrometer, ESI+Ve Mode, MS Range: 100-2000)

### Column=Chromolith SpeedROD C18 ((4.6×30) mm, 5 μm)

### Solvent A=MeOH (10%)+0.1% TFA in H₂O (90%)

### Solvent B=MeOH (90%)+0.1% TFA in H₂O (10%)

### Start % B=0

### Final % B=100

Gradient time=2 min
Stop time=1 min
Flow Rate=5 mL/min

### Wavelength=220 nm

Condition 9 (LC-MS Analysis on a Agilent LC-1200 Series Coupled with 6140 Single Quad. Mass Spectrometer, ESI+ve Mode, Ms Range: 100-2000)

### Column=Zorbax SB C18 ((4.6×50) mm, 5 μm)

### Solvent A=MeOH (10%)+0.1% TFA in H₂O (90%)

### Solvent B=MeOH (90%)+0.1% TFA in H₂O (10%)

### Start % B=0

### Final % B=100

Gradient time=2 min
Stop time=3 min
Flow Rate=5 mL/min

### Wavelength=220 nm

Condition 10 (LC-MS Analysis on a Agilent LC-1200 Series Coupled with 6140 Single Quad. Mass Spectrometer, ESI+ve Mode &−ve Mode, MS Range: 100-2000)
Column=Purospher@star RP-18 ((4.0×55) mm, 3 μm)

### Solvent A=ACN (10%)+20 mM NH₄OAc in H₂O (90%)

### Solvent B=ACN (90%)+20 mM NH₄OAc in H₂O (10%)

### Start % B=0

### Final % B=100

Gradient time=2.0 min
Isocratic time=0.5 min
Stop time=3 min
Flow Rate=2.5 mL/min

### Wavelength=220 nm

Condition 11 (LC-MS Analysis on a Agilent LC-1200 Series Coupled with 6140 Single Quad. Mass Spectrometer, ESI+ve Mode &−ve Mode, MS Range: 100-2000)
Column=Purospher@star RP-18 ((4.0×55) mm, 3 μm)

### Solvent A=ACN (10%)+20 mM NH₄OAc in H₂O (90%)

### Solvent B=ACN (90%)+20 mM NH₄OAc in H₂O (10%)

### Start % B=0

### Final % B=100

Gradient time=1.8 min
Isocratic time=1.5 min
Stop time=4 min
Flow Rate=2.5 mL/min

### Wavelength=220 nm

Condition 12 (LC-MS Analysis on a Agilent LC-1200 Series Coupled with 6140 Single Quad. Mass Spectrometer, ESI+ve Mode & −ve Mode, MS Range: 100-2000)
Column=Xbridge phenyl (4.6×30 mm, 3.5 μm)

### Solvent A=CH₃CN (2%)+10 mM NH₄COOH in H₂O (98%)

### Solvent B=CH₃CN (98%)+10 mM NH₄COOH in H₂O (2%)

### Start % B=0

### Final % B=100

Gradient time=1.5 min
Isocratic time=1.7 min
Stop time=4 min
Flow Rate=1.8 mL/min

### Wavelength=220 nm

Condition 13 (LC-MS Analysis on a Agilent LC-1200 Series Coupled with 6330 Ion Trap Mass Spectrometer, ESI+ve Mode & −ve Mode, MS Range: 100-2000)

### Column=Ascentis Express C18 (2.1×50 mm, 2.7 μm)

### Solvent A=CH₃CN (2%)+10 mM NH₄COOH in H₂O (98%)

### Solvent B=CH₃CN (98%)+10 mM NH₄COOH in H₂O (2%)

### Start % B=0

### Final % B=100

Gradient time=1.4 min
Isocratic time=1.6 min
Stop time=4 min
Flow Rate=1.0 mL/min

### Wavelength=220 nm

Condition 14 (LC-MS Analysis on a Agilent LC-1200 Series Coupled with 6330 Ion Trap Mass Spectrometer, ESI+ve Mode & −ve Mode, MS Range: 100-2000)

### Column=Ascentis Express C8 (2.1×50 mm, 2.7 μm)

### Solvent A=CH₃CN (2%)+10 mM NH₄COOH in H₂O (98%)

### Solvent B=CH₃CN (98%)+10 mM NH₄COOH in H₂O (2%)

### Start % B=0

### Final % B=100

Gradient time=1.5 min
Isocratic time=1.5 min
Stop time=4 min
Flow Rate=1.0 mL/min

### Wavelength=220 nm

Condition 15 (LC-MS Analysis on a Agilent LC-1200 Series Coupled with 6330 Ion Trap Mass Spectrometer, ESI+ve Mode & −ve Mode, MS Range: 100-2000)

### Column=Ascentis Express C18 (2.1×50 mm, 2.7 μm)

### Solvent A=CH₃CN (2%)+10 mM NH₄COOH in H₂O (98%)

### Solvent B=CH₃CN (98%)+10 mM NH₄COOH in H₂O (2%)

### Start % B=0

### Final % B=100

Gradient time=1.5 min
Isocratic time=1.5 min
Stop time=4 min
Flow Rate=1.0 mL/min

### Wavelength=220 nm

### Condition 16 (GC-MS Analysis on a Agilent Gcms Module-7890 (GC) 5975C (MSD)

Column=DB-1, 30 m×0.25 mm ID×0.25μ film thickness
Column flow=1.2 mL/min at constant flow of helium
Carrier gas=Helium
Injector temperature=250° C.
Injection volume=1 μL
Split ratio=1:20

### Mass Detector:

Source temperature=230° C.
Quadra pole temperature=150° C.
Column temperature gradient=Initial temperature 50° C. and hold for 1 minute. Ramp rate at 25° C./min up to 300° C. and hold for 5 minutes.
Condition 17 (LC-MS Analysis on a Agilent LC-1200 Series Coupled with 6140 Quadrupole Mass Spectrometer, (ESI+APCI) Multimode+ve Mode & −ve Mode, MS Range: 100-1300)

### Column=YMC PACK TMS (3×50 mm, 3 μm)

### Solvent A=CH₃CN (2%)+10 mM NH₄COOH in H₂O (98%)

### Solvent B=CH₃CN (98%)+10 mM NH₄COOH in H₂O (2%)

### Start % B=0

### Final % B=100

Gradient time=1.5 min
Isocratic time=1.7 min
Stop time=4 min
Flow Rate=1.0 mL/min

### Wavelength=220 nm

Condition 18 (LC-MS Analysis on a Agilent LC-1200 Series Coupled with 6140 Single Quad. Mass Spectrometer, ESI+ve Mode & −ve Mode, MS Range: 100-2000)
Column=Purospher@star RP-18 ((4.0×55) mm, 3 nm)

### Solvent A=ACN (10%)+20 mM NH₄OAc in H₂O (90%)

### Solvent B=ACN (90%)+20 mM NH₄OAc in H₂O (10%)

### Start % B=0

### Final % B=100

Gradient time=2.0 min
Isocratic time=0.5 min
Stop time=4 min
Flow Rate=2.5 mL/min

### Wavelength=220 nm

### Example 1

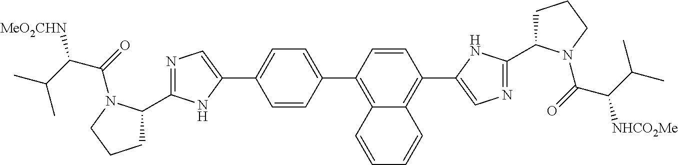

### Example 1

### Step a

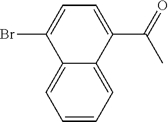

A solution of 1,4-dibromonaphthalene (1.0 g, 3.49 mmol) in toluene (10 mL) was purged with N₂ for 10 minutes. Then tributyl(1-ethoxyvinyl)tin (1.38 g, 3.84 mmol) was added followed by Pd(Ph₃P)₂Cl₂ (251 mg, 0.349 mmol). The reaction mixture was purged with N₂ for 10 minutes and allowed to reflux at 100° C. overnight. The reaction mixture was quenched with saturated KF solution (10 mL) and stirred at room temperature for 2 hrs. The reaction mixture was filtered through a diatomaceous earth (Celite®) plug, and the organic layer was separated and concentrated in vacuo. To the resulting residue, 3 N HCl (20 mL) was added at RT and stirred for 2 hrs. Then the reaction mixture was extracted with EtOAc, washed with brine, dried over Na₂SO₄ and concentrated in vacuo. The crude was purified by flash chromatography (ISCO, EtOAc: petroleum ether, 20:80) to obtain bromide 1a (600 mg). LC/MS (Condition 8): Rₜ=1.99 min. ¹H NMR (CDCl₃, 6=7.26 ppm, 400 MHz): δ 8.75-8.73 (m, 1H), 8.36-8.34 (m, 1H), 7.85 (d, J=7.8, 1H), 7.76 (d, J=7.8, 1H), 7.69-7.65 (m, 2H), 2.76 (s, 3H). LC/MS: Anal. Calcd. for [M+H]⁺ C₁₂H₁₀BrO: 248.98; found 249.0.

### Example 1

### Step b

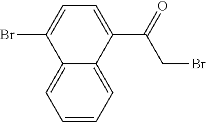

To a stirred solution of bromide 1a (600 mg, 2.40 mmol) in dioxane (10 mL), Br₂ (384 mg, 2.40 mmol) was added at 0° C. and stirred at RT for overnight before quenching with ice. The reaction mixture was extracted with EtOAc, washed with brine, dried over Na₂SO₄ and concentrated in vacuo to obtain crude 2,2-dibromo-1-(4-bromonaphthalen-1-yl)ethanone (780 mg). To the crude dibromoacetyl derivative (400 mg, 0.98 mmol) in ACN (25 mL) was added diethylphosphite (0.12 mL, 0.98 mmol) followed by DIEA (0.17 mL, 0.98 mmol) at room temperature and stirred for 2 hrs. The volatiles were evaporated and water was added to the reaction mixture. Then the reaction mixture was extracted with EtOAc, washed with brine, dried over Na₂SO₄ and concentrated in vacuo to obtain crude 2-bromo-1-(4-bromonaphthalen-1-yl)ethanone (1b) (302 mg) which was used as such in the next step. LC/MS (Condition 8): Rₜ=2.09 min. ¹H NMR (DMSO-d₆, δ=2.50 ppm, 400 MHz): δ 8.44-8.41 (m, 1H), 8.31-8.28 (m, 1H), 8.11 (d, J=7.8, 1H), 8.07 (d, J=7.8, 1H), 7.82-7.76 (m, 2H), 5.06 (s, 2H). LC/MS: Anal. Calcd. for [M+H]⁺ C₁₂H₉Br₂O: 326.89; found 328.8.

### Example 1

### Step c

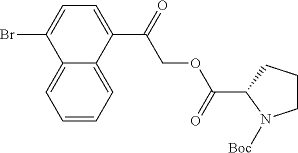

To a stirred solution of dibromide 1b (130 mg, 0.39 mmol) in ACN (20 mL) was added N-Boc-L-proline (85 mg, 0.39 mmol) followed by DIEA (51 mg, 0.39 mmol). The reaction mixture was stirred for 2 hrs at room temperature. The volatiles were evaporated and the reaction mixture was quenched with water. Then the reaction mixture was extracted with EtOAc, washed with brine, dried over Na₂SO₄ and concentrated in vacuo to afford ketoester 1c (150 mg) which was submitted to the next step without purification. LC/MS (Condition 8): Rₜ=2.17 min. ¹H NMR (DMSO-d₆, δ=2.50 ppm, 400 MHz): δ 8.46 (app dd, 1H), 8.28 (d, J=8.0, 1H), 8.06-8.01 (m, 2H), 7.81-7.74 (m, 2H), 5.57-5.42 (m, 2H), 4.36-4.32 (m, 1H), 3.42-3.23 (obscured, 2H), 2.29-2.10 (m, 2H), 1.90-1.77 (m, 2H), 1.35 (s, 9H). LC/MS: Anal. Calcd. for [M+H-Boc]⁺ C₁₂H₁₂BrNO₃: 362.03; found 364.0.

### Example 1

### Step d

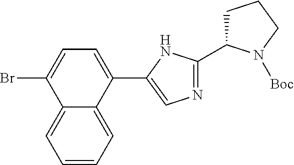

To a solution of ketoester 1c (150 mg, 0.32 mmol) in xylenes (10 mL) was added NH₄OAc (600 mg, 7.79 mmol). The reaction mixture was heated in a pressure tube at 130° C. for overnight. The volatiles were evaporated and the reaction mixture was neutralized with saturated NaHCO₃ solution. Then the reaction mixture was extracted with DCM, washed with water, brine, dried over Na₂SO₄ and concentrated in vacuo. The crude was purified by flash chromatography (ISCO, MeOH: CHCl₃, 5:95) to obtain bromide ld (60 mg). LC/MS (Condition 8): Rₜ=1.73 min. ¹H NMR (DMSO-d₆, δ=2.50 ppm, 400 MHz): δ 12.16/12.07 (br s, 1H), 8.89 (d, J=8.8, 1H), 8.18 (d, J=8.4, 1H), 7.88 (d, J=7.6, 1H), 7.71-7.67 (m, 1H), 7.62-7.58 (m, 2H), 7.47 (br s, 1H), 4.94 (app br d, 0.4H), 4.85 (br s, 0.6H), 3.62-3.50 (m, 1H), 3.43-3.32 (m, 1H), 2.33-2.21 (m, 1H), 2.09-1.86 (m, 3H), 1.43 (s, 4H), 1.20 (s, 5H). LC/MS: Anal. Calcd. for [M+H]⁺ C₂₂H₂₅BrN₃O₂: 442.11; found 442.0.

### Example 1

### Step e

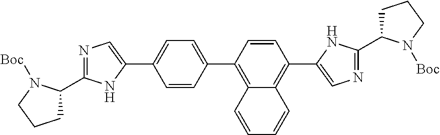

To a mixture of (S)-tert-butyl 2-(5-(4-bromonaphthalen-1-yl)-1H-imidazol-2-yl)pyrrolidine-1-carboxylate (280 mg, 0.63 mmol) and (5)-tert-butyl 2-(5-(4-(4,4,5,5-tetramethyl-1,3,2-dioxaborolan-2-yl)phenyl)-1H-imidazol-2-yl)pyrrolidine-1-carboxylate (see patent application WO 2008/021927; 278 mg, 0.56 mmol) in dioxane:water (10 mL:2 mL) was added K₂CO₃ (175 mg, 1.26 mmol). The reaction mixture was purged with N₂ for 10 min. Then Pd(dppf)Cl₂ (26 mg, 0.031 mmol) was added and purged with N₂ for further 10 min. The reaction mixture was heated at 90° C. overnight. The volatiles were evaporated and the residue was suspended in DCM, filtered through a short pad of diatomaceous earth (Celite®). Then the organic layer was separated, washed with water, brine, dried over Na₂SO₄ and concentrated in vacuo. The crude was purified by flash chromatography (ISCO, MeOH: CHCl₃, 5:95) to obtain phenyl-napthalene 1e (260 mg). LC/MS (Condition 8): Rₜ=1.65 min. ¹H NMR (DMSO-d₆, δ=2.50 ppm, 400 MHz): δ 12.11/11.97/11.87 (br s, 2H), 8.87 (br s, 1H), 7.92-7.83 (m, 3H), 7.73-7.45 (m, 8H), 4.97-4.81 (m, 2H), 3.62-3.52 (m, 2H), 3.42-3.34 (m, 2H), 2.33-1.82 (m, 8H), 1.44-1.20 (m, 18H). LC/MS: Anal. Calcd. for [M+H]⁺ C₄₀H₄₇N₆O₄: 675.36; found 675.2.

### Example 1

### Step f

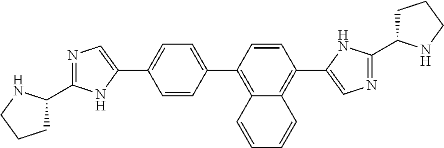

To a solution of carbamate 1e (130 mg, 0.19 mmol) in MeOH (30 mL) was added HCl/ether (20 mL) and stirred at room temperature for 1 h. The volatile component was removed in vacuo, and the residue was washed with ether. The resulting salt was exposed to high vacuum to afford the HCl salt of pyrrolidine if as a solid (85 mg), which was submitted to the next step as such. LC/MS (Condition 8): Rₜ=1.18 min. ¹H NMR (DMSO-d₆, δ=2.50 ppm, 400 MHz): δ 10.41/10.32 (br s, 2H), 8.42-8.37 (m, 1H), 8.17 (br s, 1H), 8.10 (app d, 2H), 7.99 (br s, 1H), 7.95 (app d, 1H), 7.82 (app d, 1H), 7.73-7.58 (m, 5H), 5.06 (br s, 2H), 3.43-3.36 (m, 4H), 2.50-1.98 (m, 8H). LC/MS: Anal. Calcd. for [M+H]⁺ C₃₀H₃₁N₆: 475.25; found 475.2.

### Example 1

To a solution of pyrrolidine 1f/4HCl (80 mg, 0.16 mmol) in DMF (15 mL) was added DIEA (0.23 mL, 1.34 mmol) at room temperature. Then (S)-2-(methoxycarbonylamino)-3-methylbutanoic acid (62 mg, 0.35 mmol) was added followed by HATU (131 mg, 0.34 mmol). After being stirred for 2 hrs at room temperature, the volatile component was removed in vacuo and the residue was extracted with DCM, washed with water, dried over Na₂SO₄ and concentrated in vacuo. The crude was submitted to a reverse phase HPLC purification (ACN/water/TFA) to afford the TFA salt of Example 1 (55 mg) as a solid. LC (Condition 1 and 2): >98% homogeneity index. LC/MS (Condition 8): Rₜ=1.48 min. ¹H NMR (MeOD, δ=3.34 ppm, 400 MHz): δ 8.04-7.92 (m, 5H), 7.81-7.75 (m, 2H), 7.70-7.60 (m, 5H), 5.34-5.28 (m, 2H), 4.28-4.26 (m, 2H), 4.18-4.10 (m, 2H), 3.92-3.87 (m, 2H), 3.68 (s, 6H), 2.65-2.58 (m, 2H), 2.35-2.07 (m, 8H), 1.05-0.93 (m, 12H). LC/MS: Anal. Calcd. for [M+H]⁺ C₄₄H₅₃N₈O₆: 789.4; found 789.4.

### Example 2

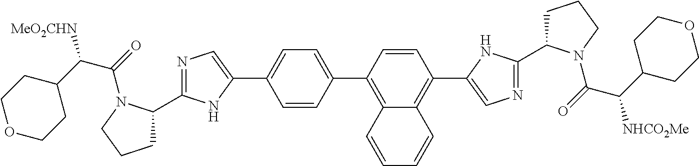

The TFA salt of Example 2 was prepared from pyrrolidine 1f/4HCl and (S)-2-(methoxycarbonylamino)-2-(tetrahydro-2H-pyran-4-yl)acetic acid according to the procedure described for the synthesis of Example 1. LC (Condition 1 and 2): >95% homogeneity index. LC/MS (Condition 8): Rₜ=1.36 min. LC/MS: Anal. Calcd. for [M+H]⁺₄₈H₅₇N₈O₈: 873.42; found 873.4.

### Example 3

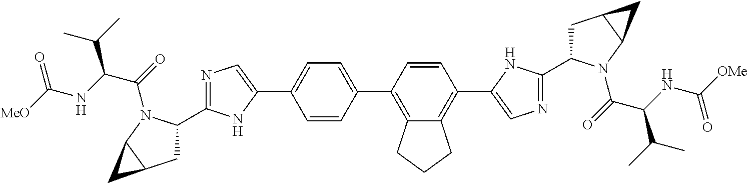

### Example 3

### Step a

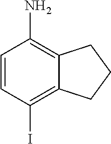

To a solution of 4-aminoindan (5 g, 37.56 mmol) in acetic acid (350 mL) was added dropwise ICl (6.09 g, 37.56 mmol) in acetic acid (14 mL) at room temperature. The resulting solution was stirred overnight. Upon completion of the reaction, the solvent was evaporated under reduced pressure. The residue was dissolved in EtOAc (200 mL) and the solution was washed with saturated NaHCO₃ solution and the aqueous layer was extracted with EtOAc (200 mL). The combined extracts were washed with water, brine, dried over Na₂SO₄ and concentrated in vacuo. The crude was purified by flash chromatography (ISCO, EtOAc: petroleum ether, 11:89) to obtain iodide 3a (6.4 g) as a white solid. LC/MS (Condition 9): Rₜ=1.40 min. ¹H NMR (CDCl₃, δ=7.26 ppm, 400 MHz): δ 7.31 (d, J=8.4, 1H), 6.29 (d, J=8.4, 1H), 3.55 (br s, 2H), 2.91-2.84 (m, 4H), 2.13-2.06 (m, 2H). LC/MS: Anal. Calcd. for [M+H]⁺ C₉H₁₁IN: 259.99; found 260.0.

### Example 3

### Step b

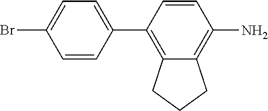

To a stirred solution of iodide 3a (6.2 g, 23.9 mmol), 4-bromophenylboronicacid (5.28 g, 26.3 mmol) in anhydrous MeOH (100 mL), K₂CO₃ (7.43 g, 53.7 mmol) was added and reaction mixture was purged with N₂ for 10 minutes. Then Pd(Ph₃P)₄ (828 mg, 0.71 mmol) was added and reaction mixture was purged with N₂ for further 10 minutes and heated at 60° C. for overnight. Upon completion of the reaction, the mixture was filtered through a pad of diatomaceous earth (Celite®) and concentrated under reduced pressure. The residue was dissolved in EtOAc (250 mL), washed with water and the aqueous layer was extracted with EtOAc (250 mL). The combined extracts were washed with brine, dried over Na₂SO₄ and concentrated in vacuo. The crude was purified by flash chromatography (ISCO, EtOAc: petroleum ether, 12:88) to obtain bromide 3b (4 g) as an off-white solid. LC/MS (Condition 9): Rₜ=1.75 min. ¹H NMR (CDCl₃, δ=7.26 ppm, 400 MHz): δ 7.50-7.47 (m, 2H), 7.28-7.25 (m, 2H), 7.03 (d, J=8.0, 1H), 6.60 (d, J=8.0, 1H), 3.65 (br s, 2H), 2.95 (t, J=7.4, 2H), 2.78 (t, J=7.2, 2H), 2.13-2.06 (m, 2H). LC/MS: Anal. Calcd. for [M+H]⁺ C₁₅H₁₅BrN: 288.03; found 288.0.

### Example 3

### Step c

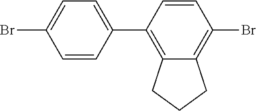

A solution of bromide 3b (4 g, 13.9 mmol) in 48% aqueous HBr (20 mL) was cooled to −15° C. Then an ice-cold solution of NaNO₂ (1.92 g, 27.8 mmol) in water (15 mL) was added slowly and the temperature was maintained between −10° C. to −15° C. for 30 minutes. CuBr (200 mg, 1.39 mmol) was added to the above reaction mixture at −15° C., maintained below 15° C. for 3 hrs and slowly allowed to warm to room temperature over 12 hrs. The pH of the reaction mixture was adjusted to ˜10 by addition of 10% NaOH. The reaction mixture was filtered through diatomaceous earth (Celite®) and washed with EtOAc. The layers were separated and the aqueous layer was extracted twice with EtOAc. The combined extracts were washed with brine, dried over Na₂SO₄ and concentrated in vacuo. The crude was purified by flash chromatography (SiO₂, 230-400 mesh, petroleum ether) to obtain dibromide 3c (2.1 g) as a white solid. ¹H NMR (CDCl₃, δ=7.26 ppm, 400 MHz): δ 7.55-7.53 (m, 2H), 7.38 (d, J=8.0, 1H), 7.27-7.25 (m, 2H), 7.0 (d, J=8.0, 1H), 3.02 (t, J=7.2, 2H), 3.01 (t, J=7.6, 2H), 2.10-2.03 (m, 2H).

### Example 3

### Step d

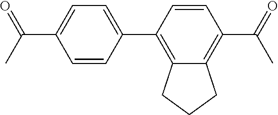

To a stirred solution of dibromide 3c (1.4 g, 4.0 mmol) in anhydrous dioxane (20 mL) was added tributyl(1-ethoxyvinyl)tin (5.7 g, 16 mmol), and the reaction mixture was purged with N₂ for 30 minutes. Then Pd(Ph₃P)₂Cl₂ (280 mg, 0.4 mmol) was added, purged with N₂ for 10 minutes and the mixture was stirred at 90° C. for 16 hrs. The mixture was filtered through a pad of diatomaceous earth (Celite®) and the filtrate was concentrated under reduced pressure. The resulting residue was dissolved in EtOAc and cooled to ice bath temperature. Then 2N HCl (20 mL) was added slowly and allowed to warm to room temperature over a period of 2 hrs. The organic layer was separated and the aqueous phase was back extracted with EtOAc (150 mL). The combined extracts were washed with 10% NaHCO₃, water, brine, dried over Na₂SO₄ and concentrated in vacuo. The crude was purified by flash chromatography (ISCO, EtOAc: petroleum ether, 10:90) to obtain diketone 3d (700 mg) as a pale yellow solid. LC/MS (Condition 9): Rₜ=2.17 min. ¹H NMR (CDCl₃, δ=7.26 ppm, 400 MHz): δ 8.06-8.04 (m, 2H), 7.78 (d, J=8.0, 1H), 7.56-7.54 (m, 2H), 7.30 (d, J=8.0, 1H), 3.34 (t, J=7.6, 2H), 2.95 (t, J=7.4, 2H), 2.66 (s, 3H), 2.64 (s, 3H), 2.12-2.04 (m, 2H). LC/MS: Anal. Calcd. for [M+H]⁺ C₁₉H₁₉O₂: 279.13; found 279.2.

### Example 3

### Step e

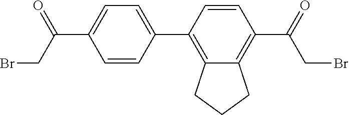

To a solution of diketone 3d (1.03 g, 3.7 mmol) in anhydrous dioxane (20 mL) was added Br₂ (1.18 g, 7.4 mmol) in dioxane (5 mL) at 0° C. and the mixture was allowed to warm to room temperature over a period of 3 hrs. Upon completion of the reaction, the solvent was evaporated under reduced pressure. The residue was dissolved in DCM (100 mL) and the solution was washed with saturated NaHCO₃ solution and the aqueous phase was extracted with DCM (100 mL). The combined extracts were dried over Na₂SO₄ to obtain dibromide 3e (1.5 g) which was used as such in the next step. LC/MS (Condition 9): Rₜ=2.12 min. ¹H NMR (CDCl₃, δ=7.26 ppm, 400 MHz): δ 8.09-8.03 (m, 2H), 7.77 (d, J=8.0, 1H), 7.58-7.53 (m, 2H), 7.31 (d, J=8.0, 1H), 4.48 (s, 4H), 3.35-3.32 (m, 2H), 2.96-2.94 (m, 2H), 2.11-2.07 (m, 2H). LC/MS: Anal. Calcd. for [M+H]⁺ C₁₉H₁₂Br₂O₂: 434.95; found 435.0.

### Example 3

### Step f

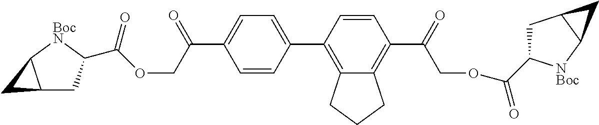

(1R,3S,5R)-2-(tert-butoxycarbonyl)-2-azabicyclo[3.1.0]hexane-3-carboxylic acid (1.57 g, 6.92 mmol) in acetonitrile (50 mL) was cooled to ice bath temperature and DIEA (1.11 g, 8.6 mmol) was added. Dibromide 3e (1.5 g, 3.46 mmol) dissolved in acetonitrile (50 mL) was added slowly to the above reaction mixture and allowed to warm to room temperature over a period of 3 hrs. Upon completion of the reaction, the solvent was evaporated under reduced pressure. The residue was dissolved in EtOAc (200 mL) and the solution was washed with saturated NaHCO₃ solution, saturated NH₄Cl and the aqueous phase was extracted with EtOAc (100 mL). The combined extracts were washed with water, brine, dried over Na₂SO₄ and concentrated in vacuo. The crude was purified by flash chromatography (ISCO, EtOAc: petroleum ether, 25:75) to obtain diketoester 3f (1.2 g) as a white solid. LC/MS (Condition 10): Rₜ=2.47 min. ¹H NMR (CDCl₃, δ=7.26 ppm, 400 MHz): δ 7.99 (d, J=8.0, 2H), 7.69 (d, J=8.0, 1H), 7.56 (d, J=8.0, 2H), 7.29 (d, J=8.0, 1H), 5.56-5.12 (m, 4H), 4.23 (br s, 2H), 3.55 (br s, 1H), 3.47 (br s, 1H), 3.31 (app t, 2H), 2.94 (app t, 2H), 2.66-2.59 (m, 2H), 2.51-2.45 (m, 2H), 2.12-2.06 (m, 2H), 1.73-1.64 (m, 2H), 1.47 (br s, 18H), 0.90-0.85 (m, 2H), 0.53-0.47 (m, 2H). LC/MS: Anal. Calcd. for [M+H₂O]⁺ C₄₁H₅₀N₂O₁₁: 746.34; found 746.4.

### Example 3

### Step g-1 & g-2

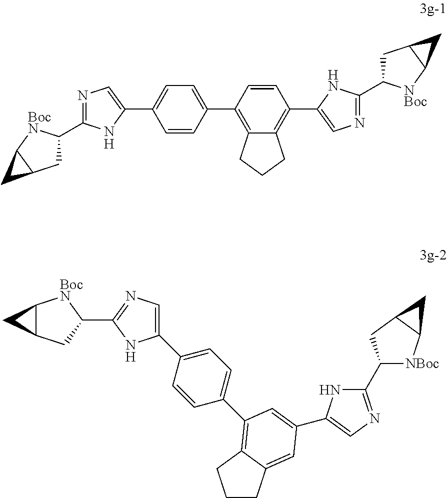

A solution of diketoester 3f (700 mg, 0.961 mmol) in anhydrous xylenes (50 mL) was added anhydrous NH₄OAc (1.480 g, 19.23 mmol). The reaction mixture was purged with N₂ for 10 minutes and heated at 130° C. for overnight. The solvent was evaporated under reduced pressure and the residue was dissolved in EtOAc (200 mL), washed with saturated NaHCO₃ solution and the aqueous phase was extracted with EtOAc (100 mL). The combined extracts were washed with water, brine, dried over Na₂SO₄ and concentrated in vacuo. A 500 mg of another batch was performed by using the same above conditions. The crude of both batches were pooled and was submitted to reverse phase HPLC purification (ACN/water/NH₄OAc) to obtain imidazole 3g-1 (495 mg) as a white solid. LC/MS (Condition 11): Rₜ=2.08 min. ¹H NMR (MeOD, δ=3.34 ppm, 400 MHz): δ 7.77 (d, J=8.0, 2H), 7.60 (app br d, 1H), 7.49 (d, J=8.4, 2H), 7.37 (s, 1H), 7.27 (d, J=8.0, 1H), 7.18 (s, 1H), 4.70 (br s, 2H), 3.61 (br s, 2H), 3.12 (t, J=7.2, 2H), 3.07 (app t, 2H), 2.58-2.52 (m, 2H), 2.39-2.34 (m, 2H), 2.15-2.10 (m, 2H), 1.78-1.71 (m, 2H), 1.31-1.28 (br s, 18H), 0.90-0.87 (m, 2H), 0.63 (br s, 2H). LC/MS: Anal. Calcd. for [M+H]⁺ C₄₁H₄₉N₆O₄: 689.37; found 689.3. The regioisomer 3g-2 (62 mg) was also isolated from the same reaction (eluted later) as a white solid. LC/MS (Condition 11): Rₜ=2.10 min. ¹H NMR (DMSO-d₆, δ=2.50 ppm, 400 MHz): δ 11.89 (s, 1H), 11.81 (s, 1H), 7.85-7.72 (m, 2H), 7.62-7.58 (m, 2H), 7.52-7.45 (m, 4H), 4.65-4.62 (m, 2H), 3.48-3.39 (m, 2H), 2.96-2.93 (m, 4H), 2.40-2.22 (m, 4H), 2.03-1.98 (m, 2H), 1.67-1.60 (m, 2H), 1.35-1.15 (br s, 18H), 0.80-0.71 (m, 2H), 0.58-0.51 (m, 2H). LC/MS: Anal. Calcd. for [M+H]⁺ C₄₁H₄₉N₆O₄: 689.37; found 689.4.

### Example 3

### Step h

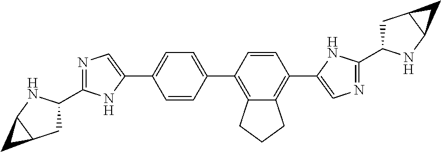

To a solution of carbamate 3g-1 (11 mg, 0.015 mmol), in MeOH (1.0 mL) was added HCl/dioxane (4N; 3 mL) at 0° C. and the mixture was stirred at room temperature for 3 hrs. The volatile component was removed in vacuo, the residue was co-evaporated with anhydrous DCM (3×5 mL) and dried under high vacuum to afford pyrrolidine 3h (4HCl) as a pale yellow solid (10 mg). LC/MS (Condition 10): Rₜ=1.49 min. ¹H NMR (DMSO-d₆, δ=2.50 ppm, 400 MHz): δ 10.24 (br s, 2H), 7.88 (d, J=8.0, 2H), 7.83 (s, 1H), 7.77 (app d, 1H), 7.56-7.54 (m, 3H), 7.32 (d, J=8.0, 1H), 4.70-4.62 (m, 2H), 3.40-3.35 (obscured, 2H), 3.12 (t, J=7.2, 2H), 3.03 (app t, 2H), 2.08-2.01 (m, 2H), 1.93 (br s, 2H), 1.30-1.25 (m, 2H), 1.14 (br s, 2H), 0.90-0.79 (m, 4H). LC/MS: Anal. Calcd. for [M+H]⁺ C₃₁H₃₃N₆: 489.27; found 489.3.

### Example 3

HATU (12.4 mg, 0.032 mmol) was added to the DMF solution of pyrrolidine 3h (4HCl) (9.5 mg, 0.015 mmol), (S)-2-(methoxycarbonylamino)-3-methylbutanoic acid (5.87 mg, 0.033 mmol) and DIEA (0.022 mL, 0.12 mmol) and stirred at room temperature for 2 hrs. The volatile component was removed in vacuo, the residue was dissolved in EtOAc (100 mL), washed with saturated NaHCO₃ solution, saturated NH₄Cl and the aqueous phase was extracted with EtOAc (50 mL). The combined extracts were washed with water, brine, dried over Na₂SO₄ and concentrated in vacuo. The crude was submitted to reverse phase HPLC purification (ACN/water/NH₄OAc) to afford Example 3 as a free base (2 mg, pale-yellow solid). LC (Condition 1 and 2): >98% homogeneity index. LC/MS (Condition 11): Rₜ=1.78 min. ¹H NMR (DMSO-d₆, δ=2.50 ppm, 400 MHz): δ 12.09/11.90/11.72/11.69 (br s, 2H), 7.82-7.66 (m, 3H), 7.53-7.43 (m, 3H), 7.30-7.21 (m, 2H), 7.16-7.06 (m, 2H), 5.15-5.10 (m, 2H), 4.45 (t, J=7.2, 2H), 3.56 (br s, 8H), 3.12-3.06 (m, 2H), 3.04-2.99 (m, 2H), 2.50-2.46 (m, 2H), 2.26-2.20 (m, 2H), 2.12-2.01 (m, 4H), 1.89-1.86 (m, 2H), 1.06-0.87 (m, 14H), 0.73 (br s, 2H). LC/MS: Anal. Calcd. for [M−H]⁻ C₄₅H₅₃N₈O₆: 801.42; found 801.4.

### Example 4

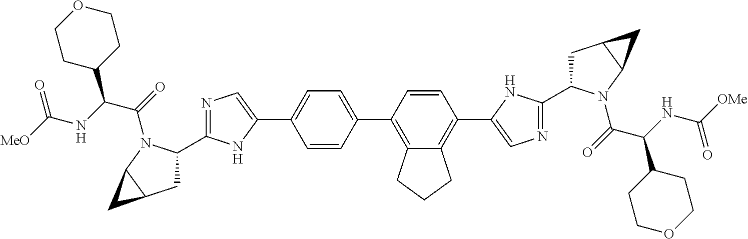

Example 4 (free base with residual ammonium acetate) was prepared from pyrrolidine 3h (HCl salt) and (S)-2-(methoxycarbonylamino)-2-(tetrahydro-2H-pyran-4-yl)acetic acid according to the procedure described for Example 3. LC (Condition 1 and 4): >98% homogeneity index. LC/MS (Condition 10): Rₜ=1.51 min. LC/MS: Anal. Calcd. for [M+H]⁺ C₄₉H₅₉N₈O₈: 887.44; found 887.4.

### Example 5

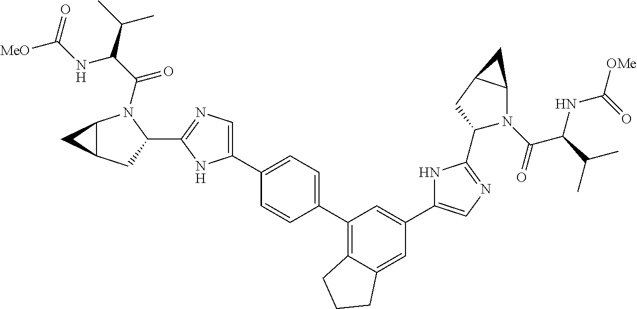

Example 5 (free base with residual ammonium acetate) was prepared from carbamate 3g-2 according to the procedure described for the preparation of its regioisomer Example 3. LC (Condition 1 and 2): >97% homogeneity index. LC/MS (Condition 10): Rₜ=1.84 min. ¹H NMR (MeOD, δ=3.34 ppm, 400 MHz): δ 7.76 (d, J=8.0, 2H), 7.54-7.51 (m, 4H), 7.35 (s, 1H), 7.28 (s, 1H), 5.18-5.16 (m, 2H), 4.62-4.57 (m, 2H), 3.72-3.63 (m, 8H), 3.05-2.98 (m, 4H), 2.58-2.49 (m, 2H), 2.48-2.39 (m, 2H), 2.20-2.10 (m, 6H), 1.17-1.10 (m, 2H), 1.04-0.93 (m, 12H), 0.82-0.77 (m, 2H). LC/MS: Anal. Calcd. for [M+H]⁺ C₄₅H₅₅N₈O₆: 803.42; found 803.4.

### Example 6

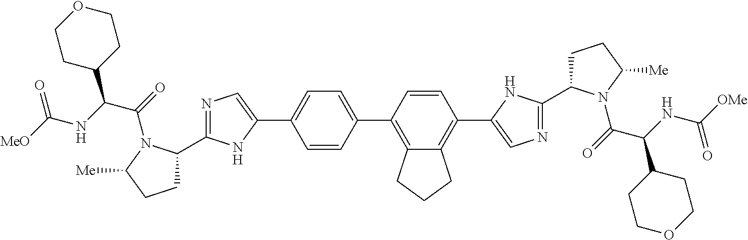

### Example 6

### Step a

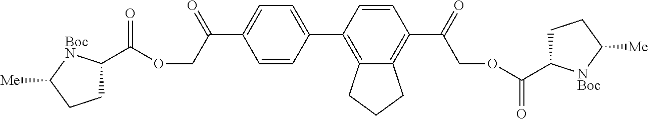

Diketoester 6a was prepared from dibromide 3e and (2S,5S)-1-tert-butyl 2-methyl 5-methylpyrrolidine-1,2-dicarboxylate according to the procedure described for the preparation of diketoester 3f.

### Example 6

### Step b-1 & b-2

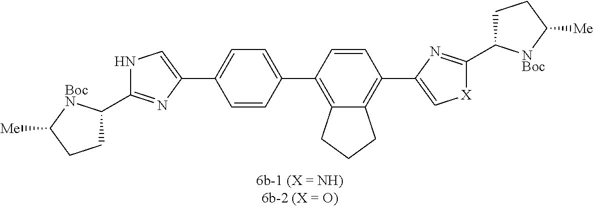

A solution of diketoester 6a (700 mg, 0.956 mmol) in anhydrous xylenes (15 mL) was added anhydrous NH₄OAc (1.47 g, 19.12 mmol). The reaction mixture was purged with N₂ for 10 minutes and heated at 130° C. for overnight. The solvent was evaporated under reduced pressure and the residue was dissolved in EtOAc (200 mL), washed with saturated NaHCO₃ solution and the aqueous phase was extracted with EtOAc (100 mL). The combined extracts were washed with water, brine, dried over Na₂SO₄ and concentrated in vacuo. The crude was submitted to reverse phase HPLC purification (ACN/water/NH₄OAc) to obtain imidazole 6b-1 (115 mg) as a white solid. The oxazoimidazole 6b-2 (103 mg) was also isolated from the same reaction (eluted later) as a white solid.

### Example 6

Example 6 (TFA salt) was prepared starting from bis-imidazole 6b-1 according to the procedure described for the preparation of Example 3 and Example 4. LC (Condition 5 and 6): >96% homogeneity index. LC/MS (Condition 10): Rₜ=1.70 min. ¹H NMR (MeOD, δ=3.34 ppm, 400 MHz): δ 7.98-7.96 (m, 0.5H), 7.89 (s, 0.8H), 7.85 (d, J=8.4, 1.7H), 7.73-7.66 (m, 3.7H), 7.54 (d, J=8.0, 0.9H), 7.44-7.41 (m, 1.4H), 5.75-5.71 (m, 0.3H), 5.22-5.15 (m, 1.7H), 4.24-4.22 (m, 2H), 4.01-3.90 (m, 4H), 3.72/3.69/3.67 (s, 6H), 3.45-3.23 (obscured, 6H), 3.18-3.07 (m, 4H), 2.58-1.72 (m, 12H), 1.60-1.25 (m, 14H). LC/MS: Anal. Calcd. for [M+H]⁺ C₄₉H₆₃N₈O₈: 891.47; found 891.4.

### Example 7

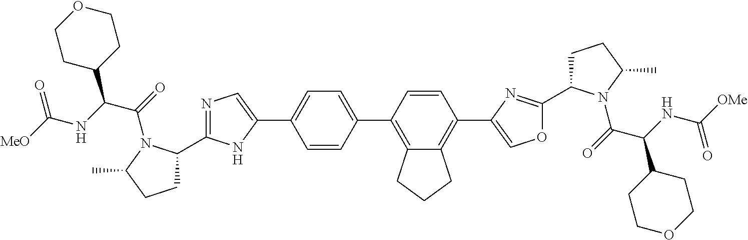

Example 7 (TFA salt) was prepared starting from oxazole 6b-2 according to the procedure described for the preparation of Example 3 and Example 4. LC (Condition 1 and 2): >97% homogeneity index. LC/MS (Condition 10): Rₜ=1.90 min. ¹H NMR (MeOD, δ=3.34 ppm, 400 MHz): δ 8.06 (s, 0.8H), 7.97/7.94/7.92 (s, 0.6H), 7.88 (s, 0.8H), 7.82-7.77 (m, 2.8H), 7.65 (d, J=8.4, 2H), 7.30 (d, J=8.0, 1H), 5.82/5.4/5.20-5.13 (m, 2H), 4.82-4.21 (m, 2H), 4.0-3.87 (m, 4H), 3.72/3.68 (s, 6H), 3.42-3.25 (obscured, 5H), 3.18-3.02 (m, 5H), 2.58-2.11 (m, 8H), 2.02-1.71 (m, 6H), 1.68-1.19 (m, 12H). LC/MS: Anal. Calcd. for [M−H]⁻ C₄₉H₆₀N₇O₉: 890.45; found 890.4.

### Example 7.1

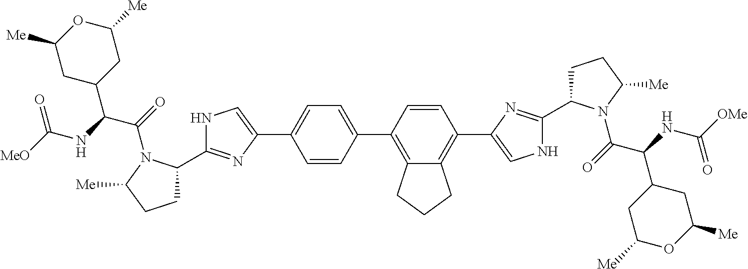

Example 7.1 (TFA salt) was prepared starting from bis-imidazole 6b-1 and appropriate acid according to the procedure described for the preparation of Example 6. LC (Condition 1 and 2): >94% homogeneity index. LC/MS (Condition 10): Rₜ=1.87 min. LC/MS: Anal. Calcd. for [M−H]⁻ C₅₃H₆₉N₈O₈: 945.53; found 945.4.

### Example 8

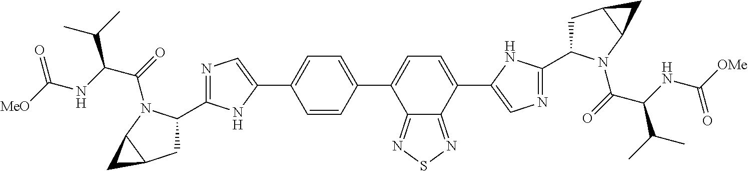

### Example 8

### Step a

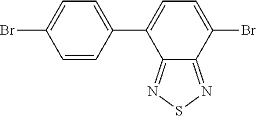

A solution of 4,7-dibromobenzo[c][1,2,5]thiadiazole (1.0 g, 3.4 mmol), 4-bromophenylboronic acid (0.68 g, 3.4 mmol), and K₂CO₃ (0.939 g, 6.8 mmol) in MeOH (50 mL) was purged with N₂ for 10 min. Then Pd(Ph₃P)₄ (0.117 g, 0.10 mmol) was added, the reaction mixture was purged with N₂ for further 10 min and refluxed at 60° C. for over night. The volatile components were evaporated under reduced pressure and H₂O was added to the resulting residue. The crude was extracted with EtOAC (100 mL), and the organic layer was washed with brine, dried over Na₂SO₄ and concentrated in vacuo. The crude was purified by flash chromatography (ISCO, EtOAc: petroleum ether, 2:98) to obtain dibromide 8a (630 mg) as a yellow solid. ¹H NMR (CDCl₃, δ=7.26 ppm, 400 MHz): δ 7.94 (d, J=7.4, 1H), 7.81-7.78 (m, 2H), 7.69-7.66 (m, 2H), 7.58 (d, J=7.4, 1H).

### Example 8

### Step b

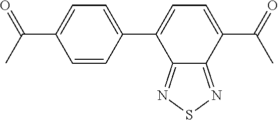

A solution dibromide 8a (1.0 g, 2.7 mmol) in dioxane (20 mL) was purged with N₂ for 10 min. Then tributyl(1-ethoxyvinyl)tin (3.75 mL, 10.8 mmol) was added followed by Pd(Ph₃P)₂Cl₂ (0.135 g, 0.192 mmol). The reaction mixture was purged with N₂ for further 10 min and heated at 80° C. for 1 h under microwave conditions. The reaction mixture was filtered through a diatomaceous earth (Celite®) plug and volatile components were evaporated under reduced pressure. The resulting residue was dissolved in EtOAc (30 mL) and HCl (1.5 N, 50 mL) was added at room temperature. After being stirred for 2 h, the reaction mixture was neutralized with NaHCO₃, extracted with EtOAc (100 mL), and the organic layer was washed with brine, dried over Na₂SO₄ and concentrated in vacuo. The crude was purified by flash chromatography (ISCO, EtOAc: petroleum ether, 20:80) to obtain diketone 8b (630 mg) as a yellow solid. LC/MS (Condition 9): Rₜ=1.90 min. ¹H NMR (CDCl₃, δ=7.26 ppm, 400 MHz): δ 8.40 (d, J=7.4, 1H), 8.15 (d, J=8.4, 2H), 8.07 (d, J=8.4, 2H), 7.88 (d, J=7.4, 1H), 3.08 (s, 3H), 2.69 (s, 3H). LC/MS: Anal. Calcd. for [M+H]⁺ C₁₆H₁₃N₂O₂S: 297.06; found 297.0.

### Example 8

### Step c

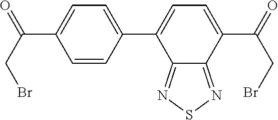

To a stirred solution of diketone 8b (0.480 g, 1.62 mmol) in CHCl₃ (10 mL), Br₂ (0.77 g, 4.83 mmol) in CHCl₃ (10 mL) was added at room temperature and heated to 60° C. for 3 h. The reaction mixture was neutralized with NaHCO₃, extracted with CH₂Cl₂ (100 mL), dried over Na₂SO₄ and concentrated in vacuo to obtain crude dibromide 8c (760 mg) which was submitted to the next step as such. LC/MS (Condition 9): Rₜ=2.09 min. LC/MS: Anal. Calcd. for [M+H]⁺ C₁₆H₁₁Br₂N₂O₂S: 452.88; found 452.8.

### Example 8

### Step d

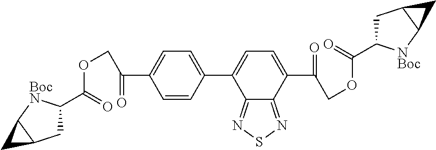

To a stirred solution of (1R,3S,5R)-2-(tert-butoxycarbonyl)-2-azabicyclo[3.1.0]hexane-3-carboxylic acid (1.51 g, 6.69 mmol) and Et₃N (0.93 mL, 6.69 mmol) in CH₃CN (20 mL) was added dibromide 8c (0.760 g, 1.67 mmol) in CH₃CN (15 mL) at room temperature. The reaction mixture was stirred for 90 min and then volatiles were evaporated under reduced pressure. Water was added to the reaction mixture, extracted with DCM (100 mL), and the organic layer was washed with brine, dried over Na₂SO₄ and concentrated in vacuo. The crude was purified by flash chromatography (ISCO, EtOAc: petroleum ether, 40:80) to afford diketoester 8d (430 mg) as a yellow solid. LC/MS (Condition 12): Rₜ=1.99 min. ¹H NMR (MeOD, δ=3.34 ppm, 400 MHz): δ 8.54 (d, J=7.6, 1H), 8.29-8.24 (m, 2H), 8.21-8.16 (m, 2H), 8.11 (d, J=7.6, 1H), 6.07/6.02 (br s, 1H), 5.93/5.89 (br s, 1H), 5.68/5.65 (br s, 1H), 5.58-5.47 (m, 1H), 4.35-4.30 (m, 2H), 3.51-3.48 (m, 2H), 2.71-2.62 (m, 2H), 2.59-2.48 (m, 2H), 1.79-1.71 (m, 2H), 1.50 (br s, 18H), 0.93-0.87 (m, 2H), 0.58-0.57 (m, 2H). LC/MS: Anal. Calcd. for [M−H]⁻ C₃₈H₄₁N₄O₁₀S: 745.26; found 745.8.

### Example 8

### Step e

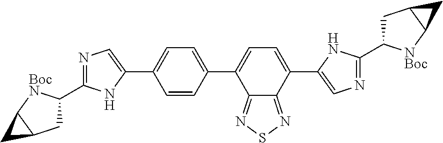

To a solution of diketoester 8d (0.4 g, 0.536 mmol) in xylenes (8 mL) was added NH₄OAc (0.826 g, 10.72 mmol) and heated at 130° C. for overnight in a sealed tube. The volatile components were evaporated under reduced pressure and the reaction mixture was treated with saturated NaHCO₃ solution. Then the reaction mixture was extracted with CH₂Cl₂ (100 mL), and the organic layer was washed with water, brine, dried over Na₂SO₄ and concentrated in vacuo. The crude was purified by flash chromatography (ISCO, MeOH: DCM: 0.3:99.7) to obtain imidazole 8e (100 mg) as a orange-red solid. LC/MS (Condition 12): Rₜ=1.71 min. ¹H NMR (DMSO-d_{6, δ=2.50} ppm, 400 MHz): δ 12.16 (br s, 1H), 11.94 (br s, 1H), 8.31 (d, J=7.6, 1H), 8.16/8.15 (s, 1H), 8.02 (d, J=8.4, 2H), 7.94 (d, J=7.6, 1H), 7.91 (d, J=8.4, 2H), 7.59 (br s, 1H), 4.75-4.62 (m, 2H), 3.50-3.40 (m, 2H), 2.48-2.25 (m, 4H), 1.70-1.62 (m, 2H), 1.46-1.14 (br s, 18H), 0.81-0.73 (m, 2H), 0.62-0.53 (m, 2H). LC/MS: Anal. Calcd. for [M−H]⁻ C₃₈H₄₁N₈O₄S: 705.3; found 704.8.

### Example 8

### Step f

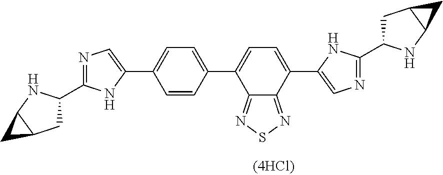

To a solution of carbamate 8e (0.105 g, 0.148 mmol) in MeOH (6 mL) was added HCl/dioxane (4N, 6.3 mL) and stirred at room temperature for 2 h. The volatile component was removed in vacuo, and the residue was co-evaporated with dry CH₂Cl₂ (3×5 mL). The resulting salt was exposed to high vacuum to afford pyrrolidine 8f (105 mg) as a yellow solid which was submitted to the next step as such. LC/MS (Condition 9): Rₜ=1.33 min. ¹H NMR (MeOD, δ=3.34 ppm, 400 MHz): δ 8.71 (s, 1H), 8.48 (d, J=7.6, 1H), 8.31 (d, J=8.4, 2H), 8.21 (s, 1H), 8.13 (d, J=7.6, 1H), 8.07 (d, J=8.4, 2H), 5.18-5.02 (m, 2H), 3.40-3.30 (obscured, 2H), 2.95-2.81 (m, 4H), 2.23-2.18 (m, 2H), 1.38-1.33 (m, 2H), 1.15-1.11 (m, 2H). LC/MS: Anal. Calcd. for [M+H]⁺ C₂₈H₂₇N₈S: 507.2; found 507.2.

### Example 8

To a solution of HCl salt of pyrrolidine 8f (0.055 g, 0.077 mmol) in DMF (2 mL) was added (S)-2-(methoxycarbonylamino)-3-methylbutanoic acid (0.0286 g, 0.163 mmol), HATU (0.0607 g, 0.159 mmol) followed by DIEA (0.10 mL, 0.622 mmol) at 0° C. After being stirred for 2 h at room temperature, the volatile component was removed in vacuo and the residue was dissolved in DCM (50 mL), washed with saturated solution of NH₄Cl, NaHCO₃ solution, brine, dried over Na₂SO₄ and concentrated in vacuo. The crude was submitted to reverse phase HPLC purification (ACN/water/TFA) to afford TFA salt of Example 8 (47 mg) as a yellow solid. LC (Condition 1 and 2): >97% homogeneity index. LC/MS (Condition 9): Rₜ=1.56 min. ¹H NMR (MeOD, δ=3.34 ppm, 400 MHz): δ 8.44 (s, 1H), 8.25 (d, J=8.6, 2H), 8.23 (d, J=7.2, 1H), 8.05 (d, J=7.2, 1H), 7.94 (s, 1H), 7.91 (d, J=8.6, 2H), 5.24 (dd, J=9.2, 6.8, 1H), 5.15 (dd, J=9.2, 6.8, 1H), 4.58 (t, J=7.6, 2H), 3.83 (t, J=4.8, 2H), 3.67 (s, 6H), 2.73-2.67 (m, 2H), 2.57-2.48 (m, 2H), 2.23-2.08 (m, 4H), 1.15-1.10 (m, 2H), 1.08-1.02 (m, 6H), 0.97-0.88 (m, 8H). LC/MS: Anal. Calcd. for [M+H]⁺ C₄₂H₄₉N₁₀O₆S: 821.35; found 821.2.

### Example 9

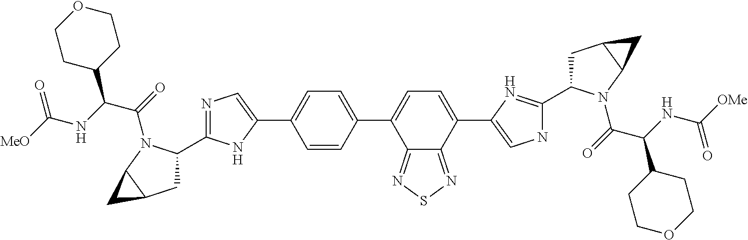

Example 9 (TFA salt; yellow solid) was prepared from pyrrolidine 8f (0.4HCl) and (S)-2-(methoxycarbonylamino)-2-(tetrahydro-2H-pyran-4-yl)acetic acid according to the procedure described for Example 8. LC (Condition 1 and 2): >95% homogeneity index. LC/MS (Condition 9): Rₜ=1.41 min. LC/MS: Anal. Calcd. for [M+H]⁺ C₄₆H₅₃N₁₀O₈S: 905.37; found 905.4.

### Example 10-11

Example-10 & -11 (TFA salt) were prepared starting from dibromide 8c and (2S,5S)-1-tert-butyl 2-methyl 5-methylpyrrolidine-1,2-dicarboxylate according to the procedure described for the preparation Example-8 & -9.

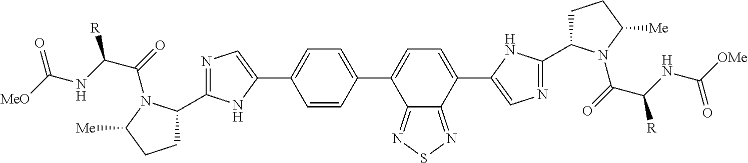

| Example # | R | LC & LC/MS data |
|---|---|---|
| 10 | 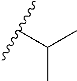 | LC (Condition 1 and 2): >98% homogeneity index. LC/MS (Condition 10): Rₜ = 2.02 min. LC/MS: Anal. Calcd. for [M + H]⁺ C₄₂H₅₃N₁₀O₆S: 825.38; found 825.3. |
| 11 | 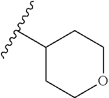 | LC (Condition 1 and 2): >97% homogeneity index. LC/MS (Condition 10): Rₜ = 1.67 min. LC/MS: Anal. Calcd. for [M + H]⁺ C₄₆H₅₇N₁₀O₈S: 909.4; found 909.4. |

### Example 12

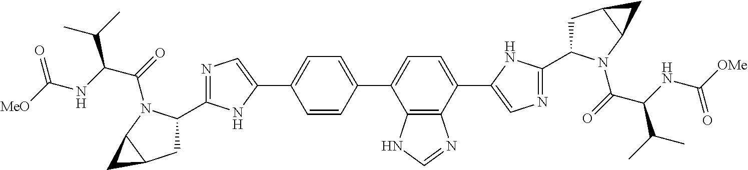

### Example 12

### Step a

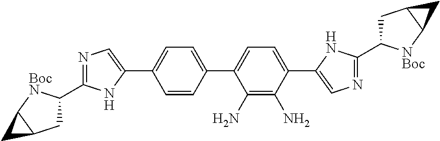

To a solution of 8e (175 mg, 0.247 mmol) in glacial AcOH (5 mL) was added zinc dust (161 mg, 2.47 mmol) and heated at 50° C. for 2 h. The reaction mixture was filtered through a pad of diatomaceous earth (Celite®) and washed with MeOH (2×10mL). The filtrate was evaporated under reduced pressure. The resulting residue was dissolved in EtOAc (50 mL) and washed with saturated NaHCO₃ solution, water, brine, dried over Na₂SO₄ and concentrated in vacuo to obtain crude diamine 12a (120 mg) which was submitted to the next step as such. LC/MS (Condition 12): Rₜ=1.61 min. ¹H NMR (DMSO-d₆, δ=2.50 ppm, 400 MHz): δ 11.92 (br s, 1H), 11.85 (br s, 1H), 7.80 (d, J=8.4, 2H), 7.48 (d, J=1.6, 1H), 7.43/7.37 (d, J=8.0, 2H), 7.33 (d, J=1.6, 1H), 6.91 (d, J=8.0, 1H), 6.40 (d, J=8.0, 1H), 5.96 (br s, 2H), 4.68-4.61 (m, 2H), 4.14-4.11 (m, 2H), 3.47-3.40 (m, 2H), 2.40-2.23 (m, 4H), 1.69-1.60 (m, 2H), 1.30-1.20 (br s, 18H), 0.80-0.72 (m, 2H), 0.60-0.52 (m, 2H). LC/MS: Anal. Calcd. for [M+H]⁺ C₃₈H₄₇N₈O₄: 679.36; found 679.2.

### Example 12

### Step b

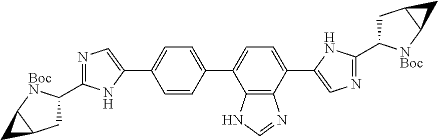

To a solution of diamine 12a (60 mg, 0.088 mmol) in triethyl orthoformate (1 mL) was added catalytic amount of PTSA (5 mg) and heated at 100° C. for 15 minutes under microwave conditions. The reaction mixture was diluted with DCM (50 mL) and washed with saturated NaHCO₃ solution, water, brine, dried over Na₂SO₄ and concentrated in vacuo. The residue was triturated with hexane (5 mL) and the resulting solid was filtered, washed with hexane (3×5 mL) to obtain crude which was submitted to reverse phase HPLC purification (ACN/water/TFA) to afford TFA salt of Example 12b (30 mg) as a white solid. LC/MS (Condition 13): Rₜ=1.77 min. ¹H NMR (MeOD, δ=3.34 ppm, 400 MHz): δ 8.48 (s, 1H), 8.20 (br s, 1H), 8.01-7.91 (m, 5H), 7.80 (d, J=7.6, 1H), 7.58 (d, J=8.0, 1H), 5.01-4.97 (m, 1H), 4.92-4.85 (obscured, 1H), 3.70-3.62 (m, 2H), 2.78-2.69 (m, 2H), 2.52-2.41 (m, 2H), 1.90-1.82 (m, 2H), 1.55-1.25 (br s, 18H), 0.95-0.92 (m, 2H), 0.77-0.72 (m, 2H). LC/MS: Anal. Calcd. for [M−H]⁻ C₃₉H₄₃N₈O₄: 687.35; found 687.5.

### Example 12

### Step c

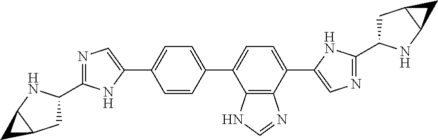

To a solution of carbamate 12b (30 mg, 0.043 mmol) in MeOH (1 mL) was added HCl/dioxane (4N, 1 mL) and stirred at room temperature for 2 h. The volatile component was removed in vacuo, and the residue was co-evaporated with dry CH₂Cl₂ (3×5 mL). The resulting salt was exposed to high vacuum to afford pyrrolidine 12c (22 mg) as a yellow solid which was submitted to the next step as such. LC/MS (Condition 9): Rₜ=1.12 min. ¹H NMR (MeOD, δ=3.34 ppm, 400 MHz): δ 9.53 (s, 1H), 8.20-7.99 (m, 5H), 7.90 (br s, 2H), 7.75 (br s, 1H), 5.05-4.83 (obscured, 2H), 3.73-3.55 (m, 2H), 2.94-2.48 (m, 4H), 2.20-2.06 (m, 2H), 1.18-1.01 (m, 2H), 0.91-0.82 (m, 2H). LC/MS: Anal. Calcd. for [M+H]⁺ C₂₉H₂₉N₈: 489.24; found 489.2.

### Example 12

To a solution of HCl salt of pyrrolidine 12c (22 mg, 0.04 mmol) in DMF (2 mL) was added (S)-2-(methoxycarbonylamino)-3-methylbutanoic acid (16 mg, 0.09 mmol), HATU (33.9 mg, 0.089 mmol) followed by DIEA (0.03 mL, 0.174 mmol) at 0° C. After being stirred for 2 h at room temperature, the volatile component was removed in vacuo and the residue was dissolved in DCM (50 mL), washed with saturated solution of NH₄Cl, NaHCO₃ solution, brine, dried over Na₂SO₄ and concentrated in vacuo. The crude was submitted to reverse phase HPLC purification (ACN/water/TFA) to afford TFA salt of Example 12 (4.2 mg) as a off-white solid. LC (Condition 1 and 7): >91% homogeneity index. LC/MS (Condition 10): Rₜ=1.54 min. LC/MS: Anal. Calcd. for [M+H]⁺ C₄₃H₅₁N₁₀O₆: 803.39; found 803.4.

### Example 13

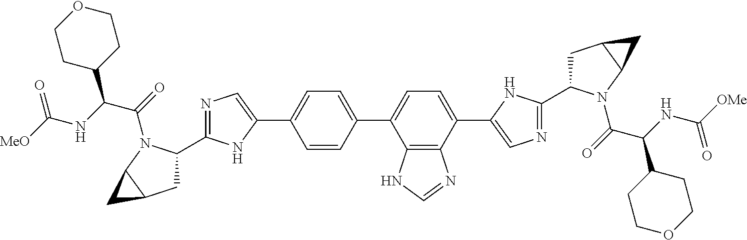

Example 13 (TFA salt; off-white solid) was prepared from pyrrolidine 12c (0.4HCl) and (S)-2-(methoxycarbonylamino)-2-(tetrahydro-2H-pyran-4-yl)acetic acid according to the procedure described for Example 12. LC (Condition 3 and 7): >89% homogeneity index. LC/MS (Condition 10): Rₜ=1.33 min. LC/MS: Anal. Calcd. for [M+H]⁺ C₄₇H₅₅N₁₀O₈: 887.41; found 887.4.

### Example 14

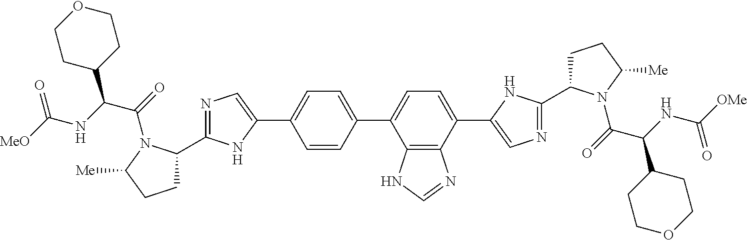

Example-14 (TFA salt) was prepared starting from dibromide 8c and (2S,5S)-1-tert-butyl 2-methyl 5-methylpyrrolidine-1,2-dicarboxylate according to the procedure described for the preparation Example-12 & -13. LC (Condition 1 and 14): >90% homogeneity index. LC/MS (Condition 9): Rₜ=1.40 min. LC/MS: Anal. Calcd. for [M+H]⁺ C₄₇H₅₉N₁₀O₈: 891.44; found 891.4.

### Example 15

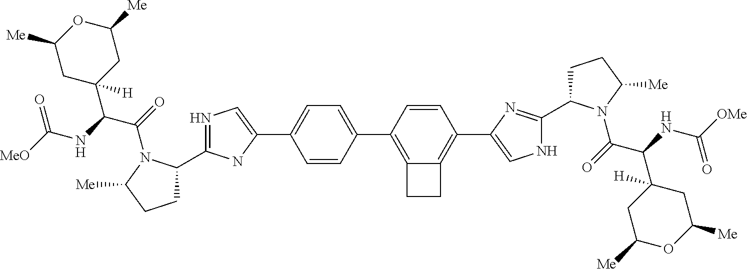

### Example 15

### Step a

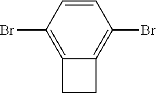

Lithium (0.533 g, 77 mmol) was taken in a 50 mL three necked round-bottom flask, added THF (20 mL) at 0° C. and stirred vigorously. TMSCl (7.36 mL, 57.6 mmol) was added to the reaction mixture followed by drop-wise addition of 1,2-dihydrocyclobutabenzene (2 g, 19.20 mmol). The reaction mixture was stirred at room temperature for 6 days. The reaction mixture was syringed out from the unreacted lithium and then the reaction mixture was quenched with MeOH (10 mL) at 0° C. Water (25 mL) was added and the resulting solution was extracted with petroleum ether (3×40 mL). The combined organic layer was washed with brine (25 mL), dried over Na₂SO₄, and concentrated at 25° C. to yield a mixture of crude dienes (4.6 g) as yellow oil. GC/MS (Condition 16): Rₜ=6.95 min., GC/MS: Anal. Calcd. for [M]⁺ C₁₄H₂₆Si₂: 250.16; found 250.2.

The mixture of crude dienes (7.0 g, 27.9 mmol) was dissolved in THF (50 mL) and heated to 40° C. A solution of DDQ (3.17 g, 13.97 mmol) in THF (20 mL) was added drop-wise to the reaction mixture and heated at the same temperature further for 1 h. Water (100 mL) was added to the reaction mixture and extracted with EtOAc (4×50 mL). The organic layer was washed successively with water (100 mL), saturated Na₂CO₃ (150 mL), brine (50 mL), dried over Na₂SO₄ and concentrated under reduced pressure at 25° C. to yield crude 3,6-bis(trimethylsilyl)-1,2-dihydrocyclobutabenzene (7.9 g). GC/MS (Condition 16): Rₜ=7.60 min. GC/MS: Anal. Calcd. for [M]⁺ C₁₄H₂₄Si₂: 248.14; found 248.0.

A solution of Br₂ (4.66 mL, 91 mmol) in MeOH (20 mL) was added drop-wise to a solution of crude 3,6-bis(trimethylsilyl)-1,2-dihydrocyclobutabenzene (7.5 g, 30.2 mmol) in MeOH (75 mL) at 0° C. and stirred at 25° C. for overnight. Water (100 mL) was added to the reaction mixture and extracted with petroleum ether (3×100 mL). The combined organic layer was washed with brine (50 mL), dried over Na₂SO₄ and concentrated under reduced pressure at 25° C. The crude was purified by Combiflash Isco (Silicycle, 120 g, silica, 100% petroleum ether) to yield dibromide 15a (3.9 g). GC/MS (Condition 16): Rₜ=7.07 min. ¹H NMR (CDCl₃, δ=7.26 ppm, 400 MHz): δ 7.17 (s, 2H), 3.09 (s, 4H). GC/MS: Anal. Calcd. for [M]⁺ C₈H₆Br₂: 261.94; found 261.9.

### Example 15

### Step b

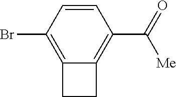

A solution dibromide 15a (5 g, 19.09 mmol) in 1,4-dioxane (50 mL) was purged with N₂ for 10 minutes. Then tributyl(1-ethoxyvinyl)tin (5.85 mL, 17.18 mmol) was added followed by Pd(Ph₃P)₂Cl₂ (0.670 g, 0.954 mmol). The reaction mixture was purged with N₂ for further 10 minutes and heated at 100° C. for 1 h under microwave conditions. The reaction mixture was filtered through a plug of diatomaceous earth (Celite®) and the filtrate was diluted with DCM (30 mL) and HCl (1.5 N, 50 mL) at room temperature. After being stirred for 2 h, the reaction mixture was extracted with DCM (100 mL), and the organic layer was washed with brine (50 mL), dried over Na₂SO₄ and concentrated in vacuo at 25° C. The crude was purified by Combiflash Isco (Redisep, 40 g, silica, 3-5% EtOAC/petroleum ether) to yield 15b (1.2 g) as a white solid. GC/MS (Condition 16): Rₜ=7.75 min. ¹H NMR (CDCl₃, δ=7.26 ppm, 400 MHz): δ 7.62 (d, J=8.4, 1H), 7.40 (d, J=8.4, 1H), 3.41 (app t, 2H), 3.21 (app t, 2H), 2.49 (s, 3H). GC/MS: Anal. Calcd. for [M]⁺ C₁₀H₉BrO: 225.08; found 225.9.

### Example 15

### Step c

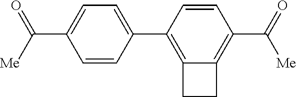

A solution of 15b (900 mg, 4.00 mmol), 4-acetylphenylboronic acid (983 mg, 6.00 mmol), K₂CO₃ (1.658 g, 12.00 mmol) in 1,4-dioxane (9 mL) and water (0.9 mL) was purged with N₂ for 10 minutes. Then Pd(Ph₃P)₄ (462 mg, 0.400 mmol) was added, the reaction mixture was purged with N₂ for further 10 minutes and heated at 100° C. for 1.5 h under microwave conditions. The reaction mixture was filtered through Celite plug and washed with EtOAC (3×10 mL). The filtrate was evaporated under reduced pressure and H₂O (50 mL) was added to the resulting residue. The crude was extracted with EtOAC (50 mL), and the organic layer was washed with brine (50 mL), dried over Na₂SO₄ and concentrated in vacuo. The crude was purified by Combiflash Isco (Redisep, 26 g, C18, 0.05% TFA in water: ACN, 51:49) to obtain 15c (430 mg) as white solid. LC/MS (Condition 14): Rₜ=1.89 min. ¹H NMR (CDCl₃, δ=7.26 ppm, 400 MHz): δ 8.06-8.04 (m, 2H), 7.87 (d, J=8.4, 1H), 7.78-7.76 (m, 2H), 7.61 (d, J=8.4, 1H), 3.53-3.50 (m, 4H), 2.64 (s, 3H), 2.54 (s, 3H). LC/MS: Anal. Calcd. for [M+H]⁺ C₁₈H₁₇O₂: 265.12; found 264.9.

### Example 15

### Step d

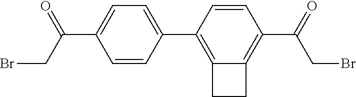

To a stirred solution of 15c (430 mg, 1.627 mmol) in 1,4-dioxane (2.5 mL), Br₂ (0.168 mL, 3.25 mmol) in 1,4-dioxane (1 mL) was added at 10° C. and stirred at room temperature for 2 h. Water (20 mL) was added to the reaction mixture and the resulting solid was collected by filtration. The solid was dried under high vacuum to obtain crude dibromide 15d (630 mg) which was used as such in the next step. LC/MS (Condition 14): Rₜ=2.04 min. LC/MS: Anal. Calcd. for [M+H]⁺ C₁₈H₁₅Br₂O₂: 423.11; found 423.1.

### Example 15

### Step e

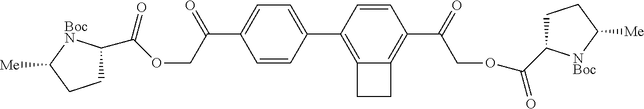

A solution of crude dibromide 15d (350 mg, 0.829 mmol) in ACN (5 mL) was cooled to 0° C. Then (2S,5S)-1-(tert-butoxycarbonyl)-5-methylpyrrolidine-2-carboxylic acid (418 mg, 1.824 mmol) was added followed by drop-wise addition of DIPEA (0.579 mL, 3.32 mmol). The reaction mixture was allowed to warm to room temperature and stirred for 2 h. The reaction mixture was diluted with EtOAc (30 mL) and washed with saturated NH₄Cl (20 mL), 10% NaHCO₃ (20 mL), water (20 mL) and brine (10 mL). The organic layer was dried over Na₂SO₄ and concentrated under reduced pressure. The crude was purified by Combiflash Isco (Silicycle, 40 g, silica, EtOAc: petroleum ether, 35:65) to afford diketoester 15e (300 mg) as a yellow solid. LC/MS (Condition 14): Rₜ=2.36 min. ¹H NMR (CDCl₃, δ=7.26 ppm, 400 MHz): δ 8.01-7.99 (m, 2H), 7.89 (d, J=8.4, 1H), 7.79-7.77 (m, 2H), 7.62 (d, J=8.4, 1H), 5.60-5.05 (m, 4H), 4.55-4.47 (m, 1H), 4.45-4.38 (m, 1H), 4.09-4.01 (m, 1H), 3.98-3.92 (m, 1H), 3.52 (br s, 4H), 2.38-2.29 (m, 4H), 2.17-2.04 (m, 2H), 1.80-1.69 (m, 2H), 1.48/1.45 (s, 18H), 1.33 (br s, 6H). LC/MS: Anal. Calcd. for [M−H]⁻ C₄₀H₄₉N₂O₁₀: 717.35; found 717.6.

### Example 15

### Step f

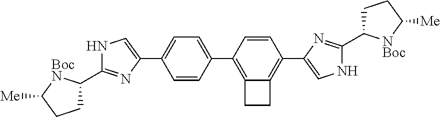

To a solution of diketoester 15e (675 mg, 0.939 mmol) in xylenes (15 mL) was added NH₄OAc (1.448 g, 18.78 mmol) and heated in a sealed tube at 130° C. for overnight. The volatile components were evaporated under reduced pressure and the resulting residue was diluted with EtOAc (50 mL) and treated with 10% NaHCO₃ (50 mL). The organic layer was separated, washed with water (50 mL), brine (25 mL), dried over Na₂SO₄ and concentrated in vacuo. The crude was purified by Combiflash Isco (Redisep, 26 g, C18, 0.01M NH₄OAc-water: ACN, 63: 37) to obtain carbamate 15f (152 mg) as a yellow solid. LC/MS (Condition 15): Rₜ=2.29 min. ¹H NMR (MeOD, δ=3.34 ppm, 400 MHz): δ 7.78 (d, J=8.4, 2H), 7.73 (d, J=8.4, 2H), 7.67 (d, J=8.4, 1H), 7.60 (d, J=8.4, 1H), 7.39 (s, 1H), 7.22 (s, 1H), 4.99-4.85 (obscured, 2H), 4.11-4.03 (m, 2H), 3.52 (app t, 2H), 3.38 (app t, 2H), 2.31-2.16 (m, 6H), 1.78-1.69 (m, 2H), 1.44-1.28 (br m, 24H). LC/MS: Anal. Calcd. for [M+H]⁺ C₄₀H₅₁N₆O₄: 679.39; found 679.4.

### Example 15

### Step g

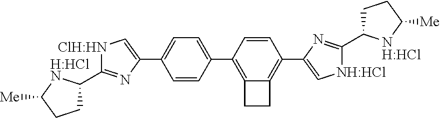

To a solution of carbamate 15f (60 mg, 0.088 mmol) in MeOH (1 mL) at 0° C. was added HCl/MeOH (4N, 1 mL) and stirred at room temperature for 2 h. The volatile component was removed in vacuo and the residue was co-evaporated with dry DCM (3×5 mL). The resulting salt was exposed to high vacuum to afford pyrrolidinine 15 g (4HCl) (52 mg) as a yellow solid which was submitted to the next step as such. LC/MS (Condition 10): Rₜ=1.23 min. ¹H NMR (DMSO-d₆, δ=2.50 ppm, 400 MHz): δ 10.34 (br s, 2H), 8.02 (s, 1H), 7.97 (d, J=8.4, 2H), 7.85-7.82 (m, 3H), 7.74 (d, J=8.4, 1H), 7.68 (s, 1H), 4.99-4.91 (br m, 2H), 3.82-3-3.77 (br m, 2H), 3.53-3.49 (m, 2H), 3.41-3.37 (m, 2H), 2.51-2.44 (m, 4H), 2.29-2.24 (m, 2H), 1.91-1.86 (m, 2H), 1.44 (d, J=6.8, 6H). LC/MS: Anal. Calcd. for [M+H]⁺ C₃₀H₃₅N₆: 479.28; found 479.3.

### Example 15

To a solution of pyrrolidine 15g (4HCl) (42.1 mg, 0.088 mmol) in DMF (2 mL) was added (S)-2-((2R,4r,6S)-2,6-dimethyltetrahydro-2H-pyran-4-yl)-2-((methoxycarbonyl)amino)acetic acid (45.3 mg, 0.185 mmol) at 0° C. Then HATU (68.6 mg, 0.180 mmol) was added followed by DIPEA (0.061 mL, 0.352 mmol). After being stirred for 1.5 h at room temperature, the volatile component was removed in vacuo and the residue was dissolved in DCM (50 mL), washed with saturated NH₄Cl (25 mL), 10% NaHCO₃ (25 mL), brine (25 mL), dried over Na₂SO₄ and concentrated in vacuo. The crude was submitted to reverse phase HPLC purification (ACN/water/NH₄OAc) to afford Example 15 (40 mg, free base) as a white solid. LC (Condition 1 and 2): >96% homogeneity index. LC/MS (Condition 10): Rₜ=1.89 min. ¹H NMR (DMSO-d₆, δ=2.50 ppm, 400 MHz): δ 12.07/12.03/11.95/11.85 (br s, 2H), 7.79-7.24 (m, 10H), 5.29/5.00 (m, 2H), 4.66/4.23 (m, 2H), 4.10/3.98 (m, 2H), 3.55 (br s, 6H), 3.45-3.11 (m, 8H), 2.35-2.10 (m, 4H), 2.06-1.86 (m, 4H), 1.82-1.58 (m, 4H), 1.51-1.18 (m, 8H), 1.11-1.01 (m, 8H), 0.96-0.68 (m, 8H). LC/MS: Anal. Calcd. for [M+H]⁺ C₅₂H₆₉N₈O₈: 933.52; found 933.5.

### Example 15.1

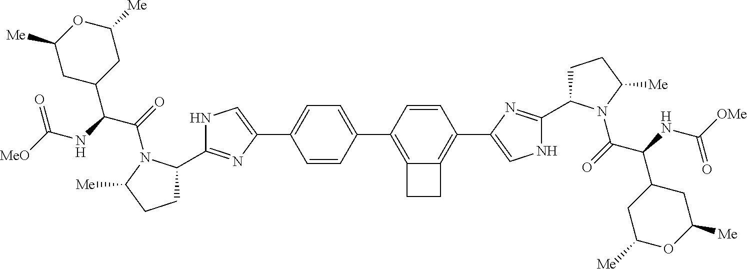

Example 15.1 was prepared from pyrrolidine 15g (4HCl) and appropriate acid by using a similar coupling condition described for Example 15. LC-MS retention time 4.076 min; m/z 933.7 (MH+). LC data was recorded on a Shimadzu LC-10AS liquid chromatograph equipped with a Phenomenex-Luna 3u C18 2.0×50 mm column using a SPD-10AV UV-Vis detector at a detector wave length of 220 nM. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 5% MeOH/95% H2O/10 mM ammonium acetate and solvent B was 5% H2O/95% MeOH/10 mM ammonium acetate. MS data was determined using a Micromass Platform for LC in electrospray mode. ¹H NMR (400 MHz, CDCl₃) δ 8.02-7.64 (m, 8H), 5.74 (d, J=5.5 Hz, 0.4H), 5.25-5.11 (m, 1.6H), 4.79-4.71 (m, 2H), 4.36-4.09 (m, 4H), 3.87-3.63 (m, 8H), 3.57 (d, J=3.3 Hz, 2H), 3.46 (br. s., 2H), 2.71-2.13 (m, 8H), 2.06-1.88 (m, 1.6H), 1.78-1.39 (m, 9.4H), 1.35-0.89 (m, 17H).

### Example 16

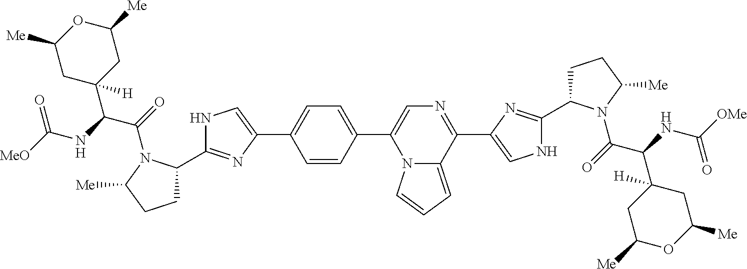

### Example 16

### Step a

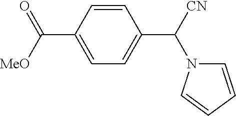

Ammonia gas purged to a stirred solution of methyl 4-formylbenzoate (5.0 g, 30.5 mmol) and Zn(OTf)₂ (2.215 g, 6.09 mmol) in THF (150 mL) at 0° C. for 5-10 minutes. After 10 minutes, TMSCN (4.90 mL, 36.6 mmol) was added at 0° C. and allowed to stir at room temperature for 1 h. The reaction mixture was quenched with water (50 mL) and extracted with EtOAc (100 mL). The organic layer was separated, washed with water (2×50 mL), brine (25 mL), dried over Na₂SO₄ and concentrated under reduced pressure. To the resulting crude cyanoamine in AcOH (50 mL) was added 2,5-dimethoxytetrahydrofuran (4.03 g, 30.5 mmol) and heated to reflux for 2 h. Then the reaction mixture was concentrated under reduced pressure and the resulting crude was diluted with DCM (100 mL). The organic layer was washed with 10% NaHCO₃ (50 mL), brine (50 mL), dried over Na₂SO₄ and concentrated in vacuo. The crude material was purified by Combiflash Isco (Redisep, 80 g, basic Al₂O₃, 20-30% EtOAc/petroleum ether) to obtain 16a (3.8 g) as pale yellow semi solid. LC/MS (Condition 10): Rₜ=1.83 min. ¹H NMR (CDCl₃, δ=7.26 ppm, 400 MHz): δ 8.10-8.08 (m, 2H), 7.40-7.38 (m, 2H), 6.76 (app t, 2H), 6.28 (app t, 2H), 6.18 (s, 1H), 3.94 (s, 3H). LC/MS: Anal. Calcd. for [M+H]⁺ C₁₄H₁₃N₂O₂: 241.09; found 241.2.

### Example 16

### Step b

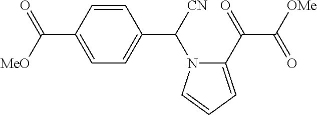

A solution of 16a (3.5 g, 14.57 mmol) and methyl oxalyl chloride (4.06 mL, 43.7 mmol) in benzene (75 mL) was heated to 95° C. for 4 h. The reaction mixture was cooled to room temperature and concentrated under reduced pressure. The crude was diluted with EtOAc (75 mL), washed with 10% NaHCO₃ (50 mL), brine (50 mL), dried over Na₂SO₄ and concentrated in vacuo to obtain the crude 16b (3.8 g) which was proceeded for next step without purification. LC/MS (Condition 10): Rₜ=1.77 min. ¹H NMR (CDCl₃, δ=7.26 ppm, 400 MHz): δ 8.09-8.07 (m, 2H), 7.78 (s, 1H), 7.52 (dd, J=4.4, 1.6, 1H), 7.45-7.43 (m, 2H), 7.31 (dd, J=2.8, 1.6, 1H), 6.42 (dd, J=4.4, 2.8, 1H), 3.94 (s, 3H), 3.93 (s, 3H). LC/MS: Anal. Calcd. for [M−H]⁻ C₁₇H₁₃N₂O₅: 325.09; found 325.2.

### Example 16

### Step c

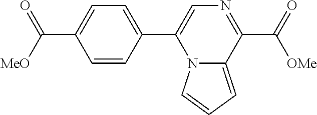

To a solution of crude 16b (3.8 g, 11.65 mmol) in MeOH (75 mL) was added Pd/C (0.496 g, 0.466 mmol) and AcOH (0.667 mL, 11.65 mmol). The reaction mixture was stirred at room temperature for 2 h under hydrogen atmosphere. The reaction mixture was filtered through diatomaceous earth (Celite®) and washed with MeOH (2×50 mL). The filtrate was concentrated under reduced pressure to obtain a mixture of crude product 16c (36%) and the unaromatized derivative (45%). The above mixture (3.6 g, 11.45 mmol) was dissolved in THF (75 mL) and heated to 70° C. Then a solution of DDQ (5.20 g, 22.91 mmol) in THF (10 mL) was added to the reaction mixture at 70° C. and allowed to stir for 2 h. Then the reaction mixture was diluted with EtOAc (100 mL), washed with water (50 mL), 10% NaHCO₃ (3×100 mL), brine (50 mL), dried over Na₂SO₄ and concentrated in vacuo. The crude was dissolved in MeOH (5 mL) and precipitated with diethyl ether:petroleum ether, 20:75 mL). The resulting precipitate was washed with diethyl ether: petroleum ether, 10:90 mL) to obtain the desired product 16c (1.2 g) as a yellow solid. LC/MS (Condition 10): Rₜ=1.77 min. ¹H NMR (CDCl₃, δ=7.26 ppm, 400 MHz): δ 8.26-8.24 (m, 2H), 7.78-7.76 (m, 2H), 7.66 (s, 1H), 7.63-7.61 (m, 2H), 7.06 (dd, J=4.4, 2.8, 1H), 4.09 (s, 3H), 3.99 (s, 3H). LC/MS: Anal. Calcd. For [M+H]⁺ C₁₇H₁₅N₂O₄: 311.10; found 311.0.

### Example 16

### Step d

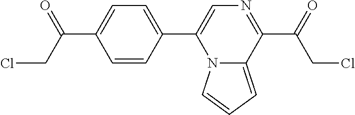

To a stirred solution of 16c (0.7 g, 2.256 mmol) in THF (70 mL) was added chloroiodomethane (1.310 mL, 18.05 mmol) at −78° C. After 5 minutes, LDA (12.53 mL, 22.56 mmol) was added drop-wise over 30 minutes. The reaction was stirred for 10 minutes and then a solution of AcOH (3.5 mL) in THF (10 mL) was added at below −65° C. The reaction was stirred for an additional 10 minutes and partitioned between EtOAc (50 mL) and brine (50 mL). The organic layer was separated and washed with 10% NaHCO₃ (50 mL), brine (50 mL), dried over Na₂SO₄ and concentrated in vacuo to obtain crude 16d (0.78 g) as a dark yellow color liquid. This crude was continued for next step without purification. LC/MS (Condition 11): Rₜ=2.09 min, Anal. Calcd. for [M−H]⁻ C₁₂H₁₁Cl₂N₂O₂: 345.03; found 345.0.

### Example 16

### Step e

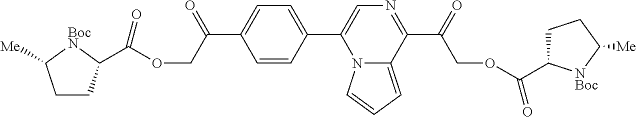

To a solution of crude 16d (350 mg, 1.008 mmol) and (2S,5S)-1-(tert-butoxycarbonyl)-5-methylpyrrolidine-2-carboxylic acid (462 mg, 2.016 mmol) in ACN (50 mL) was added KI (36.8 mg, 0.222 mmol) followed by DIPEA (0.704 mL, 4.03 mmol) at 0° C. Then the reaction mixture was stirred at RT for 2 h. The reaction mixture was diluted with EtOAc (50 mL) and washed with saturated NH₄Cl (50 mL), 10% NaHCO₃ (50 mL), brine (50 mL), dried over Na₂SO₄ and concentrated under reduced pressure. The crude was dissolved in MeOH (5 mL) and precipitated with diethyl ether:petroleum ether, 50:45 mL). The resulting precipitate was redissolved in MeOH (5 mL) and precipitated with diethyl ether:petroleum ether, 50:45 mL). This process was repeated for one more time. The precipitate was dried under high vacuum to obtain 16e (700 mg) as a yellow solid. LC/MS (Condition 10): Rₜ=2.55 Min. ¹H NMR (CDCl₃, δ=7.26 ppm, 400 MHz): δ 8.14-8.12 (m, 2H), 7.85-7.82 (m, 2H), 7.70-7.65 (m, 1H), 7.59 (s, 1H), 7.59-7.56 (m, 1H), 7.08-7.04 (m, 1H), 5.89-5.26 (m, 4H), 4.58-4.40 (m, 2H), 4.10-3.72 (m, 2H), 2.41-2.24 (m, 4H), 2.15-2.03 (m, 2H), 1.78-1.60 (m, 2H), 1.50 (br s, 18H), 1.40-1.31 (m, 6H). LC/MS: Anal. Calcd. For [M+H]⁺ C₃₉H₄₉N₄O₁₀: 733.34; found 733.4.

### Example 16

### Step f

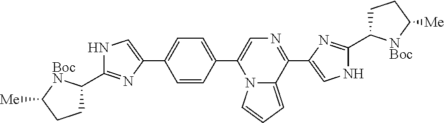

To a solution of 16e (700 mg, 0.955 mmol) in xylenes (10 mL) was added NH₄OAc (1.473 g, 19.10 mmol). The reaction mixture was purged with nitrogen for 10 minutes and then heated to 130° C. in a sealed tube for 18 h. The volatile components were evaporated under reduced pressure, the resulting residue was diluted with DCM (50 mL) and treated with saturated NaHCO₃ (50 mL). Then the organic layer was separated, washed with brine (25 mL), dried over Na₂SO₄ and concentrated in vacuo. The residue was purified by Combiflash Isco (Redisep, 26 g, C18, 50-60% ACN: 10 mM NH₄HCO₃) to obtain 16f (80 mg) as a yellow liquid. LC/MS (Condition 10): Rₜ=2.28 min. ¹H NMR (MeOD, δ=3.34 ppm, 400 MHz): δ 7.98-7.72 (m, 5H), 7.50-6.98 (m, 5H), 5.01-4.85 (obscured, 2H), 4.10-4.0 (m, 2H), 2.40-2.11 (m, 6H), 1.84-1.60 (m, 2H), 1.51-1.30 (m, 24H). LC/MS: Anal. Calcd. For [M+H]⁺ C₃₉H₄₉N₈O₄: 693.38; found: 693.4.

### Example 16

### Step g

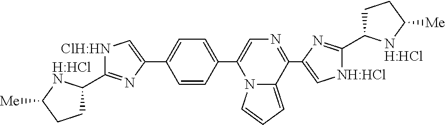

To a solution of 16f (55 mg, 0.079 mmol) in MeOH (1 mL) at 0° C. was added HCl/MeOH (4N, 1.5 mL) and stirred at room temperature for 2 h. The volatile component was removed in vacuo and the residue was co-evaporated with dry DCM (3×5 mL). The resulting salt was exposed to high vacuum to afford pyrrolidinine 16g (4HCl) (55 mg) as a yellow solid which was submitted to the next step as such. LC/MS (Condition 10): Rₜ=1.15 min. ¹H NMR (MeOD, δ=3.34 ppm, 400 MHz): δ 8.70 (s, 1H), 8.39 (dd, J=4.8, 0.8, 1H), 8.22 (dd, J=2.4, 0.8, 1H), 8.18 (d, J=8.4, 2H), 8.12 (s, 1H), 7.97 (d, J=8.4, 2H), 7.62 (s, 1H), 7.45 (dd, J=4.8, 2.8, 1H), 5.19 (app t, 1H), 5.08 (app t, 1H), 4.03-3.95 (m, 2H), 2.76-2.58 (m, 4H), 2.53-2.43 (m, 2H), 2.12-2.01 (m, 2H), 1.60 (d, J=6.4, 3H), 1.59 (d, J=6.8, 3H). LC/MS: Anal. Calcd. For [M+H]⁺ C₂₉H₃₃N₈: 493.27; found: 493.3.

### Example 16

To a solution of 16g (4HCl) (55 mg, 0.086 mmol) and (S)-2-((2R,4r,6S)-2,6-dimethyltetrahydro-2H-pyran-4-yl)-2-(methoxycarbonylamino)acetic acid (44.4 mg, 0.181 mmol) in DMF (2.5 mL) was added HATU (67.1 mg, 0.177 mmol) followed by DIPEA (0.060 mL, 0.345 mmol) at 0° C. After being stirred for 2 h at room temperature, the volatile component was removed in vacuo and the residue was dissolved in DCM (50 mL), washed with saturated NH₄Cl (25 mL), 10% NaHCO₃ (25 mL), brine (25 mL), dried over Na₂SO₄ and concentrated in vacuo. The crude was submitted to reverse phase HPLC purification (ACN/water/TFA) to afford TFA salt of Example 16 (60 mg, 0.062 mmol, 72.1% yield) as a yellow solid. LC (Condition 1 and 2): >98% homogeneity index. LC/MS (Condition 10): Rₜ=1.79 min. ¹H NMR (MeOD, δ=3.34 ppm, 400 MHz): δ 8.62/8.45 (s, 1H), 8.31-8.27 (m, 1H), 8.16-8.05 (m, 3H), 7.94-7.88 (m, 3H), 7.55-7.53 (m, 1H), 7.40-7.36 (m, 1H), 5.62/5.50/5.19 (m, 2H), 4.86-4.75 (obscured, 2H), 4.29-4.16 (m, 2H), 3.76/3.75/3.59 (s, 6H), 3.56-3.22 (m, 4H), 2.82-2.19 (m, 6H), 2.17-1.76 (m, 4H), 1.61-1.37 (m, 6H), 1.32-1.09 (m, 14H), 1.03-0.82 (m, 6H). LC/MS: Anal. Calcd. For [M+H]⁺ C₅₁H₆₇N₁₀O₈: 947.51; found: 947.4.

### Example 17

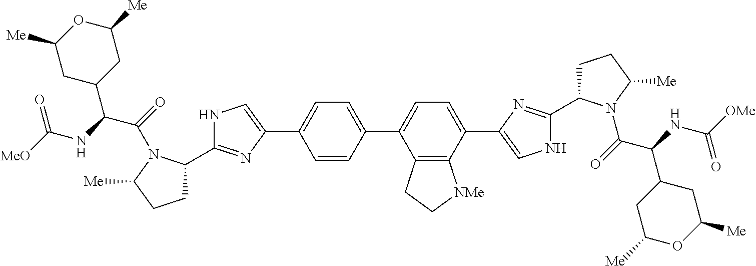

### Example 17

### Step a

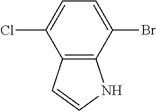

To a stirred solution of 1-bromo-4-chloro-2-nitrobenzene (10 g, 42.3 mmol) in THF (125 mL) was added vinylmagnesium bromide (1M in THF, 127 mL, 127 mmol) at −78° C. and the reaction mixture was stirred at the same temperature for 30 minutes. Then the reaction mixture was quenched with saturated NH₄Cl (60 mL). The volatile components were evaporated under reduced pressure and H₂O (100 mL) was added to the resulting residue. The crude was extracted with DCM (2×50 mL), and the organic layer was washed with brine (50 mL), dried over Na₂SO₄ and concentrated in vacuo. The residue was purified by flash chromatography (Silica gel 230-400, 1.3% EtOAc/petroleum ether) to yield 17a (4.5 g) as brown oil. LC/MS (Condition 15): Rₜ=2.01 min. ¹H NMR (DMSO-d₆, δ=2.50 ppm, 400 MHz): δ 11.72 (br s, 1H), 7.52 (t, J=2.8, 1H), 7.32 (d, J=8.0, 1H), 7.05 (d, J=8.0, 1H), 6.61-6.59 (m, 1H). LC/MS: Anal. Calcd. for [M−H]⁻ C₈H₄BrClN: 227.93; found 228.0.

### Example 17

### Step b

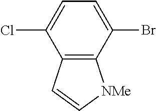

To a solution of 17a (4.5 g, 19.52 mmol) in DMF (65 mL) was added NaH (60% in mineral oil) (0.937 g, 23.43 mmol) at 0° C. and stirred at that temperature for 30 minutes. MeI (1.221 mL, 19.52 mmol) was added drop-wise to the reaction mixture and stirred at room temperature for 12 h. Water (30 mL) was added to the reaction mixture and extracted with EtOAc (2×50 mL). The organic layer was washed with brine (25 mL), dried over Na₂SO₄ and concentrated under reduced pressure. The crude was purified by Combiflash Isco (Silicycle, 40 g, silica, 100% petroleum ether) to yield 17b (4.1 g) as yellow oil. GC/MS (Condition 16): Rₜ=8.40 min. ¹H NMR (DMSO-d₆, δ=2.50 ppm, 400 MHz): δ 7.51 (d, J=3.2, 1H), 7.32 (d, J=8.4, 1H), 7.02 (d, J=8.4, 1H), 6.52 (d, J=3.2, 1H), 4.14 (s, 3H). GC/MS: Anal. Calcd. for [M]⁺ C₉H₇BrClN: 244.52; found 245.0.

### Example 17

### Step c

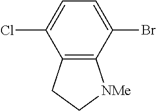

NaCNBH₃ (8.43 g, 134 mmol) was added to a solution of 17b (4.1 g, 16.77 mmol) in AcOH (30 mL) at 10° C. and the reaction mixture was stirred at room temperature for 12 h. The reaction mixture was cooled to 0° C., water (50 mL) was added to the reaction mixture and slowly basified by 10% NaOH. The reaction mixture was extracted with EtOAc (4×25 mL), the organic layer was washed with brine (25 mL), dried over Na₂SO₄ and concentrated under reduced pressure. The crude was purified by Combiflash Isco (Silicycle, 40 g, silica, 100% petroleum ether) to yield 17c (3.5 g) as colorless oil. LC/MS (Condition 9): Rₜ=2.17 min. ¹H NMR (DMSO-d₆, δ=2.50 ppm, 400 MHz): δ 7.17 (d, J=8.4, 1H), 6.58 (d, J=8.4, 1H), 3.44 (app t, 2H), 3.06 (s, 3H), 2.94 (t, J=8.8, 2H). LC/MS: Anal. Calcd. for [M+H]⁺ C₉H₁₀BrClN: 245.96; found 246.0.

### Example 17

### Step d

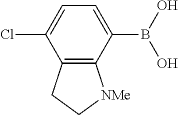

Chlorobromide 17c (1 g, 4.06 mmol) and triisopropyl borate (1.130 mL, 4.87 mmol) were dissolved in toluene (10 mL) and THF (2.5 mL) and cooled to −70° C. n-BuLi (2.0 M in hexane, 2.434 mL, 4.87 mmol) was added drop-wise to the reaction mixture and stirred at the same temperature for 1 h. The reaction mixture was then brought to the room temperature and 1.5 N HCl (5 mL) was added. The aqueous and organic layers were separated. The aqueous layer was neutralized to pH 7 using 10% NaOH solution and then extracted with EtOAc (3×15 mL). The EtOAc layer was washed with brine (15 mL), dried over Na₂SO₄ and concentrated under reduced pressure to yield 17d (500 mg) as a brown solid. LC/MS (Condition 9): Rₜ=0.97 min. ¹H NMR (DMSO-d₆, δ=2.50 ppm, 400 MHz): δ 8.10 (s, 2H), 7.00 (d, J=8.0, 1H), 6.57 (d, J=8.0, 1H), 3.36-3.34 (obscured, 2H), 2.89 (app t, 2H), 2.78 (s, 3H). LC/MS: Anal. Calcd. for [M+H]⁺ C₉H₁₂BClNO₂: 212.6; found 212.0.

### Example 17

### Step e

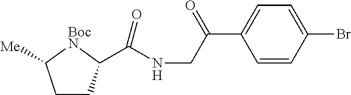

To a stirred solution of 2-amino-1-(4-bromophenyl)ethanone hydrochloride (5 g, 19.96 mmol) in DMF (50 mL) was added (2S,5S)-1-(tert-butoxycarbonyl)-5-methylpyrrolidine-2-carboxylic acid (4.80 g, 20.96 mmol) at 0° C. Then HATU (7.74 g, 20.36 mmol) was added to the reaction mixture followed by DIPEA (10.46 mL, 59.9 mmol). The reaction mixture was allowed to warm to room temperature and stirred for 1.5 h. Water (100 mL) was added to the reaction mixture and extracted with EtOAc (2×100 mL). The organic layer was washed with brine (50 mL), dried over Na₂SO₄ and concentrated under reduced pressure. The crude product was purified by flash chromatography (Silica gel 60-120, 1.8% MeOH/DCM) to yield 17e (7.9 g) as an off white solid. LC/MS (Condition 15): Rₜ=1.99 min. ¹H NMR (CDCl₃, δ=7.26 ppm, 400 MHz): δ 7.84 (d, J=8.8, 2H), 7.65 (d, J=8.8, 2H), 4.84-4.62 (m, 2H), 4.39 (br s, 1H), 3.94 (br s, 1H), 2.23 (br s, 1H), 2.10-2.02 (m, 2H), 1.64-1.54 (m, 1H), 1.48/1.47 (s, 9H), 1.39 (d, J=6.0, 3H). LC/MS: Anal. Calcd. for [M−H]⁻ C₁₉H₂₄BrN₂O₄: 424.32; found 425.0.

### Example 17

### Step f

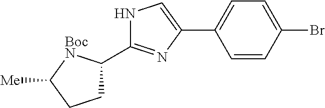

To a solution of 17e (4 g, 9.40 mmol) in xylenes (30 mL) was added NH₄OAc (3.62 g, 47.0 mmol) and heated in a sealed tube at 130° C. for 12 h. The volatile components were evaporated under reduced pressure and the reaction mixture was treated with 10% NaHCO₃ (25 mL). Then the reaction mixture was extracted with EtOAc (2×50 mL), and the organic layer was washed with water (50 mL), brine (25 mL), dried over Na₂SO₄ and concentrated in vacuo. The crude was purified by Combiflash Isco (Silicycle, 120 g, silica gel, 1-2% MeOH/CHCl₃) to yield 17f (2.8 g) as a yellow solid. LC/MS (Condition 15): Rₜ=2.07 min. ¹H NMR (CDCl₃, δ=7.26 ppm, 400 MHz): δ 10.87/10.42 (br s, 1H), 7.65 (d, J=8.0, 2H), 7.46 (d, J=8.0, 2H), 7.22 (s, 1H), 4.96-4.93 (m, 1H), 3.97-3.93 (m, 1H), 3.08-2.88 (m, 1H), 2.22-2.10 (m, 2H), 1.90-1.78 (m, 1H), 1.50/1.48 (s, 9H), 1.16 (br s, 3H). LC/MS: Anal. Calcd. for [M+H]⁺ C₁₉H₂₅BrN₃O₂: 406.11; found 406.2.

### Example 17

### Step g

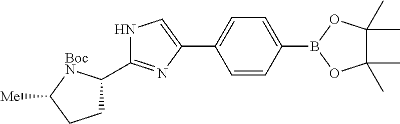

A solution of 17f (1 g, 2.461 mmol), bis(pinacolato)diboron (1.312 g, 5.17 mmol) and KOAc (0.604 g, 6.15 mmol) in 1,4-dioxane (20 mL) was purged with N₂ for 10 minutes. Then Pd(Ph₃P)₄ (0.142 g, 0.123 mmol) was added, the reaction mixture was purged with N₂ for further 10 minutes and heated at 80° C. for 12 h. The reaction mixture was filtered through Celite plug and washed with EtOAc (2×10 mL). The filtrate was evaporated under reduced pressure and H₂O (50 mL) was added to the resulting residue. The crude was extracted with EtOAc (50 mL), and the organic layer was washed with brine (50 mL), dried over Na₂SO₄ and concentrated in vacuo. The crude product was purified by flash chromatography (Silica gel 230-400, 2.1% MeOH/DCM) to yield 17g (583 mg, 41.7% yield) as a yellow solid. LC/MS (Condition 15): Rₜ=2.15 min. ¹H NMR (CDCl₃, δ=7.26 ppm, 400 MHz): δ 10.90/10.40 (br s, 1H), 7.83-7.40 (m, 4H), 7.27 (s, 1H), 4.97 (br s, 1H), 3.97 (br s, 1H), 3.01-2.88 (m, 1H), 2.19-2.03 (m, 2H), 1.88-1.75 (m, 1H), 1.51/1.35 (s, 9H), 1.26/1.24 (s, 12H), 1.15 (br s, 3H). LC/MS: Anal. Calcd. for [M+H]⁺ C₂₅H₃₇BN₃O₄: 454.28; found 454.4.

### Example 17

### Step h

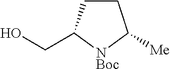

To a solution of (2S,5S)-1-(tert-butoxycarbonyl)-5-methylpyrrolidine-2-carboxylic acid (3.5 g, 15.27 mmol) in THF (10 mL) was added BH₃.DMS (1.595 mL, 16.79 mmol) at 0° C. and then refluxed at 80° C. for 12 h. The reaction mixture was cooled to 0° C. and added slowly MeOH (10 mL). After stirring for 10 minutes the volatile components were removed under reduced pressure. The resulting residue was dissolved in EtOAc (50 mL), washed with 10% NaHCO₃ (25 mL), water (25 mL), brine (10 mL), dried over Na₂SO₄ and concentrated in vacuum. The crude was purified by flash chromatography (Silica gel 230-400, 30-40% EtOAc/petroleum ether) to obtain 17h (3.1 g) as a colorless liquid. LC/MS (Condition 15): Rₜ=1.71 min. ¹H NMR (CDCl₃, δ=7.26 ppm, 400 MHz): δ 4.95 (br s, 1H), 3.98-3.88 (m, 2H), 3.71-3.63 (m, 1H), 3.57-3.50 (m, 1H), 2.03-1.89 (m, 2H), 1.72-1.50 (m, 2H), 1.47 (s, 9H), 1.16 (d, J=6.0, 3H). LC/MS: Anal. Calcd. for [M+H-Boc]⁺ C₆H₁₄NO: 116.10; found 116.2.

### Example 17

### Step i

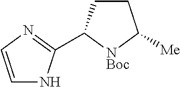

Dess-Martin Periodinane (7.39 g, 17.42 mmol) was added to a solution of 17h (2.5 g, 11.61 mmol) in DCM (50 mL) at 0° C. and stirred at RT for 2 h. The reaction mixture was dissolved in EtOAc (150 mL), washed with 10% NaHCO₃ (75 mL), 10% Na₂S₂O₃ (75 mL), brine (25 mL), dried over Na₂SO₄ and concentrated in vacuum to obtain crude (2S,5S)-tert-butyl 2-formyl-5-methylpyrrolidine-1-carboxylate (2.62 g) as a brown liquid which was used as such in the next step. ¹H NMR (CDCl₃, δ=7.26 ppm, 400 MHz): δ 9.48/9.39 (s, 1H), 4.20-3.90 (m, 2H), 2.01-1.83 (m, 3H), 1.65-1.52 (m, 1H), 1.47/1.46 (s, 9H), 1.24 (d, J=5.2, 3H). To a solution of crude (2S,5S)-tert-butyl 2-formyl-5-methylpyrrolidine-1-carboxylate (2.62 g, 12.28 mmol) in MeOH (40 mL) was added NH₄OH (9.13 mL, 234 mmol) at RT followed by drop-wise addition of glyoxal hydrate (0.57 mL, 12.28 mmol) and stirred at RT for 12 h. The volatile components were removed under reduced pressure and the resulting crude was purified by Combiflash Isco (Redisep, silica gel, 40 g, 2-3% MeOH/CHCl₃) to obtain 17i (770 mg) as a pale yellow color liquid. LC/MS (Condition 15): Rₜ=1.60 min. ¹H NMR (CDCl₃, δ=7.26 ppm, 400 MHz): δ 10.40 (br s, 1H), 6.96 (s, 2H), 4.94-4.91 (m, 1H), 3.95-3.91 (m, 1H), 2.90 (br s, 1H), 2.20-2.06 (m, 2H), 1.81 (br s, 1H), 1.48 (s, 9H), 1.12 (br s, 3H). LC/MS: Anal. Calcd. for [M+H]⁺ C₁₃H₂₂N₃O₂: 252.16; found 252.2.

### Example 17

### Step j

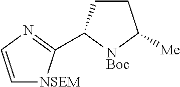

To a stirred solution of 17i (770 mg, 3.06 mmol) in DMF (10 mL) was added NaH (60% in mineral oil, 129 mg, 3.22 mmol) at 0° C. and stirred for 20 minutes. Then SEM-Cl (0.598 mL, 3.37 mmol) was added drop wise and stirred from 0° C. to RT over a period of 2 h. The reaction mixture was quenched with water (5 mL). The reaction mixture was extracted with EtOAc (50 mL), washed with water (25 mL), brine (10 mL), dried over Na₂SO₄ and concentrated in vacuum. The crude was purified by flash chromatography (Silica gel 230-400, 25-30% EtOAc/petroleum ether) to obtain 171 (520 mg) as a pale yellow liquid. LC/MS (Condition 15): Rₜ=2.17 min. ¹H NMR (CDCl₃, δ=7.26 ppm, 400 MHz): δ 7.01 (br s, 1H), 6.87 (d, J=1.2, 1H), 5.76 (br s, 1H), 5.16 (d, J=11.2, 1H), 4.90 (br s, 1H), 3.95 (br s, 1H), 3.47 (app t, 2H), 2.26-2.04 (m, 4H), 1.48-1.20 (br s, 12H), 0.96-0.80 (m, 2H), −0.02 (s, 9H). LC/MS: Anal. Calcd. for [M+H]⁺ C₁₉H₃₆N₃O₃Si: 382.24; found 382.4.

### Example 17

### Step k

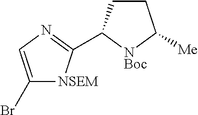

A solution of NBS (0.466 g, 2.62 mmol) in ACN (10 mL) was added drop-wise to a stirred solution of 17j (1 g, 2.62 mmol) in DCM (20 mL) at room temperature. After stirring for 1 h, water (10 mL) was added to the reaction mixture and extracted with DCM (2×30 mL). The organic layer was washed with 10% NaHCO₃ (30 mL) and brine (30 mL), dried over Na₂SO₄ and concentrated under reduced pressure. The crude was purified by flash chromatography (Silica gel 60-120, 12% EtOAc/petroleum ether) to yield 17k (600 mg) as yellow oil. LC/MS (Condition 15): Rₜ=2.41 min. ¹H NMR (CDCl₃, δ=7.26 ppm, 400 MHz): δ 6.99 (s, 1H), 5.86/5.40 (br s, 1H), 5.28 (d, J=11.2, 1H), 5.03-4.82 (m, 1H), 4.04-3.90 (m, 1H), 3.54 (app t, 2H), 2.23-2.04 (m, 4H), 1.47-1.20 (br s, 12H), 0.99-0.81 (m, 2H), −0.07 (s, 9H). LC/MS: Anal. Calcd. for [M+H]⁺ C₁₉H₃₅BrN₃O₃Si: 460.16; found 460.2.

### Example 17

### Step 1

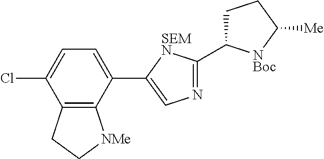

A solution of 17k (294 mg, 0.638 mmol), 17d (135 mg, 0.638 mmol), K₂CO₃ (265 mg, 1.915 mmol) in 1,4-dioxane (3 mL) and water (0.6 mL) was purged with N₂ for 10 minutes. Then Pd(Ph₃P)₄ (36.9 mg, 0.032 mmol) was added, the reaction mixture was purged with N₂ for further 10 minutes and heated under microwave at 80° C. for 2 h. The reaction mixture was filtered through a plug of diatomaceous earth (Celite®) and washed with EtOAc (2×10 mL). The filtrate was evaporated under reduced pressure and H₂O (30 mL) was added to the resulting residue. The crude was extracted with EtOAC (50 mL), and the organic layer was washed with brine (20 mL), dried over Na₂SO₄ and concentrated in vacuo. The crude was purified by Combiflash Isco (Silicycle, 40 g, silica, 12-15% EtOAc/petroleum ether) to yield 171 (124 mg) as yellow oil. LC/MS (Condition 15): Rₜ=2.88 min. ¹H NMR (CDCl₃, δ=7.26 ppm, 400 MHz): δ 6.94 (s, 1H), 6.85 (d, J=8.0, 1H), 6.64 (d, J=8.0, 1H), 5.89 (br s, 1H), 4.99 (app t, 1H), 4.88 (d, J=11.2, 1H), 4.05-3.96 (m, 1H), 3.57-3.44 (m, 1H), 3.33-3.19 (m, 2H), 3.15-2.92 (m, 3H), 2.28 (br s, 3H), 2.21-1.96 (m, 4H), 1.41/1.37 (s, 9H), 1.29-1.26 (m, 3H), 0.91-0.68 (m, 2H), −0.08 (s, 9H). LC/MS: Anal. Calcd. for [M+H]⁺ C₂₈H₄₄ClN₄O₃Si: 547.28; found 547.4.

### Example 17

### Step m

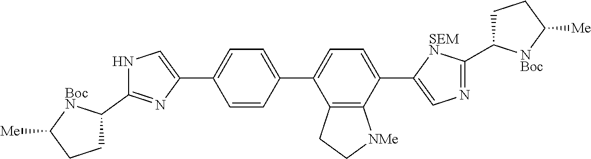

Examples 171(50 mg, 0.091 mmol) and 17g (83 mg, 0.183 mmol) were dissolved in DMF (2 mL). N₂ was purged through the reaction mixture for 10 minutes. 2-Dicyclohexylphosphino-2′,6′-dimethoxy-1,1′-biphenyl (7.50 mg, 0.018 mmol), Pd(OAc)₂ (2.051 mg, 9.14 mmol), K₂CO₃ (37.9 mg, 0.274 mmol) were added and N₂ was purged through the reaction mixture for further 10 minutes. The vessel was sealed and heated to 125° C. for 12 h. The reaction mixture was filtered through a Celite plug and washed with EtOAc (2×5 mL). The filtrate was diluted with EtOAc (10 mL), washed with water (10 mL) and brine (10 mL). The organic layer was dried over Na₂SO₄ and concentrated under reduced pressure. The crude was purified by reverse phase HPLC (ACN/water/NH₄OAc) to yield 17m (48 mg) as an off white solid. LC/MS (Condition 14): Rₜ=2.58 min. LC/MS: Anal. Calcd. for [M+H]⁺ C₄₇H₆₈N₇O₅Si: 838.50; found 838.4.

### Example 17

### Step n

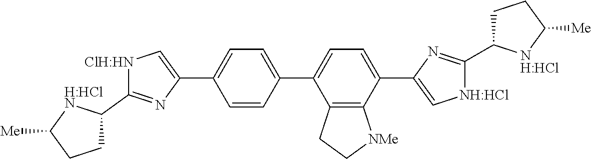

To a solution of 17m (37 mg, 0.044 mmol) in MeOH (2 mL) at 0° C. was added HCl/MeOH (4N, 8 mL) and stirred at room temperature for 12 h. The volatile component was removed in vacuo, and the residue was co-evaporated with dry DCM (3×5 mL). The resulting salt was exposed to high vacuum to afford 17n (4HCl) (23 mg) as a yellow solid which was submitted to the next step as such. LC/MS (Condition 9): Rₜ=1.07 min. ¹H NMR (MeOD, δ=3.34 ppm, 400 MHz): δ 8.08 (br s, 1H), 8.04-7.98 (m, 2H), 7.94 (br s, 1H), 7.91 (d, J=8.0, 1H), 7.78-7.72 (m, 2H), 7.68 (d, J=8.0, 1H), 5.21 (br s, 2H), 4.11 (br s, 2H), 4.02-3.90 (m, 2H), 3.60-3.51 (m, 2H), 3.37 (obscured, 3H), 2.78-2.58 (m, 3H), 2.52-2.40 (m, 3H), 2.12-2.00 (m, 2H), 1.63 (d, J=5.6, 3H), 1.59 (d, J=6.0, 3H). LC/MS: Anal. Calcd. for [M+H]⁺ C₃₁H₃₈N₇: 508.31; found 508.2.

### Example 17

To a solution of 17n (60 mg, 0.118 mmol) in DMF (2 mL) was added (S)-2-((2R,6R)-2,6-dimethyltetrahydro-2H-pyran-4-yl)-2-((methoxycarbonyl)amino)acetic acid (60.9 mg, 0.248 mmol), HATU (92 mg, 0.242 mmol) followed by DIPEA (0.083 mL, 0.473 mmol) at 0° C. After being stirred for 1.5 h at room temperature, the volatile component was removed in vacuo and the residue was dissolved in DCM (20 mL), washed with saturated NH₄Cl (10 mL), 10% NaHCO₃ solution (10 mL), brine (10 mL), dried over Na₂SO₄ and concentrated in vacuo. The crude was submitted to reverse phase HPLC purification (ACN/water/TFA) to afford TFA salt of Example 17 (65 mg) as a white solid. LC (Condition 1 and 7): >92% homogeneity index. LC/MS (Condition 18): Rₜ=1.63 min. LC/MS: Anal. Calcd. For [M+H]⁺ C₅₃H₇₂N₉O₈: 962.54; found: 962.4.

### Example 18

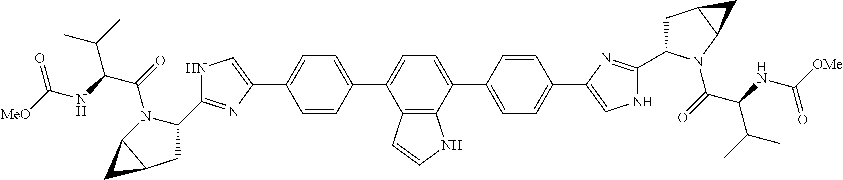

### Example 18

### Step a

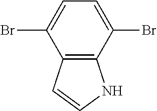

To a stirred solution of 1,4-dibromo-2-nitrobenzene (10 g, 35.8 mmol) in THF (100 mL) was added vinylmagnesium bromide (1M in THF, 107.5 mL, 107.5 mmol) at −40° C. and the reaction mixture was stirred at the same temperature for 30 minutes. Then the reaction mixture was quenched with saturated NH₄Cl (60 mL). The volatile components were evaporated under reduced pressure and H₂O (100 mL) was added to the resulting residue. The crude was extracted with DCM (2×100 mL), and the organic layer was washed with brine (50 mL), dried over Na₂SO₄ and concentrated in vacuo. The residue was purified by flash chromatography (Silica gel 230-400, 3-5% EtOAc/petroleum ether) to yield 18a (4 g). ¹H NMR (DMSO-d₆, δ=2.50 ppm, 400 MHz): δ 11.75 (s, 1H), 7.54 (t, J=2.8, 1H), 7.27 (d, J=8.4, 1H), 7.18 (d, J=8.4, 1H), 6.54-6.52 (m, 1H).

### Example 18

### Step b

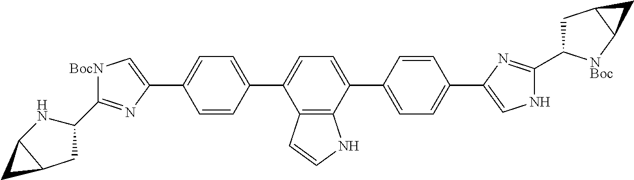

A solution of 18a (250 mg, 0.916 mmol), 1S,3R,5S)-tert-butyl 3-(4-(4-(4,4,5,5-tetramethyl-1,3,2-dioxaborolan-2-yl)phenyl)-1H-imidazol-2-yl)-2-azabicyclo[3.1.0]hexane-2-carboxylate (for preparation, see US2009-0202478 A1; 1.03 g, 2.29 mmol), and K₂CO₃ (758 mg, 5.49 mmol) in 1,4-dioxane (5 mL) and water (2 mL) was purged with N₂ for 10 minutes. Then Pd(dppf)Cl₂:DCM adduct (74.7 mg, 0.091 mmol) was added, the reaction mixture was purged with N₂ for further 10 minutes and heated at 80° C. for 12 h in a pressure tube. The reaction mixture was filtered through Celite plug and washed with EtOAc (2×5 mL). The filtrate was evaporated under reduced pressure and H₂O (10 mL) was added to the resulting residue. The crude was extracted with EtOAC (25 mL), and the organic layer was washed with brine (5 mL), dried over Na₂SO₄ and concentrated in vacuo. The crude was purified by reverse phase HPLC (ACN/water/NH₄OAc) to yield 18b (150 mg); the regioisomeric mono-coupled products were the dominant components. ¹H NMR (DMSO-d₆, δ=2.50 ppm, 400 MHz): δ 11.16 (s, 1H), 7.91 (d, J=8.4, 2H), 7.87 (d, J=8.4, 2H), 7.69 (d, J=8.4, 2H), 7.67 (d, J=8.4, 2H), 7.53 (s, 1H), 7.50 (s, 1H), 7.41 (t, J=2.8, 1H), 7.25 (d, J=7.6, 1H), 7.22 (d, J=7.6, 1H), 6.71-6.69 (m, 1H), 4.70-4.60 (m, 2H), 3.48-3.41 (m, 2H), 2.42-2.22 (m, 4H), 1.70-1.62 (m, 2H), 1.42-1.18 (m, 18H), 0.80-0.74 (m, 2H), 0.60-0.53 (m, 2H).

### Example 18

### Step c

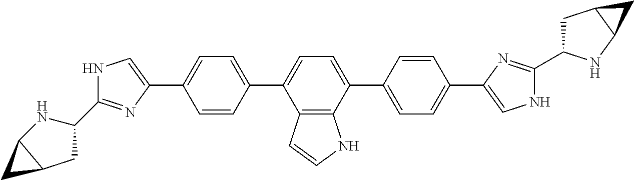

To a solution of 18b (150 mg, 0.196 mmol) in DCM (25 mL) was added TFA (1 mL) at 0° C. and stirred at room temperature for 12 h. The volatile component was removed in vacuo and the residue was co-evaporated with dry DCM (3×5 mL). The resulting salt was exposed to high vacuum to afford pyrrolidinine 18c (140 mg) which was submitted to the next step as such. LC/MS (Condition 9): Rₜ=1.41 min. ¹H NMR (DMSO-d₆, δ=2.50 ppm, 400 MHz): δ 11.19 (s, 1H), 10.08 (br s, 2H), 7.97-7.92 (m, 4H), 7.81 (s, 1H), 7.78 (s, 1H), 7.75-7.71 (m, 4H), 7.42 (t, J=2.8, 1H), 7.28-7.21 (obscured, 2H), 6.70 (app t, 1H), 4.66-4.62 (m, 2H), 3.62-3.59 (m, 2H), 3.40-3.36 (m, 2H), 1.93-1.91 (m, 2H), 1.78-1.75 (m, 2H), 1.20-1.11 (m, 2H), 0.87-0.81 (m, 2H). LC/MS: Anal. Calcd. For [M+H]⁺ C₃₆H₃₄N₇: 564.28; found: 564.2.

### Example 18

To a solution of 18c (70 mg, 0.124 mmol) and (S)-2-((methoxycarbonyl)amino)-3-methylbutanoic acid (45.7 mg, 0.261 mmol) in DMF (10 mL) was added HATU (96.9 mg, 0.254 mmol) followed by DIPEA (0.17 mL, 0.994 mmol) at 0° C. After being stirred for 4 h at room temperature, the volatile component was removed in vacuo and the residue was dissolved in DCM (50 mL), washed with saturated NH₄Cl (25 mL), 10% NaHCO₃ (25 mL), brine (25 mL), dried over Na₂SO₄ and concentrated in vacuo. The crude was submitted to reverse phase HPLC purification (ACN/water/TFA) to afford TFA salt of Example 18 (10 mg). LC (Condition 2): >91% homogeneity index. LC/MS (Condition 17): Rₜ=1.82 min. LC/MS: Anal. Calcd. For [M−H]⁻ C₅₀H₅₄N₉O₆: 877.03; found: 877.4.

### Biological Activity

An HCV Replicon assay was utilized in the present disclosure, and was prepared, conducted and validated as described in commonly owned PCT/US2006/022197 and in O'Boyle et. al. Antimicrob Agents Chemother. 2005 April; 49(4):1346-53. Assay methods incorporating luciferase reporters have also been used as described (Apath.com).

HCV-neo replicon cells and replicon cells containing resistance substitutions in the NSSA region were used to test the currently described family of compounds. The compounds were determined to have differing degrees of reduced inhibitory activity on cells containing mutations vs. the corresponding inhibitory potency against wild-type cells. Thus, the compounds of the present disclosure can be effective in inhibiting the function of the HCV NSSA protein and are understood to be as effective in combinations as previously described in application PCT/US2006/022197 and commonly owned WO/04014852. It should be understood that the compounds of the present disclosure can inhibit multiple genotypes of HCV. Table 2 shows the EC₅₀ (Effective 50% inhibitory concentration) values of representative compounds of the present disclosure against the HCV 1b genotype. Ranges are as follows: A =0.5 pM to 10 pM; B=11 pM to 90 pM; C=91 pM to 160 pM; D=161 oM to 5 nM. In one embodiment, compounds of the present disclosure are inhibitory versus 1a, 1b, 2a, 2b, 3a, 4a, and 5a genotypes.

TABLE 2

| Example | EC50 (μM) | Range |
|---|---|---|
| 1 | | A |
| 2 | | B |
| 3 | | A |
| 4 | | B |
| 5 | | C |
| 6 | | A |
| 7 | 9.42E−05 | C |
| 7.1 | | A |
| 8 | | A |
| 9 | | A |
| 10 | | A |
| 11 | | A |
| 12 | 4.09E−05 | B |
| 13 | 4.18E−03 | D |
| 14 | | D |
| 15 | | A |
| 15.1 | | A |
| 16 | | A |
| 18 | 2.74E−06 | A |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

The compounds of the present disclosure may inhibit HCV by mechanisms in addition to or other than NS5A inhibition. In one embodiment the compounds of the present disclosure inhibit HCV replicon and in another embodiment the compounds of the present disclosure inhibit NS5A. Compounds of the present disclosure may inhibit multiple genotypes of HCV.

What is claimed is:

1. A compound of Formula (I) 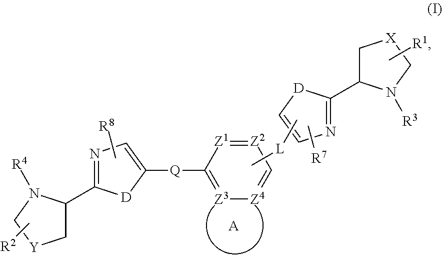
or a pharmaceutically acceptable salt thereof, wherein
each D is independently selected from O and NH;
L is a bond or phenyl;
Q is selected from phenyl, a six-membered heteroaromatic ring containing one, two, or three nitrogen atoms, and 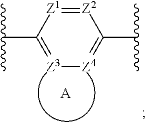
X is selected from O, S, CH₂, CH₂CH₂, (NR¹)CH₂, and OCH₂,
Y is selected from O, S, CH₂, CH₂CH₂, (NR²)CH₂, and OCH₂;
Z¹ and Z² are each independently selected from CH and N;
Z³ and Z⁴ are each independently selected from C and N;
provided that no more than two of Z¹, Z², Z³, and Z⁴ are N;
A is a four- to six-membered ring optionally containing one or two additional double bonds and optionally containing one, two, or three heteroatoms independently selected from nitrogen, oxygen, and sulfur, wherein said ring is optionally substituted with an alkyl group;
R¹ and R² are independently selected from hydrogen, alkyl, halo, and hydroxy; wherein the alkyl can optionally form a fused three- to six-membered ring or a bridged four- or five-membered ring with another carbon atom on the ring; or can optionally form a spirocyclic three- to six-membered ring with the carbon to which it is attached;
provided that when X is (NR¹)CH₂, R¹ is hydrogen or alkyl; and
provided that when Y is (NR²)CH₂, R² is hydrogen or alkyl;
R³ is selected from hydrogen and —C(O)R⁵;
R⁴ is selected from hydrogen and —C(O)R⁶;
R⁵ and R⁶ are independently selected from alkoxy, alkyl, arylalkoxy, arylalkyl, cycloalkyl, cycloalkyloxy, heterocyclyl, heterocyclylalkyl, (NR^{c}R^{d})alkenyl, and (NR^{c}R^{d})alkyl;
R⁷ and R⁸ are independently selected from hydrogen, alkyl, cyano, and halo;
R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro;
R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR^{x}R^{y})alkyl, and (NR^{x}R^{y})carbonyl; and
R^{x} and R^{y} are independently selected from hydrogen and alkyl.

2. A compound of claim 1, or a pharmaceutically acceptable salt thereof, wherein Q is phenyl.

3. A compound of claim 1, or a pharmaceutically acceptable salt thereof, wherein X and Y are each CH₂.

4. A compound of claim 1, or a pharmaceutically acceptable salt thereof, wherein R⁷ and R⁸ are each hydrogen.

5. A compound of formula (II) 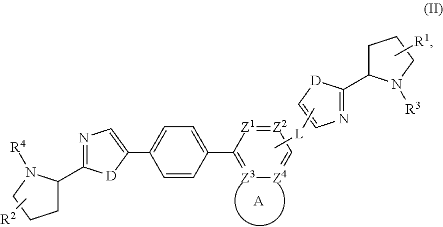
or a pharmaceutically acceptable salt thereof, wherein
each D is independently selected from O and NH;
L is a bond or phenyl;
Z¹ and Z² are each independently selected from CH and N;
Z³ and Z⁴ are each independently selected from C and N;
provided that no more than two of Z¹, Z², Z³, and Z⁴ are N;
A is a four- to six-membered ring optionally containing one or two additional double bonds and optionally containing one, two, or three heteroatoms independently selected from nitrogen, oxygen, and sulfur;
R¹ and R² are independently selected from hydrogen, alkyl, halo, and hydroxy; wherein the alkyl can optionally form a fused three- to six-membered ring or a bridged four- or five-membered ring with an another carbon atom on the ring; or can optionally form a spirocyclic three- to six-membered ring with the carbon to which it is attached;
R³ is selected from hydrogen and —C(O)R⁵;
R⁴ is selected from hydrogen and —C(O)R⁶;
R⁵ and R⁶ are independently selected from alkoxy, alkyl, arylalkoxy, arylalkyl, cycloalkyl, cycloalkyloxy, heterocyclyl, heterocyclylalkyl, (NR^{c}R^{d})alkenyl, and (NR^{c}R^{d})alkyl;
R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro;
R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR^{x}R^{y})alkyl, and (NR^{x}R^{y})carbonyl; and
R^{x} and R^{y} are independently selected from hydrogen and alkyl.

6. A compound of formula (III) 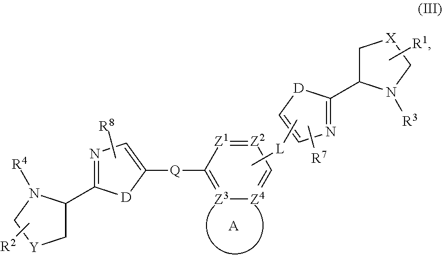
or a pharmaceutically acceptable salt thereof, wherein
each D is independently selected from O and NH;
Q is selected from phenyl, a six-membered heteroaromatic ring containing one, two, or three nitrogen atoms, and 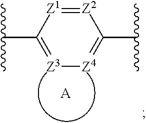
X is selected from O, S, CH₂, CH₂CH₂, (NR¹)CH₂, and OCH₂,
Y is selected from O, S, CH₂, CH₂CH₂, (NR²)CH₂, and OCH₂;
Z¹ and Z² are each independently selected from CH and N;
Z³ and Z⁴ are each independently selected from C and N;
provided that no more than two of Z¹, Z², Z³, and Z⁴ are N;
A is a four- to six-membered ring optionally containing one or two additional double bonds and optionally containing one, two, or three heteroatoms independently selected from nitrogen, oxygen, and sulfur;
R¹ and R² are independently selected from hydrogen, alkyl, halo, and hydroxy; wherein the alkyl can optionally form a fused three- to six-membered ring or a bridged four- or five-membered ring with an another carbon atom on the ring; or can optionally form a spirocyclic three- to six-membered ring with the carbon to which it is attached;
provided that when X is (NR¹)CH₂, R¹ is hydrogen or alkyl; and
provided that when Y is (NR²)CH₂, R² is hydrogen or alkyl;
R³ is selected from hydrogen and —C(O)R⁵;
R⁴ is selected from hydrogen and —C(O)R⁶;
R⁵ and R⁶ are independently selected from alkoxy, alkyl, arylalkoxy, arylalkyl, cycloalkyl, cycloalkyloxy, heterocyclyl, heterocyclylalkyl, (NR^{c}R^{d})alkenyl, and (NR^{c}R^{d})alkyl;
R⁷ and R⁸ are independently selected from hydrogen, alkyl, cyano, and halo;
R^{c} and R^{d} are independently selected from hydrogen, alkenyloxycarbonyl, alkoxyalkylcarbonyl, alkoxycarbonyl, alkyl, alkylcarbonyl, alkylsulfonyl, aryl, arylalkoxycarbonyl, arylalkyl, arylalkylcarbonyl, arylcarbonyl, aryloxycarbonyl, arylsulfonyl, cycloalkyl, cycloalkyloxycarbonyl, cycloalkylsulfonyl, formyl, haloalkoxycarbonyl, heterocyclyl, heterocyclylalkoxycarbonyl, heterocyclylalkyl, heterocyclylalkylcarbonyl, heterocyclylcarbonyl, heterocyclyloxycarbonyl, hydroxyalkylcarbonyl, (NR^{e}R^{f})alkyl, (NR^{e}R^{f})alkylcarbonyl, (NR^{e}R^{f})carbonyl, (NR^{e}R^{f})sulfonyl, —C(NCN)OR′, and —C(NCN)NR^{x}R^{y}, wherein R′ is selected from alkyl and unsubstituted phenyl, and wherein the alkyl part of the arylalkyl, the arylalkylcarbonyl, the heterocyclylalkyl, and the heterocyclylalkylcarbonyl are further optionally substituted with one —NR^{e}R^{f} group; and wherein the aryl, the aryl part of the arylalkoxycarbonyl, the arylalkyl, the arylalkylcarbonyl, the arylcarbonyl, the aryloxycarbonyl, and the arylsulfonyl, the heterocyclyl, and the heterocyclyl part of the heterocyclylalkoxycarbonyl, the heterocyclylalkyl, the heterocyclylalkylcarbonyl, the heterocyclylcarbonyl, and the heterocyclyloxycarbonyl are further optionally substituted with one, two, or three substituents independently selected from alkoxy, alkyl, cyano, halo, haloalkoxy, haloalkyl, and nitro;
R^{e} and R^{f} are independently selected from hydrogen, alkyl, unsubstituted aryl, unsubstituted arylalkyl, unsubstituted cycloalkyl, unsubstituted (cyclolalkyl)alkyl, unsubstituted heterocyclyl, unsubstituted heterocyclylalkyl, (NR^{x}R^{y})alkyl, and (NR^{x}R^{y})carbonyl; and
R^{x} and R^{y} are independently selected from hydrogen and alkyl.

7. A compound selected from 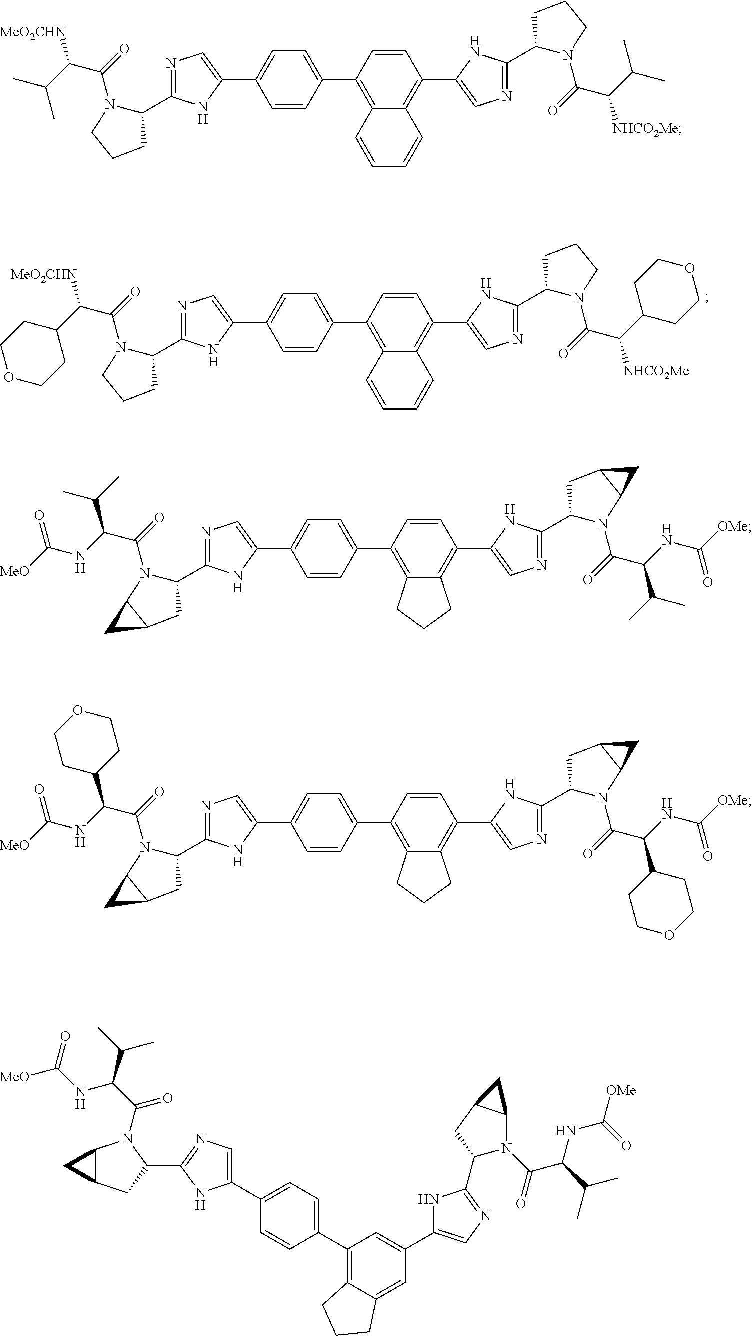 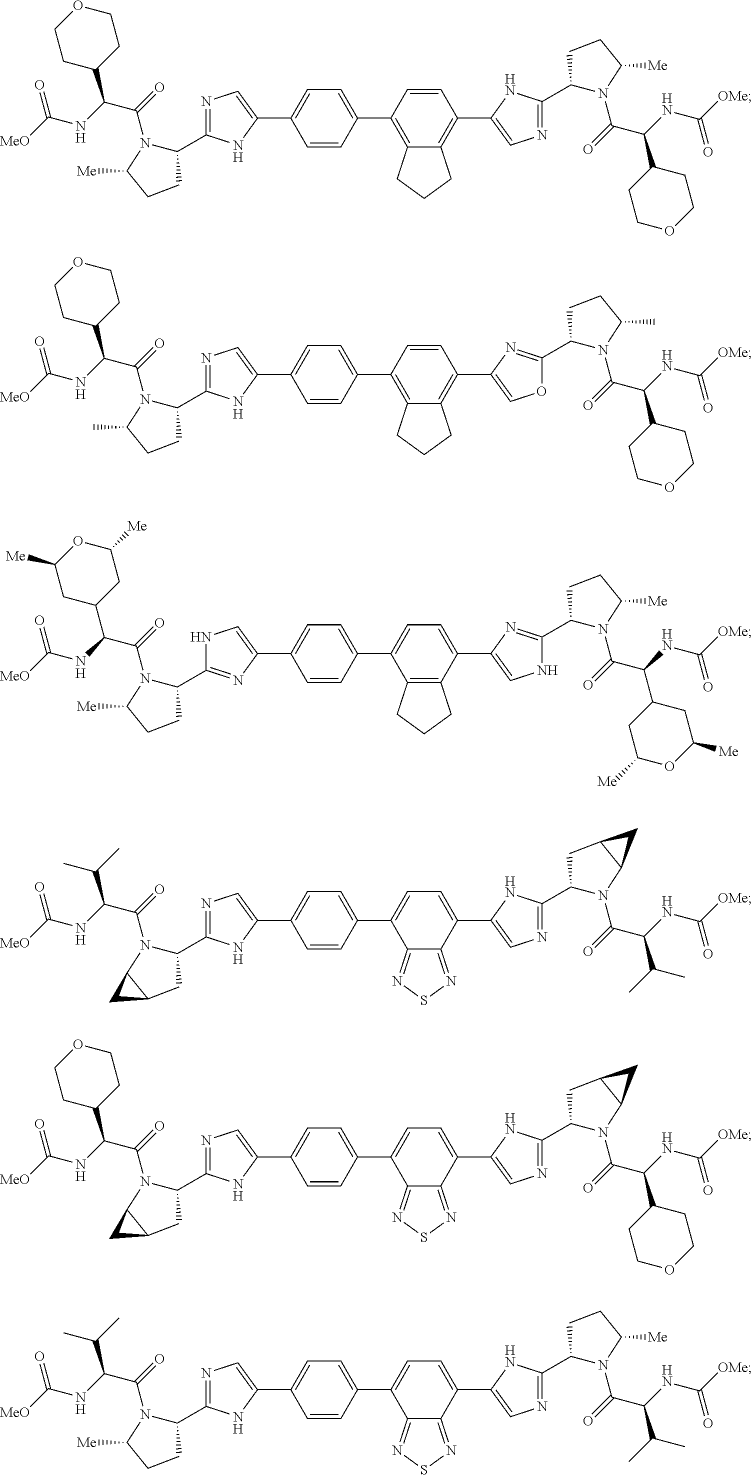 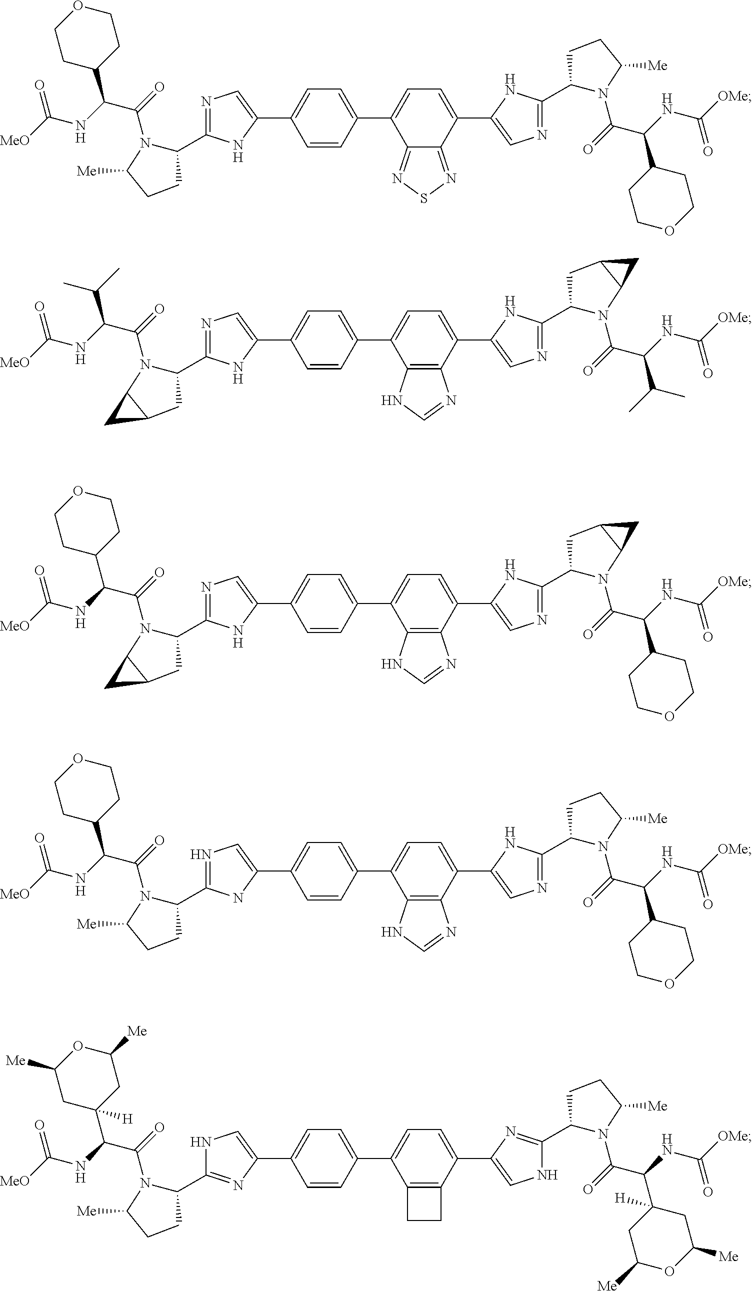 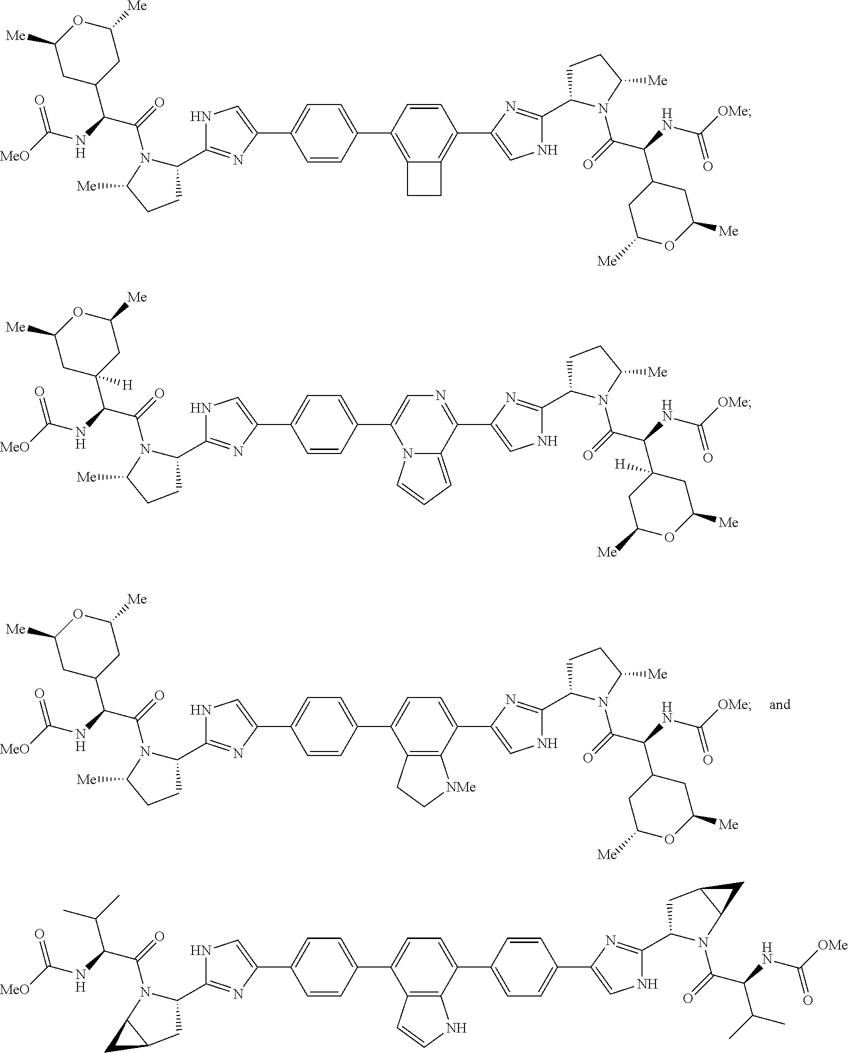
or a pharmaceutically acceptable salt thereof.

8. A composition comprising a compound of claim 1, or a pharmaceutically acceptable salt thereof, and a pharmaceutically acceptable carrier.

9. The composition of claim 8 further comprising one or two additional compounds having anti-HCV activity.

10. The composition of claim 9 wherein at least one of the additional compounds is an interferon or a ribavirin.

11. The composition of claim 10 wherein the interferon is selected from interferon alpha 2B, pegylated interferon alpha, pegylated interferon lambda, consensus interferon, interferon alpha 2A, and lymphoblastiod interferon tau.

12. The composition of claim 9 wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.

13. A method of treating an HCV infection in a patient, comprising administering to the patient a therapeutically effective amount of a compound of claim 1, or a pharmaceutically acceptable salt thereof.

14. The method of claim 13 further comprising administering one or two additional compounds having anti-HCV activity prior to, after or simultaneously with the compound of claim 1, or a pharmaceutically acceptable salt thereof.

15. The method of claim 14 wherein at least one of the additional compounds is an interferon or a ribavirin.

16. The method of claim 13 wherein interferon is selected from interferon alpha 2B, pegylated interferon alpha, pegylated interferon lambda, consensus interferon, interferon alpha 2A, and lymphoblastiod interferon tau.

17. The method of claim 13 wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.
